(19) United States
(12) Patent Application Publication
(10) Pub. No.: US 2025/0275988 A1
(43) Pub. Date: Sep. 4, 2025

(54) FORMULATION FOR TREATING URINARY SYSTEM DISORDERS

(71) Applicant: WATERSHED MEDICAL, INC., Bellevue, WA (US)

(72) Inventors: Christopher R. KINSELLA, JR., Bellevue, WA (US); Saniya ALI, Bellevue, WA (US); Clay Justin NOLAN, Bellevue, WA (US); Rush Lloyd BARTLETT, II, Bellevue, WA (US)

(73) Assignee: WATERSHED MEDICAL, INC., Bellevue, WA (US)

(21) Appl. No.: 18/858,722

(22) PCT Filed: Apr. 19, 2023
(86) PCT No.: PCT/US2023/065965

§ 371 Date: Oct. 21, 2024

Related U.S. Application Data
(60) Provisional application No. 63/332,639, filed on Apr. 19, 2022.

(51) Int. Cl. A61K 31/635; A61K 9/00; A61K 9/16; A61K 31/155; A61K 31/407; A61K 31/427; A61K 31/43; A61K 31/431; A61K 31/496; A61K 31/546; A61K 31/551; A61K 31/65; A61K 31/7036; A61P 13/02; A61P 13/10

(52) U.S. Cl. CPC A61K 31/635; A61K 9/0034; A61K 9/1617; A61K 9/1641; A61K 9/1652; A61K 9/167; A61K 31/155; A61K 31/407; A61K 31/427; A61K 31/43; A61K 31/431; A61K 31/496; A61K 31/546; A61K 31/551; A61K 31/65; A61K 31/7036; A61P 13/02; A61P 13/10

(57) ABSTRACT

Embodiments include intravesical therapies for the treatment of ailments such as urinary system disorders. A controlled release drug delivery system is administered into the urinary bladder of a patient. Controlled release particles in the formulation can include a pharmacologically active agent and an excipient (i.e., controlled release carrier). Particle buoyancy in urine and size ensure that the formulation is retained within a patient's bladder and releases the active agent into the bladder through the duration of an extended drug delivery time period. Methods of treating urinary system disorders by intravesicular administration of the particle formulation are also described, and include methods of treating urinary system infections, bladder cancer, incontinence, and other urinary system disorders.

### RELATED APPLICATIONS

This application claims priority to U.S. provisional patent application Ser. No. 63/332,639 filed on Apr. 19, 2022. The contents of the aforementioned application are incorporated herein by reference.

### FIELD OF THE INVENTION

The invention relates generally to the treatment of urinary system disorders, and, more particularly, relates to a formulation and method for the intravesicular administration of a pharmacologically active agent to treat or prevent a disorder of the urinary system.

### BACKGROUND

A urinary tract infection (“UTI”) is an infection that affects part of the urinary tract. When it affects the lower urinary tract it is known as a bladder infection (i.e., cystitis) and when it affects the upper urinary tract it is known as a kidney infection (i.e., pyelonephritis). Symptoms of a urinary tract infection include pain with urination, frequent urination, and feeling the need to urinate despite having an empty bladder. The most common cause of infection is Escherichia coli, though other bacteria or fungi may sometimes be the cause. Risk factors include female anatomy, sexual intercourse, diabetes, obesity and family history. In uncomplicated cases, UTIs are treated with a short course of antibiotics (e.g., nitrofurantoin).

The prevalence of UTI risk increases with age. They are particularly common in older adults who use catheters or live in a nursing home or other full-time care facility. Other conditions common in older adults (e.g., Alzheimer's disease, Parkinson's disease and diabetes) can lead to urinary retention or neurogenic bladder which increases the risk of UTIs. According to a recent study, more than one-third of all infections in people in nursing homes are UTIs. More than 10 percent of women over age 65 report having a UTI within the past year. That number increases to almost 30 percent in women over 85. Men also tend to experience more UTIs as they age. In many cases, chronic urinary tract infections require continuous medication, which leads to progressive levels of resistance to antibiotics and ultimately to kidney problems.

If not treated timely or properly, an infection from a UTI can spread. A UTI can ultimately lead to a bladder infection. This presents the risk of the infection spreading to the kidney via the ureter which can lead to more serious consequences including kidney failure and sepsis (i.e., urosepsis). This occurs frequently in elderly patients. In many cases, elderly patients do not present typical signs of infection until they become septic at which point hospitalization is needed.

UTIs and complications caused by UTIs are also expensive and as they often require hospitalization. In many cases, a UTI is only treated after the infection has developed and becomes symptomatic. Current treatments aimed at preventing the formation of UTIs require continuous prophylactic oral medication that have side-effects common with long-term medication. Further, long term consumption of antibiotics can lead to drug resistant bacteria. In many cases, UTIs reduce the quality of life, in particular when kidney damage leads to kidney failure requiring dialysis or implantation of a donor kidney.

For conditions related to urinary tract infection, clearance of the bacteria is improved with complete emptying of the bladder. If the bladder does not empty efficiently, there is a retained volume of urine termed the post-void residual (PVR), which can act as a reservoir of bacteria for subsequent and ongoing infection. Detecting and minimizing this PVR faces several challenges. PVR can be measured indirectly through ultrasound assessment. However, this method has limited accuracy depending on body habitus and at volumes less than 50 ml and greater than 250 ml. Alternatively, PVR can be measured and removed by draining the remaining urine through a catheter inserted retrograde through the urethra. However, this method can be painful and psychologically traumatizing.

PVR can be decreased by removing the primary cause of bladder outlet obstruction, such as widening an area of urethral narrowing. However, this approach often requires a separate procedure or surgery. Removal of the PVR without treating the underlying cause for the retained urine is a temporary maneuver as the volume will reaccumulate by the next voiding cycle. Without a procedure or surgery, this volume will remain and can be a potential reservoir of future and ongoing infection.

Conventionally, UTIs are treated with oral antibiotics. A high dose of antibiotics is needed so that an effective amount reaches the urinary tract. With systemic delivery of drugs to treat UTIs, the volume of distribution of the therapy is equal to the total amount of urine that enters the bladder. Therefore, the therapy is voided with each emptying of the bladder and requires constant replenishment. In other cases, the drug target can be limited (i.e., the bladder wall) but cannot practically be reached without raising the concentration of the drug in the urine to the desired therapeutic concentration.

There is a need for improved methods of preventing and treating UTIs. Such methods should target bacteria within the bladder/urinary tract without side-effects or risks associated with oral administration of oral antibiotics. The compositions and methods described herein satisfy these objectives and can especially benefit patients at increased risk of a UTI such as the elderly or incapacitated. The system and methods described herein can also be used to treat other ailments such as, for example, overactive bladder and other urinary system disorders.

### SUMMARY OF THE INVENTION

The inventions described and claimed herein have many attributes and embodiments including, but not limited to, those set forth or described or referenced in this brief summary. The inventions described and claimed herein are not limited to, or by, the features or embodiments identified in this summary, which is included for purposes of illustration only and not restriction.

Embodiments include a system of intravesicular administration (i.e., drug delivery locally in the bladder) with tunable and controlled drug release via a non-permanent carrier. In aspects, the product (i.e., carrier or controlled release carrier) stays afloat in urine to allow for retention in bladder. In aspects, an active agent (i.e., drug) remains in bladder to provide therapeutic drug target concentration for therapy duration.

Embodiments include a system and method of treating an ailment such as a urinary system disorder. In aspects, the system and method include a pharmaceutical formulation for intravesicular administration of a pharmacologically active agent. The formulation can include particles, each particle having an excipient portion and an active agent. In aspects, the excipient portion is a degradable material.

The agent can be, for example, an antibiotic, an anti-cancer agent. The ailment can be, for example, an infection (e.g., UTI), over-active bladder, interstitial cystitis, anticoagulant disease, underactive bladder, retained urine, diabetes, heart failure, kidney failure or cancer (e.g., bladder cancer, kidney cancer, ureter cancer or urethra cancer).

In aspects, the agent in an anti-infective agent (i.e., an antibacterial, antiviral, antifungal or antiparasitic medication). In aspects, the active agent is an antimicrobial agent such as, for example, silver sulfadiazine, amikacin, tobramycin, cefoperazone-sulbactam, cefoperazone-avibactam, cefoperazone-tazobactam, piperacillin-tazobactam, chlorhexidine, doripenem, ertapenem, imipenem, meropenem, cefepime, cefoxitin, ceftazidime, tigecycline, colistin, polymyxin B or aztreonam.

In aspects, the particles are cylindrically-shaped or spherically-shaped. In aspects, the particles have a hollow core. In aspects, the particles have a mean diameter of about 2 to 8 mm. Alternatively, the particles have a mean diameter of about 2 to 4.5 mm or about 2 to 6 mm.

In aspects, the particles are comprised of fatty acids and/or wax. In aspects, a mucoadhesive material is included in the particles.

In aspects, the particles are comprised of a matrix and the pharmacologically active agent is dispersed therein.

In aspects, the controlled release carrier gradually dissolves, degrades, or erodes in urine to release the active agent from the microparticles in the bladder. In aspects, the particles are modified to adjust the drug delivery time period.

In aspects, a primary population of particles and a secondary population are implanted, each with different qualities (e.g., different active agent).

In aspects, the particles include a color indicator (e.g., methylene blue) that is excreted in the urine and allows a patient to visualize one or more phases of a treatment.

In aspects, the system includes a first subset of particles and a second subset of particles, each with different qualities (e.g., size, shape, composition, active agent, etc.).

In aspects, the product is cleared from bladder over time, either by behavioral change (loss of buoyancy) and urinated from the bladder or mechanical change (morselization, surface erosion) or chemical change (biodegradable) within, for example, two to six months after implantation. In aspects, the product is delivered to a patient via the urethra with a syringe with a diameter less than six millimeters. In aspects, the product and its materials are biocompatible.

In embodiments, the density of the carrier particles is less than 1.030 specific gravity of urine.

Embodiments also include a method of extending the duration of retention of a pharmaceutical agent in the bladder. In aspects, the duration of retention can be tuned/adjusted by, for example, changing the shape, size, excipient composition, and/or method of production of the particles.

In embodiments, an active agent is released locally in the bladder in urine at a zero-order, steady state at or above therapeutic levels for a duration of time (e.g., up to three months from implantation). In aspects the active agent is released for a duration that is suited for a particular ailment to be treated (e.g., about one week, about two weeks, about three weeks, about one month, about six weeks, about two months, about three months, about four months, about five months, about six months or longer).

Embodiments also include a method of treating an infection (e.g., UTI). In aspects, the method includes intravesicular administration of an effective amount of a composition that includes controlled release particles described herein conjugated with an antibiotic (e.g., amikacin).

Embodiments include a method of treating bladder cancer (e.g., urothelial carcinoma). In aspects, the method includes intravesicular administration of an effective amount of a composition that includes controlled release particles described herein conjugated with an anti-neoplastic agent (e.g., taxane).

Additional embodiments include methods of treating or preventing UTI by administering the formulations described herein. In aspects, the methods are used with patients who are at risk of UTI and/or have a history of UTIs.

Accordingly, embodiments include a method of treating and/or preventing an ailment. The method can include steps of (a) providing a plurality of particles, each particle having a buoyancy resulting in flotation of the plurality of particles in urine contained in a urinary bladder. (b) delivering the plurality of particles into the urinary bladder of the subject and (c) allowing excretion and/or degradation of the plurality of particles over a period of time. Each particle can include an excipient portion and an active agent, and wherein the active agent is released into the urinary bladder.

In aspects, the ailment is a urinary tract infection (UTI), bladder cancer, kidney cancer, ureter cancer, urethra cancer, anticoagulant disease, overactive bladder, underactive bladder, retained urine, diabetes, heart failure, kidney failure or cystitis.

In aspects, the active agent is released into the urine at a substantially steady state. In aspects, the step of delivering the plurality of particles into the urinary bladder disrupts biofilm with the urethra or bladder.

In aspects, the method includes a step of diagnostic imaging.

### Definitions

Reference in this specification to “one embodiment/aspect” or “an embodiment/aspect” means that a particular feature, structure, or characteristic described in connection with the embodiment/aspect is included in at least one embodiment/aspect of the disclosure. The use of the phrase “in one embodiment/aspect” or “in another embodiment/aspect” in various places in the specification are not necessarily all referring to the same embodiment/aspect, nor are separate or alternative embodiments/aspects mutually exclusive of other embodiments/aspects. Moreover, various features are described which may be exhibited by some embodiments/aspects and not by others. Similarly, various requirements are described which may be requirements for some embodiments/aspects but not other embodiments/aspects. Embodiment and aspect can be in certain instances be used interchangeably.

The terms used in this specification generally have their ordinary meanings in the art, within the context of the disclosure, and in the specific context where each term is used. Certain terms that are used to describe the disclosure are discussed below, or elsewhere in the specification, to provide additional guidance to the practitioner regarding the description of the disclosure. It will be appreciated that the same thing can be said in more than one way.

Consequently, alternative language and synonyms may be used for any one or more of the terms discussed herein. Nor is any special significance to be placed upon whether or not a term is elaborated or discussed herein. Synonyms for certain terms are provided. A recital of one or more synonyms does not exclude the use of other synonyms. The use of examples anywhere in this specification including examples of any terms discussed herein is illustrative only, and is not intended to further limit the scope and meaning of the disclosure or of any exemplified term. Likewise, the disclosure is not limited to various embodiments given in this specification.

Without intent to further limit the scope of the disclosure, examples of instruments, apparatus, methods and their related results according to the embodiments of the present disclosure are given below. Note that titles or subtitles may be used in the examples for convenience of a reader, which in no way should limit the scope of the disclosure. Unless otherwise defined, all technical and scientific terms used herein have the same meaning as commonly understood by one of ordinary skill in the art to which this disclosure pertains. In the case of conflict, the present document, including definitions, will control.

The term “over-active bladder” or “OAB” refers to a group of urinary symptoms (rather than a disease). The most common symptom is a sudden, uncontrolled need or urge to urinate. Some people will leak urine when they feel this urge. Another symptom is the need to pass urine many times during the day and night. OAB can be described as a constant urge to urgently urinate. Leaking urine is called “incontinence.”

The term “stress urinary incontinence” or “SUI” refers to another common bladder problem. Subjects with SUI leak urine while sneezing, laughing or doing other physical activities.

The term “interstitial cystitis,” “IC” or “bladder pain syndrome” refers to a chronic, or long-lasting, condition that causes painful urinary symptoms. Symptoms of IC can be different from person to person. For example, some people feel mild discomfort, pressure, or tenderness in the pelvic area. Others may have intense pain in the bladder or struggle with urinary urgency, the sudden need to urinate, or frequency, the need to urinate more often. Health care professionals diagnose IC by ruling out other conditions with similar symptoms.

The term “active agent” or “active ingredient” refers to a substance, compound, or molecule, which is biologically active or otherwise, induces a biological or physiological effect on a subject to which it is administered to. In other words, “active agent” or “active ingredient” refers to a component or components of a composition to which the whole or part of the effect of the composition is attributed. An active agent can be a primary active agent, or in other words, the component(s) of a composition to which the whole or part of the effect of the composition is attributed. An active agent can be a secondary agent, or in other words, the component(s) of a composition to which an additional part and/or other effect of the composition is attributed.

The term “pharmaceutically acceptable” is meant a material that is not biologically or otherwise undesirable, i.e., the material may be incorporated into a pharmaceutical composition as provided herein and not cause any substantial undesirable biological effects or interact in a deleterious manner with any of the other components of the composition. When the term “pharmaceutically acceptable” is used to refer to a solid or semi-solid carrier, liquid vehicle, or other excipient, i.e., to any inactive ingredient herein, it is implied that the carrier or excipient has met the required standards of toxicological and manufacturing testing and/or that it is included on the Inactive Ingredient Guide prepared by the U.S. Food and Drug Administration and designated “Generally Regarded as Safe” (“GRAS”).

The term “controlled release” refers to a mechanism of drug delivery wherein administration of an active agent-containing formulation or fraction thereof does not result in the immediate release of 100% of the active agent. This allows medication levels to remain at an effective level for a duration of time. The term can be used interchangeably with “nonimmediate release” as defined in Remington: The Science and Practice of Pharmacy, Nineteenth Ed. (Easton, PA: Mack Publishing Company, 1995). In general, the term “controlled release” as used herein includes sustained release, modified release, pulsatile release, and delayed release formulations, as well as formulations that combine two or more types of release profiles, such as immediate release of a bolus dose followed by pulsatile release or sustained release thereafter. “Sustained release” (synonymous with “extended release”) refers to a formulation that provides for gradual release of an active agent over an extended period of time, and that preferably, although not necessarily, results in substantially constant levels of an agent in a volume of distribution (e.g., urine, bladder, total body water, etc.), and is normally referred to as “zero order” release. “Controlled release” also includes “delayed release,” indicating a formulation that, following administration to a patient, provides for a measurable time delay before the active agent is released from the formulation into the patient's body. Controlled release dosage forms herein, however, are generally of the sustained release type.

The term “particles” or “microparticles” refers to a carrier or controlled release carrier described herein that is formulated so as to contain a pharmacologically active agent. The particles can be substantially spherical, cylindrical or rod-like or have some other shape. Particle size is given as the mean particle diameter in a population of particles. The particle size distribution in a population of particles is relatively narrow (i.e., the specified mean particle size is associated with a fairly low standard of deviation, typically less than 30%), for example, less than 20% or less than 15%. Particles within a formulation may or may not have substantially the same shape and size.

The term “intravesicular administration” or “intravesicular therapy” refers to a treatment wherein a therapeutic is put directly into the bladder (i.e., through a catheter) rather than being given orally or injected into a vein. Similarly, an intravesical drug is directly administered into the urinary bladder via a urethral catheter.

The term “mucoadhesion” refers to the adhesion between two materials, one of which is a mucosal surface. Mucoadhesive drug delivery systems prolong the residence time of the dosage form at the site of application or absorption. Mucoadhesive drug delivery systems interact with the mucus layer covering the mucosal epithelial surface, and mucin molecules and increase the residence time of the dosage form at the site of absorption.

The term “spherical” herein refers to a precisely spherical particle or, more generally, to a particle with a rounded surface may or may not be substantially spherical.

The terms “mean diameter,” “mean longest dimension,” and “particle size” are used interchangeably herein. As used herein, the particle size is given by mean diameter for spherical particles and mean longest dimension for non-spherical particles. It should be noted that the term “mean diameter” technically corresponds to substantially spherical particles; however, other particle shapes can also be incorporated into the particulate formulation, providing that the particles have at least one dimension (i.e., at least one mean dimension, in the overall population of particles) greater than 2.0 mm, e.g., in the range of 2.0 mm to about 12.0 mm, e.g., about 2.5 mm to about 6.5 mm, e.g., about 2.5 mm to about 6.0 mm, about 2.5 mm to about 5.5 mm, about 2.5 mm to 5.0 mm, etc., as before. An elongated particle, e.g., a cylindrical or rod-like particle, could, for example, have a length of about 9.0 and yet be narrow enough to pass through a urethral catheter and the urethral opening.

The terms “effective amount” and “therapeutically effective amount” of an active agent, an active agent combination, or a pharmaceutical formulation refer to an amount or concentration that is nontoxic but sufficient for producing a desired result. The exact amount required will vary from subject to subject, depending on factors such as the age, weight and general condition of the subject, the particular condition being treated, the severity of the condition, the specific active agent, and the judgment of the clinician.

The term “urinary system” is used in the conventional sense to refer to the lower urinary tract as well as the upper urinary tract, and thus includes the bladder, urethra, kidneys, and ureters.

The term “disorder” refers to a physiological condition of clinical relevance and thus includes symptomatic or asymptomatic conditions regardless of etiology. Disorders thus include adverse conditions resulting from disease or injury. The disorders addressed with the present invention are disorders of the urinary system.

A “drug delivery time period” refers to a period of time during which a pharmacologically active agent is released from a formulation or fraction thereof, generally an extended time period.

The term “substantially” indicates the possibility of slight deviation from a recited chemical or physical property and allows for a difference of at most about 20%, or at most about 10%, or at most about 5%, between an actual chemical or physical property and the recited chemical or physical property. The term “substantially homogeneous,” for example, refers to a material in the form of a mixture of two or more components in which the material is substantially uniform throughout, with any two discrete regions within the material differing by at most about 20%, or at most about 10%, or at most about 5%, with respect to a chemical or physical property of the material, such as the presence or absence of a component, the concentration of a component, the degree of hydrophilicity or lipophilicity, density, crystallinity, or the like. Similarly, the term “approximately” or “about” in any context is intended to connote a possible variation of at most about 20%. Generally, the term connotes a possible variation of at most about 10%, or at most about 5%.

The term “subject” or “patient” refers to any single animal, more preferably a mammal (including such non-human animals as, for example, dogs, cats, horses, rabbits, zoo animals, cows, pigs, sheep, and non-human primates) for which treatment is desired. Most preferably, the patient herein is a human.

The term “pharmaceutically acceptable carrier” as used herein refers to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutically acceptable composition” as used herein refers to a composition comprising at least one compound as disclosed herein formulated together with one or more pharmaceutically acceptable carriers.

The term “treating” or “treatment” refers to one or more of (1) inhibiting the disease; e.g., inhibiting a disease, condition or disorder in an individual who is experiencing or displaying the pathology or symptomatology of the disease, condition or disorder (i.e. arresting further development of the pathology and/or symptomatology); and (2) ameliorating the disease; e.g., ameliorating a disease, condition or disorder in an individual who is experiencing or displaying the pathology or symptomatology of the disease, condition or disorder (i.e. reversing the pathology and/or symptomatology) such as decreasing the severity of disease.

The term “specific gravity” refers to the ratio of a material's density with that of water at 4° C. It is therefore a relative quantity with no units. The specific gravity of urine refers to the electrolytes and urine osmolality. The normal range for urine specific gravity is 1.005 to 1.030. Specific gravity, in the context of clinical pathology, is a urinalysis parameter commonly used in the evaluation of kidney function and can aid in the diagnosis of various renal diseases.

The term “effervescence” refers to the escape of gas from an aqueous solution and the foaming or fizzing that results from that release. In aspects, particles described herein include an effervescent substance. For example, an effervescent reaction between carbonate/bicarbonate salts and citric/tartaric acid liberates carbon dioxide, which gets entrapped in the jellified hydrocolloid layer of the system, thus decreasing its specific gravity and making it float over time. Resin beads can be loaded with bicarbonate and coated with ethyl cellulose. The coating (which is insoluble but permeable) allows permeation of water. Thus, carbon-dioxide is released, causing the beads to float in urine. Another approach is to incorporate sodium bicarbonate directly into the polymer and drug melt. As urine/water comes in contact with sodium bicarbonate, the formulation floats.

The term “biocompatibility” refers to the ability of a material to perform with an appropriate host response in a specific situation. A material that is biocompatible will not have toxic nor injurious effects on biological systems. For example, an implanted device will exist in harmony with tissue without causing deleterious changes.

The term “WITEPSOL®” refers to an NF grade, small pastille form hard fat suppository base comprised of glycerides from vegetable origins with a history of use in a range of APIs.

The term “amikacin” refers to an antibiotic medication used for a number of bacterial infections. This includes joint infections, intra-abdominal infections, meningitis, pneumonia, sepsis, and urinary tract infections (UTIs). It is also used for the treatment of multidrug-resistant tuberculosis. It is typically used by injection into a vein using an IV or into a muscle.

The term “excipient” refers to a pharmacologically inert ingredients added intentionally to a drug product for various functional roles, such as to enhance dosage form volume or size, disintegration of solid dosage forms, binding of particulates, lubrication during processing, taste masking or modifying drug release.

The term “zero-order” refers to a delivery system that releases a drug at a constant rate (i.e., the release rate is independent of the concentration or the amount of drug that remains in the delivery system). Drug release kinetics is said to be zero-order kinetics when a constant amount of drug is eliminated per unit time but the rate is independent of the concentration of the drug. Zero-order drug delivery systems (DDS) can overcome the issues faced by immediate-release and first-order systems by releasing the drug at a constant rate, thereby maintaining drug concentrations within the therapeutic window for an extended period.

The term “first-order” refers to a drug release rate that is directly proportional to the concentration gradient and is a function of the amount of drug remaining in the dosage form. Similarly, “sustained release” refers to the slow release of a drug over an extended period after administration of a single dose.

The terms “therapeutic index,” “TI” or “therapeutic ratio” refer to a quantitative measurement of the relative safety of a drug. It is a comparison of the amount of a therapeutic agent that causes the therapeutic effect to the amount that causes toxicity. A therapeutic window or safety window refers to the range of doses that optimize between efficacy and toxicity, achieving the greatest therapeutic benefit without resulting in unacceptable side effects or toxicity. TI is calculated from the ratio of the dose of a drug that causes adverse effects at an incidence/severity not compatible with the targeted indication (e.g., toxic dose in 50% of subjects, TD₅₀) to the dose that leads to the desired pharmacological effect (e.g., efficacious dose in 50% of subjects, ED₅₀).

Other technical terms used herein have their ordinary meaning in the art that they are used, as exemplified by a variety of technical dictionaries. The particular values and configurations discussed in these non-limiting examples can be varied and are cited merely to illustrate at least one embodiment and are not intended to limit the scope thereof.

### BRIEF DESCRIPTION OF THE DRAWINGS

FIG. 1 schematically illustrates the method for making particles as described in Example 4.

FIG. 2 sets forth the results of the evaluation of antimicrobial efficacy of silver sulfadiazine (SSD) and cefpodoxime as described in Example 2.

FIG. 3 is a graph illustrating the percentage of SSD released over time from particles of the invention as determined in Example 5. As discussed in the example, the results indicate that a zero-order release profile was achieved.

FIG. 4 is a graph illustrating the cumulative amount of SSD released over time as a percentage of the amount of drug originally present, for glyceryl tristearate (GTS)/SSD particle formulations prepared in phosphate buffered saline (PBS) at pH 7.5, as explained in Example 6.

FIG. 5 is a graph illustrating the cumulative amount of SSD released over time as a percentage of the amount of drug originally present, for glyceryl tristearate (GTS)/SSD particle formulations prepared in artificial urine media at pH 7.5, as explained in Example 6.

FIG. 6 illustrates the results obtained for the degradation assay of Example 7, in which carrier degradation over time was evaluated for GTS/SSD particles.

FIG. 7 provides scanning electron microscope (SEM) images of the particles evaluated in Example 7 and shows that the microsphere surfaces appear unchanged after seven days.

FIG. 8 provides a list of the carriers and vehicles evaluated as described in Example 8.

FIG. 9 provides the results of mucoadhesive testing carried out as described in Example 8.

FIG. 10 illustrates the mucoadhesion and degradation rates observed with particles formulated with different controlled release carriers.

FIG. 11 illustrates mucoadhesive ability of particles formulated with several different controlled release carriers identified in FIG. 10.

FIG. 12 illustrates the effect of carrier loading on drug release rate, as described in Example 8, for particles prepared with SSD and stearyl alcohol.

FIG. 13 provides the results of buoyancy studies carried out with various controlled release carriers and combinations, also as described in Example 8.

FIG. 14 models the rate of formulation retention over time, as also described in Example 8.

FIG. 15A depicts a cylindrically-shaped hollow rod.

FIG. 15B shows the buoyancy of rods after 14 days (cylindrical and solid).

FIG. 15C shows the effect of carrier loading for particles prepared as solid rods and rod with a hollow core (μg/ml of SSD vs. time).

FIG. 15D shows the effect of carrier loading for particles prepared as solid rods and rods with a hollow core (percent release vs. time).

FIG. 16A shows the chemical structure of amikacin.

FIG. 16B the chemical structures of amikacin and pamoic acid.

FIG. 16C illustrates the rate of amikacin release over time (μg/ml vs. time).

FIG. 17A shows the shapes, volume and surface area of the particles tested.

FIG. 17B illustrates the effect different shapes (cylinders and rods of different sizes) on drug release (percent release vs. time).

FIG. 17C illustrates the effect different shapes (cylinders and rods of different sizes) on drug release (concentration vs. time).

FIG. 18A illustrates the effect different excipients on drug release using spheres (percent release vs. time).

FIG. 18B illustrates the effect different excipients on drug release using spheres (concentration vs. time).

FIG. 19A illustrates the effect different excipients on drug release using cylinders (percent release vs. time).

FIG. 19B illustrates the effect different excipients on drug release using cylinders (concentration vs. time).

FIG. 20A illustrates the effect different excipients on drug release using spheres (percent release vs. time).

FIG. 20B illustrates the effect different excipients on drug release using spheres (percent release vs. time).

FIG. 21A illustrates the use of varying amounts of PAA in formulations on drug release (μg/ml vs. time).

FIG. 21B illustrates the use of varying amounts of PAA in formulations on drug release (percent release vs. time).

FIG. 22A illustrates the difference in drug release over time for stirred and homogenized particles (μg/ml vs. time).

FIG. 22B illustrates the difference in drug release over time for stirred and homogenized particles (percent release vs. time).

FIG. 22C is a comparison of the results of particles prepared by (a) stirring and (b) homogenizing.

FIG. 23 illustrates the profile of the release of amikacin from particles (μg/ml vs. time).

FIG. 24 illustrates the effect of formulation process variables.

FIG. 25 illustrates the amikacin rapid releasing formulation.

### DETAILED DESCRIPTION

It is to be understood that both the foregoing general description and the following detailed description are exemplary and explanatory and are intended to provide further explanation of the subject technology as claimed. Additional features and advantages of the subject technology are set forth in the description below, and in part will be apparent from the description, or may be learned by practice of the subject technology. The advantages of the subject technology will be realized and attained by the structure particularly pointed out in the written description and claims hereof.

Embodiments of the invention include a platform and methods for treating ailments related to the urinary system. Intravesiclular particles deliver medication to specifically increase the drug concentration in the bladder/urethra. This avoids the need for oral medication and systemic administration. With intravesicular administration, lower doses of antibiotic are effective and consequently there are fewer side effects. An improved therapeutic window is also possible with controlled drug release.

Accordingly, the invention provides a formulation and method for the controlled release of a pharmacologically active agent within the bladder to treat a disorder of the urinary system. A formulation that includes active agent-containing particles is administered to the subject via the intravesicular route (i.e., through the urethra or via a bladder injection). Because the particles are buoyant in urine, they float to the surface of urine that is present in the bladder. The particles can remain in the bladder for the duration of an extended drug delivery time period. As they gradually degrade, the particles gradually dissolve/break apart inside the bladder and are excreted.

In aspects, the type of particles used (e.g., size, shape, composition and method produced) can be chosen based on a proposed treatment. For example, a two millimeter cylindrically-shaped particle (with an antibiotic active agent) may be optimal for treating a female patient with a UTI. These relatively large particles (wherein 2 mm is the mean longest dimension) can be retained within the bladder in three ways. First, the majority of the particles in the formulation (i.e., more than about 90%) are buoyant in urine. This buoyancy keeps the particles away from the urethral opening during the first half of the voiding cycle when the urethral opening is at its largest. Second, as the bladder empties and the urethral opening gradually narrows, the particle diameter is wider than the narrowed urethral opening in the second half of the voiding cycle and as voiding approaches completion. Third, at the end of the voiding cycle (when the urethral opening is narrowed and the buoyant particles are brought to the level of the urethral opening) the particles tend to aggregate within the bladder and thereby prevent outflow of the particles.

In aspects, the particles can include a mucoadhesive material. Upon voiding, the bladder contracts and the thick, irregular mucosal tissue (i.e., rugae) on the interior walls of the bladder can trap the microparticles in the resulting folds. The release of active agent within the bladder continues over a series of cycles in which the bladder refills and empties with urine. Furthermore, particle size, degradation or aggregation of microparticles, and other factors can be varied to facilitate retention within the bladder without necessarily requiring that microparticles be trapped in the rugae as the bladder contracts. Any of these retention means can be used in combination, and some may exhibit synergy with respect to enhancing the mechanistic process of an alternative retention means.

Another benefit involves penetration of biofilms. Contact between the particles and the interior walls of the bladder or urethra, as well as particle movement within the bladder can disrupt biofilms. The particles can cause turbulence, prevent biofilm formation and facilitate mechanical disruption of any biofilm already present, in turn reducing the likelihood of adherence of bacterial cells to a biofilm matrix. By disrupting biofilms in the bladder and urethra, the formulation and method can further minimize or eliminate bacteria growth within the bladder and urethra.

Generally, the pharmacologically active agent is a drug for the treatment of a disorder of the urinary system (i.e., a disorder of the bladder, ureters, urethra, kidney, or a combination). The selection of the pharmacologically active agent incorporated into the formulation is dependent on the particular disorder being treated. Treatment of urinary tract infections, particularly complicated urinary tract infections, requires administration of an anti-infective agent, which, is an anti-bacterial agent when the urinary tract infection is a bacterial infection. According to some embodiments, the anti-bacterial agent is elemental silver, silver ions, a silver salt, or a silver coordination compound.

The formulation is also useful in the treatment of urinary system disorders other than a bacterial infection, in which case the pharmacologically active agent is a drug other than an anti-bacterial agent. The active agent can be an anti-fungal or anti-viral agent; a chemotherapeutic agent; an anti-inflammatory agent; an anesthetic agent; an analgesic agent; a diuretic agent; a coagulant or anti-coagulant; a biologic; an agent for treatment of incontinence; a renin-angiotensin-aldosterone system (RAAS) inhibitor; an agent for treating kidney stones; or any other pharmacologically active agent effective to treat a urinary system disorder. In another aspect, the active agent is a contrast agent for diagnostics and monitoring.

The particles (i.e., controlled release carrier) can include multiple materials that render them buoyant within the bladder (i.e., buoyant in urine). Suitable carriers for rendering the particles buoyant in urine provide for a particle specific gravity that is less than about 1.03, more typically less than about 1.005. Generally, although not necessarily, and depending on the active agent and any excipients present, this means that the carrier itself has a specific gravity that is less than about 1.03, e.g., less than about 1.005.

The controlled release carrier includes a material that not only provides for buoyancy within the bladder but can also facilitate retention of the particles by the bladder wall (i.e., a mucoadhesive material). The particle material can be selected from waxes; fatty acid esters derived from a monohydric, dihydric, or polyhydric C₂-C₆ alcohol and a C₈-C₂₄ fatty acid; C₈-C₂₄ fatty acids; C₈-C₂₄ fatty alcohols; hydroxylated C₈-C₁₂ alkanes; polymers and copolymers of hydroxylated vinyl monomers; polymers and copolymers of carboxylated vinyl monomers; cellulosic polymers and derivatives thereof; and combinations of any of the foregoing. In embodiments wherein two or more mucoadhesive materials are used in combination, the combination can include a blend of two or more of the same type of mucoadhesive materials (e.g., two or more waxes, or two or more fatty acid esters) or a combination of two or more different types of mucoadhesive materials (e.g., a triglyceride and a fatty alcohol).

Accordingly, mucoadhesion, in addition to buoyancy and larger size particles (i.e., particles having a mean diameter of greater than 2.0 mm) can help ensure that the particles are retained within the bladder to provide for sustained release of the active agent throughout the extended drug delivery period.

According to further embodiments, the controlled release carrier includes a matrix and the pharmacologically active agent is dispersed therein. According to other embodiments, the particles are coated core-type controlled release dosage forms, wherein the controlled release carrier coats an active agent-containing core or wherein an active agent coating encloses a core of the controlled release carrier.

In any of these embodiments, the controlled release carrier, in addition to providing for buoyancy and retention, can be selected so that it gradually dissolves, degrades, or erodes in urine to gradually release the pharmacologically active agent from the particles into the bladder.

In another embodiment, the particles are designed to coalesce within the bladder. The microspheres may, in one example of such a formulation, be hemispherical, coalescing plane to plane to provide particle spheres within the bladder.

In another embodiment, the formulation includes a first subset of particles and a second subset of particles. The first subset of particles and the second subset of particles can differ in mean diameter (or in one or more other dimensions), shape, buoyancy, deformability, or in two or more of such properties. According to some embodiments, the first and second particle subsets differ in size. According to other embodiments, the first and second particle subsets differ in buoyancy. In still another embodiments, the first and second particle subsets differ with respect to both size and buoyancy. In each of the aforementioned examples, it is generally desirable for at least 90% of the particles of the formulation to be buoyant.

According to another embodiment, the first subset of particles, the second subset of particles, or both the first and second subsets of particles are reversibly deformable. In some embodiments, the first subset of particles, the second subset of particles, or both the first and second subsets of particles are deformable to an extent that allows flow within a chute having an inner diameter in the range of about 4 mm to about 6 mm, i.e., the diameter of the standard intravesicular catheter.

In any of the foregoing embodiments, the second subset of particles can include, relative to the first subset of particles, a different pharmacologically active agent, a different amount of the same or a different pharmacologically active agent, a different controlled release carrier, a different amount of controlled release carrier, or has a different controlled release profile.

The particles can be stored and administered in a solution (i.e., liquid vehicle). Accordingly, in another embodiment, the formulation additionally includes a liquid vehicle in which the population of particles is dispersed. The liquid vehicle typically includes one or more excipients (i.e., at least one of a viscosity adjusting agent, a tonicity adjusting agent, a buffer and a dispersant). As the formulation is intended for intravesicular administration, the liquid vehicle should be suitable for delivery via that route. As one of ordinary skill in the art will appreciate, low viscosity liquid vehicles are generally unsuitable for intravesicular administration; the liquid vehicle employed in the formulation should have a viscosity that is lower than that of castor oil or mineral oil, for example, within the range of normal body temperatures (about 36° C. to about 37° C.). In addition, hydrophilic vehicles or components should be avoided as such could compromise particle buoyancy in urine.

In another embodiment, the invention provides a controlled release pharmaceutical formulation for intravesicular administration to a subject to treat a bacterial infection of the bladder, the formulation can include:
(a) a population of particles having a mean diameter in the range of 2.5 mm to 4.0 mm and a mean specific gravity of less than 1.005, wherein the particles comprise about 50 wt. % to about 95 wt. % of a controlled release carrier that provides for sustained release of the active agent in the bladder throughout a drug delivery period of at least about one month, wherein the carrier is effective to render the particles buoyant in urine and comprises a mucoadhesive material to facilitate retention by the bladder wall; and
(b) about 5 wt. % to about 50 wt. % silver sulfadiazine dispersed within the controlled release carrier.

In another embodiment, a method is provided for treating a urinary system disorder in a subject, wherein the method comprises administering to the bladder a controlled release pharmaceutical formulation that includes a population of particles having a mean diameter (or at least one dimension) greater than 2.0 mm and comprised of about 2.5 wt. % to about 95 wt. % of a pharmacologically active agent effective to treat the urinary system disorder and 5 wt. % to 97.5 wt. % of a controlled release carrier effective to render the particles buoyant in urine and provide for sustained release of the active agent in the bladder throughout a drug delivery period of about one month to at least about three months. The urinary system disorder can be a disorder of the bladder, ureters, urethra, kidney, or a combination thereof.

In some embodiments, the urinary system disorder is an infection of the urinary tract (commonly referred to as a “UTI”) such as a bacterial, fungal, or viral infection. According to other embodiments, the method is used in the treatment of a cancer or benign tumor of the urinary system; urinary incontinence (including stress incontinence and overactive bladder, “OAB”) and urinary retention; urinary system inflammation, injury, or scarring; bladder pain syndrome; neurogenic bladder dysfunction; vesicoureteral reflux; a sexually transmitted disease such as chlamydia, gonorrhea, or syphilis; a bladder calculus; or a kidney stone, diabetic nephropathy, pyelonephritis, or kidney failure.

### Formulation Particles

In embodiments, the particles within the pharmaceutical formulation include (a) a pharmacologically active agent and (b) a controlled release carrier. By gradual reduction in mass and/or other means, the particles release the active agent into the bladder during an extended drug delivery time period. The components of the particles are described further below.

### A. Active Agents

As can be appreciated, the selection of pharmacologically active agent is dependent on the indication. Active agents that are administrable via intravesicular administration of a particle formulation of the invention are generally, although not necessarily, selected from the following categories: anti-infective agents, including anti-bacterial, anti-fungal, and anti-viral agents; chemotherapeutic agents; anti-inflammatory agents; anesthetic agents; analgesic agents; diuretic agents; coagulants and anti-coagulants; biologics; agents for treatment of incontinence, including overactive bladder (including antimuscarinic agents, β3-adrenergic receptor agonists, anesthetic agents, and analgesic agents); renin-angiotensin-aldosterone system (RAAS) inhibitors; agents for treating kidney stones; and contrast agents for diagnostics and monitoring. Specific examples of active agents that can be administered to treat a urinary system disorder as provided herein are as follows:

### Anti-Infective Agents:

Anti-infective agents include tetracycline antibiotics and related compounds (e.g., chlortetracycline, oxy-tetracycline, demeclocycline, methacycline, doxycycline, minocycline and roli-tetracycline);

Anti-infective agents also include macrolide antibiotics such as erythromycin, clarithromycin, and azithromycin; streptogramin antibiotics such as quinupristin and dalfopristin; beta-lactam antibiotics, including penicillins (e.g., penicillin G, penicillin VK), antistaphylococcal penicillins (e.g. cloxacillin, dicloxacillin, nafcillin and oxacillin), extended spectrum penicillins (e.g. aminopenicillins such as ampicillin, amoxicillin, and benzathine benzylpenicillin, and antipseudomonal penicillins such as carbenicillin), cephalosporins (e.g. cefadroxil, cefepime, cefpodoxime, cephalexin, cefazolin, cefoxitin, cefotetan, cefuroxime, cefotaxime, ceftazidime and ceftriaxone) and carbapenems such as imipenem, meropenem and aztreonam; aminoglycoside antibiotics such as streptomycin, gentamicin, tobramycin, amikacin and neomycin; glycopeptide antibiotics such as teicoplanin; sulfonamide antibiotics such as sulfacetamide, sulfabenzamide, sulfadiazine, sulfadoxine, sulfamerazine, sulfamethazine, sulfamethizole and sulfamethoxazole; silver-based active agents such as elemental silver, silver ions and salts, and silver coordination compounds; quinolone antibiotics such as levofloxacin, trovafloxacin, ciprofloxacin, nalidixic acid and ofloxacin; anti-mycobacterials such as isoniazid, rifampin, rifabutin, ethambutol, pyrazinamide, ethionamide, aminosalicylic and cycloserine; nitrofurans such as nitrofurazone and nitrofurantoin.

Anti-infective agents also include antifungal agents such as itraconazole, ketoconazole, fluconazole and amphotericin B; antiseptic agents such as the bisbiguanides chlorhexidine and alexidine; and miscellaneous antimicrobial agents such as trimethoprim, chloramphenicol, fosfomycin, spectinomycin, polymyxin B (colistin), bacitracin, nitrofurantoin, methenamine, and sodium oxychlorosene.

Agents for treating viral infections of the urinary system include, by way of example, anti-herpes agents such as aciclovir, famciclovir, foscarnet, ganciclovir, idoxuridine, sorivudine, trifluridine, valacyclovir and vidarabine; anti-retroviral agents such as didanosine, stavudine, zalcitabine, tenovovir and zidovudine; other antiviral agents including amantadine, interferon-α, ribavirin and rimantadine. Of particular interest for treatment of viral infections of the urinary system are cidofovir and leflunomide.

Antibacterial agents of particular interest herein are oligodynamic active agents (i.e., biocidal metal-based agents). Many such agents are silver-based, and, as noted above, include elemental silver, silver ions, silver salts, and silver coordination compounds. Silver salts can be inorganic, such as silver bromide, silver chloride, silver iodate, silver iodide, fosfomycin, silver oxide, silver perchlorate, and silver tetrafluoroborate. Organic silver-containing compounds include both organic silver salts and coordination compounds, for instance silver acetate, silver benzoate, silver carbonate, silver lactate, silver laurate, silver palmitate, and silver sulfadiazine (SSD). Other metals, such as gold, zinc, copper, and cerium, have also been found to possess antimicrobial properties, both alone and in combination with silver.

### SSD Degradation Products

Also of interest as antibiotic agents herein are degradation products of SSD that are formed in situ following intravesicular administration of SSD. These degradation products include, for example, sulfanilic acid; sulfanilamide; sulfaguanidine, the guanidine derivative of sulfanilamide; and 2-aminopyridine. It has now been discovered that these degradation products have anti-bacterial properties comparable to the original compound (i.e., prior to degradation in the bladder). The aforementioned SSD degradation products—which may result from enzymatic degradation, hydrolysis, physical erosion or degradation, or any mechanism—are therefore promising as active agents per se. This in turn means that when the SSD in a particle formulation of the invention is released into the bladder, the SSD and its degradation products can combine to provide an enhanced and prolonged therapeutic effect.

The selection of an anti-infective agent in any particular case will depend on the nature of the infective agent as well as other factors. Urinary system infections are commonly caused by the microorganisms Escherichia coli (E. coli), Klebsiella pneumonia, Staphylococcus saprophyticus, Proteus mirabilis, Enterococcus faecalis, Staphylococcus aureus, Candida albicans, Streptococcus agalactiae, Mycoplasma genitalium, Pseudomonas aeruginosa, Chlamydia trachomatis, and the herpes simplex viruses Human alphaherpesvirus 1 (HSV-1) and Human alphaherpesvirus 2 (HSV-2).

### Chemotherapeutic Agents:

Chemotherapeutic agents that can be administered via the intravesicular route using the particle formulations of the invention include, without limitation: apaziquone; aldesleukin; Bacillus Calmette-Guérin (BCG) immunotherapy; cisplatin; docetaxel; doxorubicin; erdafitinib; everolimus; fosfomycin; gemcitabine; methotrexate; mitomycin C; mitoxantrone; paclitaxel, thiotepa; camptothecin and its analogues and derivatives (e.g. 9-aminocamptothecin, 9-nitrocamptothecin, 10-hydroxycamptothecin, irinotecan, meglumine, topotecan and 20-O-β-glucopyranosyl camptothecin); taxanes (e.g. baccatins, cephalomannine and their derivatives); carboplatin; interleukin (IL)-2 and IL-12; interferon α-2a, interferon α-2b, interferon α-n3 and other agents of the interferon family; levamisole; altretamine; cladribine; tretinoin; procarbazine; dacarbazine; mitotane; asparaginase; porfimer; amifostine; mitotic inhibitors including podophyllotoxin derivatives teniposide and etoposide; and the vinca-alkaloids vinorelbine, vincristine and vinblastine. Of particular interest for the treatment of cancers of the urinary system, including bladder cancers, renal cancers, and cancers of the urethra and ureters include, without limitation, apaziquone, aldesleukin, axitinib, BCG immunotherapy, cisplatin, doxorubicin, erdafitinib, everolimus, fosfomycin, gemcitabine, IL-2, IL-12, methotrexate, mitomycin-C, thiotepa, vinblastine, and the monoclonal antibody medications bevacizumab (Avastin®), avelumab (Bavencio®), cabozantinib-S-malate, ipilimumab, nivolumab, sunitinib malate, and pembrolizumab, among others.

### Anti-Inflammatory Agents:

Active agents useful for treating inflammation of the urinary tract include nonsteroidal anti-inflammatory agents (NSAIDs) such as ketoprofen, flurbiprofen, ibuprofen, naproxen, fenoprofen, benoxaprofen, indoprofen, pirprofen, carprofen, oxaprozin, pranoprofen, suprofen, alminoprofen, butibufen, fenbufen, apazone, diclofenac, difenpiramide, diflunisal, etodolac, indomethacin, ketorolac, meclofenamate, nabumetone, phenylbutazone, piroxicam, sulindac and tolmetin; COX-2 inhibitors such as celecoxib, rofecoxib, and valdecoxib; and steroidal anti-inflammatory agents, e.g., hydrocortisone, hydrocortisone-21-monoesters (e.g. hydrocortisone-21-acetate, hydrocortisone-21-butyrate, hydrocortisone-21-propionate, hydrocortisone-21-valerate), hydrocortisone-17,21-diesters (e.g. hydrocortisone-17,21-diacetate, hydrocortisone-17-acetate-21-butyrate, hydrocortisone-17,21-dibutyrate), alclometasone, betamethasone, dexamethasone, flumethasone, prednisolone methylprednisolone, and triamcinolone.

Anti-inflammatory agents of particular interest in the treatment of urinary system inflammation are cromolyn sodium, pentosan polysulfate sodium, and the glycosaminoglycans chondroitin sulfate, hyaluronic acid, and heparin.

### Anesthetic Agents

Representative anesthetic agents that can be administered using the particle formulation described herein include lidocaine, bupivacaine, benzocaine, acetocaine, tetracaine, and prilocaine, among others.

Representative analgesic agents that may be incorporated into the particle formulation include nonsteroidal analgesic agents such as acetaminophen, acetylsalicylic acid, and the anti-inflammatory agents above, as well as opioid analgesics such as buprenorphine, butorphanol, codeine, diamorphine, dihydrocodeine, ethylmorphine, fentanyl, hydrocodone, hydromorphone, isomethadone, levorphanol, lofentanil, meperidine, methadone, morphine, nalbuphine, oxycodone, oxymorphone, pentazocine, propoxyphene, sufentanil and tramadol. As known in the art, there are opioid receptors in bladder tissue (Westerling et al. (2007), “Opioids and Bladder Pain/Function,” in Schmidt et al. (eds.), Encyclopedia of Pain (Springer, Berlin)), and these agents are therefore useful to treat bladder pain syndrome (BPS) and other pain within the urinary system, regardless of the underlying etiology, using the formulation and method of the invention.

### Diuretic Agents

Diuretic agents administrable with the present formulations include, by way of example, loop diuretics such as furosemide, ethacrynic acid, bumetanide, and torasemide; thiazide diuretics such as hydrochlorothiazide and bendroflumethiazide; potassium-sparing diuretics such as spironolactone, epierenone, potassium canreonate, amiloride, and triamterene; and xanthenes such as theophylline and theobromine.

### Biologics:

Biological agents administrable using the particle formulation of the invention include, for example, proteins, peptides, peptide fragments, monoclonal antibodies, enzymes, amino acids, nucleic acids (e.g., DNA, modified DNA, mRNA), cytokines, hormones, and chimeric antigen receptor (CAR) T cell therapy.

Other examples of active agents that can be incorporated into the particle formulation include, RAAS inhibitors for treatment of diabetic nephropathy such as the angiotensin-converting-enzyme (ACE) inhibitors captopril, zofenopril, fosinopril, enalapril, lisinopril, benazepril, and imidapril; antimuscarinic agents for the treatment of overactive bladder, such as darifenacin, hyoscyamine, oxybutynin, tolterodine, solifenacin, trospium, fesoteridine, benztropine mesylate, orphenadrine, procyclidine, and trihexyphenidyl; and β3-adrenergic receptor agonists for the treatment of overactive bladder, such as amibegron, mirabegron, nebivolol, solabegron, and vibegron.

Other active agent groups include bladder relaxant drugs (e.g., for treating incontinence resulting from detrusor muscle overactivity). Relaxant drugs include, for example, oxybutynin, ipratropium, and tricyclic antidepressants such as amitriptyline, imipramine and desipramine; capsaicin, baclofen and other GABAB receptor agonists); and drugs for treating incontinence due to neurologic sphincter deficiency (such as α-adrenergic agonists, β-adrenergic agents, estrogenic agents, and tricyclic antidepressants).

Any of the aforementioned active agents and active agent types can be administered in combination, and any such combinations can be particularly advantageous for the treatment of certain urinary system disorders. For example, in the treatment of urinary tract infections, intravesicular co-administration of an antibiotic agent with an anesthetic agent would both treat the infection and reduce pain. Combinations of two or more antibiotic agents are also contemplated, e.g., an antibiotic with higher solubility and an antibiotic with lower solubility to provide different drug release rates. Examples of suitable combinations include, without limitation, SSD plus lidocaine, SSD with meropenem, or SSD plus silver nitrate. As another example, treatment of a bladder cancer associated with inflammation would benefit from intravesicular co-administration of a chemotherapeutic agent with an anti-inflammatory agent. Additional examples include: administration of an anti-infective agent in combination with delivery of healthy urinary bacteria such as Lactobacillus to treat urinary tract infections; administration of a secondary agent that acts as an oxidizer to increase the potency of the primary active agent, particularly when the primary active agent is an oligodynamic agent; administration of an agent that is released to dissolve organic matter that encrusts the particles, for example, a layered structure where one layer is comprised of active agent, a next layer comprises potassium citrate or allupurinol to dissolve “stone” material, a next layer comprises active agent, and so on; administration of a synergistic agent like urease, which prevents bacteria from changing urine pH, to ensure that the active agent is in its effective pH range; and administration of an agent that is released which facilitates particle dissolution, e.g. lipase, so as to promote active agent release.

Pharmacologically active agents for treating kidney stones can also be therapeutically administered according to the invention. For instance, intravesicular delivery of allopurinol using the present formulation facilitates the break-up of uric acid based stones and prevents the recurrence of stones, as does intravesicular administration of alkalinization agents such as acetazolamide, sodium bicarbonate, potassium citrate, and magnesium citrate, or thiazide and thiazide-like diuretics such as chlorthalidone or indapamide.

In a variation on the embodiment wherein a pharmacologically active agent is administered via the intravesicular route, a diagnostic agent is administered in the same manner, in a particle formulation as provided herein. The diagnostic agent is one that can be identified, quantified, or monitored using conventional imaging equipment. Diagnostic agents can include iodine-based contrast agents (e.g., iopromide, iohexol, iothalamate, ioxaglate, iopramidol, iosimenol, iodixanol, lipiodol, metrizoate, and the like) as used with voiding cystourethrography, intravenous urography, X-ray computed tomography (CT), and other diagnostic techniques; lanthanide-based contrast agents (e.g., dysprosium and gadolinium chelates, particularly gadoversetamide, gadopentetate dimeglumine, gadobutrol, and the like) and superparamagnetic iron oxide contrast agents commonly used in magnetic resonance imaging (MRI); silver-containing and gold-containing contrast agents such as PEGylated (polyethylene glycol-functionalized) silver and gold nanoparticles, proposed for use in CT imaging; microbubble-type contrast agents as used in ultrasonic imaging; and others, as will be appreciated by those of ordinary skill in the art.

### B. Controlled Release Carrier:

The controlled release particles (i.e., formulation particles, nanoparticles or particles) include the pharmacologically active agent in combination with a controlled release carrier. The carrier may be in the form of a matrix in which the active agent is dispersed or embedded. Alternatively, the particles may be of the coated core type, wherein the controlled release carrier is a coating on an active agent-containing core, or wherein the active agent is present in a coating on a core of controlled release carrier. In aspects, matrix-type particles can be advantageous, as well as carrier-coated active agent-containing cores. As explained in Section C, infra, different shapes and structures are contemplated (e.g., particles, tablets, capsules, expandable and collapsible systems, etc.).

The choice of controlled release carrier depends on multiple factors. First, the carrier is selected such that the specific gravity of a particle formulated with a particular active agent and the carrier is lower than that of urine. As the specific gravity of urine is in the range of about 1.005 to 1.03, the specific gravity of a particle formulated with the selected active agent should be less than 1.03, or less than 1.005. Generally, this also means that the specific gravity of the controlled release carrier is less than 1.03, or less than 1.005. The carrier should also provide the desired type of controlled release (i.e., it should be gradually soluble in urine). The carrier should be bioerodible (e.g., by enzymatic activity), physically degradable, or some combination thereof. Hydrophilicity and hydrophobicity are also considerations. Generally, hydrophilic carriers are used with hydrophilic active agents, and hydrophobic carriers with hydrophobic active agents. The controlled release carriers are solid or semi-solid, although viscous liquid carriers can be used providing that suitable particles can be provided therewith in a selected liquid vehicle.

In embodiments, triclycerides are a primary component of the particles. Triglycerides are the most common form of fat in the bloodstream and also found in urine in the bladder. Triglycerides and other lipids are well established as drug carriers and frequently used for implants with a controlled release of active ingredients. Accordingly, triglycerides can be preferred due to their biocompatibility, good safety profile, low toxicity, ease of fabrication and manufacturing, predicates for use in formulations for mucosal surfaces and applications, low density to enhance buoyancy, and hydrophobicity for slow degradation in urine and controlled drug release.

The Applicant has tested several materials for particles compositions including, Dynasan®, Softisan®, Witepsol®. These materials differ by their melting point and hydroxyl value. Materials with lower melting point closer to body temperature (30-39 C) will degrade faster than materials with higher melting points. The hydroxyl value is defined as the number of milligrams of potassium hydroxide (KOH) required to neutralize the acetic acid taken up on acetylation of one gram of a chemical substance that contains free hydroxyl groups. Hydroxyl is a common hydrophilic group, and can be used to modify the surface and change the wettability of the excipient which is owed its ability to form hydrogen bonds with surrounding water. The density of the hydrogen bond formed between hydroxyls and water molecules in the adsorption layer contributes to the overall wettability of the excipient. Increase in wettability can increase degradation of excipients via hydrolysis and thereby enable drug release. Both properties can be considered to select the base, predominant excipient for formulation or combination of several excipients to achieve the ideal degradation and drug release profile and rate.

In aspects, the main polymer material includes a hydrophobic material with a high melting point such as glyceryl tripalmitate (e.g., Dynasan 116®) or glyceryl tristearate (Dynasan 118®), glyceryl monostearate (Dynasan 114®) at range of 50%-95% (w/w) mixed with an excipient at relative range of 1%-50% (w/w) to enhance degradation and drug release. The polymer can be melted and then mixed with drug (or API) at range of 1%-25% (w/v). Additional materials that have been tested for profiling degradation rate, drug release, and compatibility with other excipients are listed below.

Other representative carrier materials suitable for forming the particles herein include, for example, the following:
(1) Saturated C₆-C₃₀ fatty acids such as caproic acid, enanthic acid, caprylic acid, capric acid, caproic acid, pelargonic acid, undecylic acid, lauric acid, myristic acid, palmitic acid, stearic acid, arachidic acid, behenic acid, cerotic acid, montanic acid, and nonacosylic acid, as well as salts thereof, e.g., sodium caprylate, sodium laurate, sodium myristate, and the like;
(2) Unsaturated C₆-C₃₀ fatty acids such as crotonic acid, myristoleic acid, palmitoleic acid, oleic acid, eicosenoic acid, linoleic acid, docosadienoic acid, eicosadienoic acid, and linolenic acid;
(3) Saturated or unsaturated C₁₀-C₃₀ fatty alcohols, typically monohydric fatty alcohols, such as undecanol, lauryl alcohol, myristyl alcohol, cetyl alcohol, palmitoleyl alcohol, stearyl alcohol, oleyl alcohol, nonadecyl alcohol, behenyl alcohol, cetyl alcohol, and myricyl alcohol;
(4) Fatty acid esters, i.e., typically derived from a monohydric, dihydric, or polyhydric alcohol and at least one C₈-C₃₀ fatty acid, including:
(a) lower alcohol fatty acid esters, i.e., esters of lower (C₂-C₆) alcohols and fatty acids, such as ethyl oleate, isopropyl myristate, and isopropyl palmitate;
(b) triglycerides such as glyceryl tributyrate, glyceryl tricaproate, glyceryl tricaprylate, glyceryl tricaprate, glyceryl triundecanoate, glyceryl trilaurate, glyceryl trimyristate, glyceryl tripalmitate, glyceryl tristearate, glyceryl trimyristoleate, glyceryl trioleate, and mixtures thereof such as glyceryl tricaprylate/caprate/laurate, glyceryl tricaprylate/caprate/linoleate, glyceryl tricaprylate/laurate stearate, and the like;
(c) mono- and diglycerides such as glyceryl ricinoleate, glyceryl laurate, glyceryl dilaurate, glyceryl monooleate, glyceryl dioleate, glyceryl monostearate, glyceryl behenate (e.g., Compritol®, Gattefosse), glyceryl distearate, glyceryl monopalmitate, glyceryl palmitostearate (e.g., Precirol®, Gattefosse), glyceryl caprylate/caprate (Capmul®, Abitec), caprylic acid mono and diglycerides (e.g., Imwitor 998), and mono- and di-acetylated monoglycerides (e.g., Myvacet 9-45);
(d) polyglycerized fatty acids, i.e., polyglycerol esters of fatty acids such as polyglyceryl-2 stearate, polylglyceryl-2 oleate, polyglyceryl-2 isostearate, polyglyceryl-6-oleate, polyglyceryl-10 laurate, polyglyceryl-10 laurate, polyglyceryl-10 stearate, polyglyceryl-6 dioleate, polyglyceryl-10 trioleate, polyglyceryl polyricinoleates, tetraglyceryl pentastearate, and tetraglyceryl monostearate;
(e) propylene glycol fatty acid esters, i.e., esters of propylene glycol and fatty acids, such as propylene glycol monolaurate, propylene glycol ricinoleate, propylene glycol monooleate, propylene glycol dioctanoate, propylene glycol stearate, and propylene glycol myristate;
(f) polyethoxylated fatty acids, i.e., polyethylene glycol (PEG) fatty acid esters such as PEG-1 stearate, PEG-2 stearate, PEG-2 oleate, PEG-4 laurate, PEG-4 oleate, PEG-4 stearate, PEG-6 laurate, PEG-6-oleate, PEG-6 stearate, PEG-8 laurate, PEG-8 oleate, PEG-8 stearate, PEG-10 laurate, PEG-10 oleate, PEG-10 stearate, PEG-12 laurate, PEG-12 oleate, PEG-12 ricinoleate, PEG-200 oleate, PEG-400 oleate, PEG-4 dilaurate, PEG-4 dioleate, PEG-4 distearate, PEG-10 dipalmitate, PEG-8 dilaurate, PEG-8 dioleate, PEG-8 distearate, PEG-10 dipalmitate, PEG-12 dilaurate, PEG-20 distearate, PEG-32 distearate, and PEG-400 distearate;
(g) polyethoxylated glycerol fatty acid esters, i.e., compounds wherein two of the three glycerol hydroxyl groups are esterified with a fatty acid and the third bears a PEG substituent, such as PEG-20 glyceryl laurate, PEG-30 glyceryl laurate, PEG-40 glyceryl laurate, PEG-20 glyceryl stearate, PEG-20 glyceryl oleate, and PEG-30 glyceryl oleate; and
(h) sorbitan fatty acid esters, such as sorbitan monolaurate, sorbitan monopalmitate, sorbitan monooleate, sorbitan monostearate, sorbitan tristearate, sorbitan sesquioleate, and sorbitan trioleate, and polyethoxylated sorbitan fatty acid esters, such as PEG-20 sorbitan monolaurate (Tween-20), PEG-20 sorbitan monopalmitate (Tween-40), PEG-20 sorbitan monostearate (Tween-60), and PEG-20 sorbitan monooleate (Tween-80);
(i) diesters formed from a C₈-C₃₀ fatty acid and a C₈-C₃₀ fatty alcohol and a carboxylic acid bearing a hydroxyl group, such as 4-hydroxycinnamic acid (coumaric acid) and w-hydroxy acids such as 16-hydroxy palmitic acid, 18-hydroxy stearic acid, and the like;
(5) phospholipids such as phosphatidylcholine, phosphatidylethanolamine, phostatidylinositol, phosphatidylserine, phosphatidyl glycerol, phosphorylated diacyl glycerides, particularly phospholipids selected from diacyl phosphatidylcholines, diacyl phosphatidylethanolamines, diacyl phosphatidylserines, diacyl phosphatidylinositols, diacyl phosphatidylglycerols, diacyl phosphatidic acids, and mixtures thereof, wherein each acyl group contains about 10 to about 30 carbon atoms and is saturated or unsaturated, and phospholipid mixtures such as lecithin, hydroxylated lecithin, and lysolesithin;
(6) sterols (including sterol derivatives) such as cholesterol, sitosterol, lanosterol, PEG-24 cholesterol ether, PEG-30 cholestanol, and phytosterol;
(7) polyethylene glycol alkyl ethers, i.e., ethers of PEG and aliphatic alcohols, such as PEG-3 oleyl ether and PEG-4 lauryl ether;
(8) polyoxyethylene-polyoxypropylene (POE-POP) block copolymers, i.e., hydrophilic surfactants with varying POE-POP ratios, typically referred to as poloxamers, and including poloxamer 105, Po108, poloxamer 122, poloxamer 123, poloxamer 124, poloxamer 181, poloxamer 182, poloxamer 183, poloxamer 184, poloxamer 185, poloxamer 188, poloxamer 212, Pod 215, poloxamer 217, poloxamer 231, poloxamer 234, poloxamer 235, poloxamer 237, poloxamer 238, poloxamer 282, poloxamer 284, poloxamer 288, poloxamer 331, poloxamer 333, poloxamer 334, poloxamer 335, poloxamer 338, poloxamer 401, poloxamer 402, poloxamer 403, and poloxamer 407, as well as combinations of the foregoing and combinations with other compounds. e.g., poloxamer 407 (45%) and ammonium bicarbonate (6%). The latter combination is advantageous insofar as ammonium bicarbonate decomposes at elevated temperatures to give carbon dioxide bubbles, which in turn facilitate flotation of the particle formulation in the bladder.
(9) gradually erodible or degradable synthetic polymers such as polylactides, polyglycolides, polycaprolactones, polyanhydrides, polyurethanes, polyesteramides, polyorthoesters, polyvinylpyrrolidone, polyhydroxycellulose, and copolymers, derivatives, and mixtures thereof;
(10) hydrophilic polymers typically used to prepare hydrogels, e.g., cellulosic polymers such as hydroxypropyl cellulose, hydroxyethyl cellulose, hydroxypropyl methyl cellulose, methyl cellulose, ethyl cellulose, cellulose acetate, and sodium carboxymethylcellulose (NaCMC); acrylic acid polymers such as methacrylic acid, acrylic acid alkyl esters, methacrylic acid alkyl esters, and the like; copolymers of acrylic acid, methacrylic acid, acrylic acid alkyl esters, methacrylic acid alkyl esters, and the like, such as copolymers of acrylic acid, methacrylic acid, methyl acrylate, ethyl acrylate, methyl methacrylate, ethyl methacrylate, and/or hydroxyethyl methacrylate; vinyl polymers and copolymers such as polyvinyl alcohol, polyvinyl pyrrolidone, polyvinyl acetate, and ethylene-vinyl acetate copolymer; and chitosan;
(11) bile salts such as sodium cholate, sodium deoxycholate, and the like; and
(12) surfactants not encompassed by the above groups, such as sodium lauryl sulfate, cetyltrimethyl-ammonium bromide, benzalkonium chloride, 2-phenoxyethanol, and benzoyl alcohol.

It will be appreciated that the foregoing examples of controlled release carriers that can be used in the present particles include hydrophilic materials, hydrophobic materials, surfactants, naturally occurring materials, synthetically modified naturally occurring materials, synthetic materials; and materials having molecular weights within a relatively wide range.

### Mucoadhesive Controlled Release Materials:

Direct intravesical instillation of therapeutic drugs are still limited by constant dilution with urine production and elimination by voiding. These limitations necessitate frequent and repeated administration, which reduces patient compliance and increases cost of treatment. Formulations that use mucoadhesive materials could potentially increase the drug residence time in the bladder by adhering to the bladder mucosa and endure washout by urination. This can ultimately enhance bioavailability and effectively reduce instillation frequency and dose required for therapeutic potency. Mucoadhesion to the bladder inner wall can be achieved via electrostatic interaction with the mucosa with cationic materials, or materials that form hydrogen and covalent bonds with the bladder mucosal tissues.

Controlled release carriers can include a mucoadhesive material to enhance retention of the formulation within the bladder (i.e., by adherence to the bladder wall). That is, use of a mucoadhesive controlled release carrier can extend the time period during which the particle formulation is present within the bladder and thereby extend the duration of drug delivery as well. Any mucoadhesive material can be used, including mucoadhesive materials within groups (1) through (12), above, providing that the desired degree of retention is achieved, that the desired level of particle buoyancy in urine is provided, and that the desired sustained release profile results. Ideally, the material provides for approximately zero order release, i.e., the active agent is released within the bladder at an approximately constant release rate.

Suitable mucoadhesive materials for use in or as the controlled release carrier herein, facilitating particle buoyancy, retention, and sustained release, include the following:
(1) Waxes, particularly paraffin wax, candelilla wax, beeswax, and carnauba wax; Fatty acid esters derived from a monohydric, dihydric, or polyhydric C₂-C₆ alcohol and a C₈-C₂₄ fatty acid, e.g., monoglycerides, diglycerides, and triglycerides of C₈-C₂₂ saturated fatty acids, with representative examples including triglycerides such as glyceryl tripalmitate (such as that available under the tradename Compritol® from Gattefossé), blends of triglycerides of C₈-C₂₂ saturated fatty acids (such as Softisan® 378, from IOI Oleo); blends of a triglyceride of a C₈-C₂₂ saturated fatty acid and a monoglyceride of a C₈-C₂₂ fatty acid (such as Witepsol®) H15); blends of mono- and di-glycerides of two or more C₈-C₂₂ saturated fatty acids (such as glyceryl palmitostearate, a blend of glycerol palmitate, glyceryl stearate, and the diglyceride with both fatty acids); and monoglycerides of C₈-C₂₂ saturated fatty acids, particularly glyceryl behenate;
(2) Fatty acids, particularly C₈-C₂₄ fatty acids. e.g., saturated C₈-C₁₈ fatty acids, saturated C₁₂-C₂₄ fatty acids, and saturated C₈-C₂₂ fatty acids, such as palmitic acid and stearic acid;
(3) Fatty alcohols, particularly C₈-C₂₄ fatty alcohols, e.g., saturated C₈-C₁₈ fatty alcohols, saturated C₁₂-C₂₄ fatty alcohols, and saturated C₈-C₂₂ fatty alcohols, such as stearyl alcohol and cetyl alcohol;
(4) Hydroxylated C₄-C₁₂ alkanes, including polyhydroxylated and dihydroxylated C₄-C₁₂ alkanes, such as 1,2-octanediol, 1,2-hexanediol, 1,2-pentanediol, and 1,3-butanediol;
(5) Polymers and copolymers of hydroxylated vinyl monomers, such as poly(vinyl alcohol);
(6) Polymers and copolymers of carboxylated vinyl monomers such as poly(acrylic acid), crosslinked poly(acrylic acids) (carbomers), poly(methacrylic acids), copolymers of methylvinyl ether with acrylic acid and/or methacrylic acid, and the like, particularly carbomers and modified derivatives thereof sold under trademark Carbopol®, such as Carbopol 71G NF, Carbopol 934 NF, Carbopol 934P NF, Carbopol 940 NF, Carbopol 941NF, Carbopol 971P NF, Carbopol 974P NF, Carbopol 980 NF, Carbopol 981 NF, Carbopol 1342 NF, Carbopol 5984 EP, Noveon AA-1 Polycarbophil, Carbopol Ultrez 10NF, Carbopol Ultrez 20, Carbopol Ultrez 21, Carbopol ETD 2020 NF, Pemulen TR1 NF, Pemulen TR2 NF, and others, commercially available from Lubrizol Advanced Materials, Inc.
(7) Cellulosic polymers, including carboxy celluloses such as sodium carboxymethyl cellulose and hydroxyalkyl celluloses such as hydroxymethyl cellulose, hydroxypropyl cellulose, and hydroxyethyl cellulose.

It will be appreciated that other mucoadhesive materials that provide the necessary buoyancy, retention, and release profile may be used as alternatives to or in addition to any of the foregoing:

### Compositions/Ratio of Components

The ratio of the active agent to the controlled release carrier in the formulation, as will be appreciated by those in the field of drug delivery, depends on the active agent and the intended dose to be administered, which in turn depends on the drug delivery time period. In general, the weight ratio of active agent to controlled release carrier is selected to provide drug loading (weight percent of active in the particles) in the range of about 5% to 50% (corresponding to a weight ratio range of 1:20 to 1:1), e.g., 10% to 50% (corresponding to a weight ratio range of 1:10 to 1:1), usually in the range of 20% to 50% (corresponding to a weight ratio range of 1:5 to 1:1), and most typically in the range of 25% to 40% (corresponding to a weight ratio range of 1:3 to 2:3).

In general, the particles completely dissolve, degrade, or erode within the bladder, so removal of the formulation is unnecessary. The particle formulation has an intravoid retention rate of less than 1, meaning that some fractional loss of particles occurs with each void. In some cases, it might be desirable to facilitate particle degradation or removal prior to the end of the intended drug delivery period. The particle formulation can be removed by suction or by introduction of a particle degrading agent into the bladder. The degrading agent can be an enzyme, a chemical reagent such as hydrogen peroxide, or a urine acidifier such as methenamine hippurate.

In some embodiments, the particles are coated with or contain one or more enzymes or other biofilm disrupting agents, selected so as to degrade the extracellular matrix of a biofilm, e.g., dispersin B, trypsin (or other digestive enzymes), deoxyribonuclease, nitric oxide, etc. A particle coating or component can also include materials that decrease the affinity of crystals present in the urine from attaching to the particle surface (e.g., polyurethane or polytetrafluoroethylene) or that using an electrostatically charged material to adhere to the biomaterials (e.g., acrylamidotaurate) and cause destabilization. Any of these mechanisms for biofilm disruption and prevention of further biofilm growth can be used in combination, and some may work synergistically.

### C. Particle Fabrication:

### Controlled Release Matrix Particles

The particles herein having a pharmacologically active agent dispersed within a matrix of a controlled release carrier can be fabricated as follows. The selected controlled release carrier is first melted, and the active agent is then added to the melted carrier to provide an active agent-carrier admixture. The admixture is homogenized using any suitable equipment that facilitates substantially uniform distribution of the active agent in the carrier, and heating may be carried out simultaneously to ensure that the admixture remains in the form of a melt. Following at least several minutes of homogenization, the homogenized, melted admixture is fed into an atomizer so as to generate droplets of the admixture, which congeal in flight to provide the desired particles, which are then collected. Rotary atomizers are typically preferred. It will be appreciated that the particle size can be varied by controlling the feed material viscosity (for instance by adjusting the temperature of the melt), the rotational speed of the atomizer, the feed rate, and the size of the rotary disk. A representative protocol is described in Example 3. Particle size is inversely proportional to feed material viscosity, rotational speed, and feed rate. Accordingly, for larger particles, one can decrease feed material viscosity, rotational speed, and/or feed rate, while to obtain smaller particles, a lower feed material viscosity, rotational speed and/or feed rate is necessary.

Particles of the coated core type, wherein the controlled release carrier is provided as a coating on an active agent-containing core, or wherein the active agent is in a coating provided on a core that includes the controlled release carrier, may be fabricated using conventional methods known to those of ordinary skill in the art and/or described in the pertinent texts and literature. See, for example, the description of encapsulated sustained release delivery systems in Remington: The Science and Practice of Pharmacy, 19th Ed., Vol. 2 (Mack Publishing Co., 1995), at Ch. 34.

The particles may be fabricated so as to be at least somewhat porous, using techniques known in the art. Gas bubbles or air bubbles may be introduced into the matrix or coating, if present. Alternatively, or in addition, a porogen (e.g., a poloxamer) can be incorporated into a controlled release coating, or, for controlled release matrix-type particles, into the melt prior to, during, or after the particles are dispersed therein. See Cai et al. (2013), “Porous microsphere and its applications,” Int J Nanomedicine 8:1111-1120, for additional information on the preparation of porous microspheres, incorporated herein by reference.

The controlled release particles may also be fabricated using a hot melt extrusion technique. Hot melt extrusion (HME) is well known in the art of manufacturing polymer-based formulations and dosage forms and is described in the pertinent texts and literature. See, e.g., Simoes et al. (2021), “Hot-Melt Extrusion: a Roadmap for Product Development,” AAPS PharmSciTech 22 (5) 184; and Chivate et al. (2021), “Hot-Melt Extrusion: An Emerging Technique for Solubility Enhancement of Poorly Water-Soluble Drugs,” PDA J Pharm Sci Technol. 75 (4): 357-373, both incorporated by reference herein. Hot melt extrusion is particularly useful, although not limited to, instances in which active agents and/or a carrier or excipient is poorly soluble or unstable using other manufacturing techniques.

### Shape/Form

Preparation methods for lipidic drug carriers include direct compression, solid lipid extrusion, and melting techniques. To formulate different shapes and geometries of drug carrier, manufacturing techniques such as injection molding, melt-spray congeal, compression, solid lipid extrusion via hot melt extrusion, and casting technique using 3D printed molds were employed. The final form that includes cylindrical rods with diameter ranging from 2-5 mm and height of 5-15 mm were formulated with melting polymer with drug and injecting in a silicone tube mold of desirable size. Spheres varying in diameter size from 100 μm-1 mm were formulated using melt spray congeal with atomizer. Spheres above 1 mm in diameter were formulated by casting hot melt mix of polymer and drug in 3D printed molds.

Although it would be conventionally assumed that controlled release particles are substantially spherical in shape, this is not necessarily the case. The particles of the invention may be spherical or substantially spherical, but the invention is not limited in this regard, insofar as the particles may have any three-dimensional structure that results from the selected fabrication techniques, providing that they are generally of a size and shape that do not cause obstruction of the urethra or of the catheter used in the intravesicular administration of the formulation. The particles may be cylindrical, spherocylindrical, oval, ovoid, etc., or a combination of any of the foregoing, i.e., polymorphous.

The particles are administered as a controlled release pharmaceutical formulation for intravesicular administration to a subject, such that the formulation comprises a population of particles. Substantially spherical particles in the formulation have a mean diameter of greater than 2.0 mm to about 8.0 mm, e.g., 2.5 mm to 6.5 mm. e.g., 2.5 mm to 6.0 mm, 2.5 mm to 5.5 mm, 2.5 mm to 5.0 mm, 2.5 mm to 4.5 mm, 2.5 mm to 3.5 mm, or 2.0 mm to 3.0 mm. For nonspherical particles, the analogous measurement is the length of the longest dimension of the particle (and “diameter” is used generically herein to encompass that measurement). As noted herein, the particle size distribution in the population of particles in the formulation administered to the subject is relatively narrow. i.e., the particles should be within 20%, preferably within 10%, of the median particle diameter. For example, if the median diameter is 4 mm, the particle size range should be between 3.2 mm and 4.8 mm (within 20% of the median diameter), preferably between 3.6 mm and 4.4 mm (within 10% of the median diameter.

A significant feature of the particles is their buoyancy. In one embodiment, all or substantially all of the particles in the particle population are buoyant in urine. This allows the particles to float to the surface of urine in the bladder, significantly reducing the fraction of the particle formulation that will be released with each emptying of the bladder. Buoyancy in urine is achieved by the fabrication of particles having a specific gravity that is generally less than 1.03, more typically less than 1.005, insofar as the specific gravity of urine is in the range of about 1.005 to 1.03. This in turn corresponds, typically, to the use of a controlled release carrier having a specific gravity that is generally less than 1.03, more typically less than 1.005.

The size and buoyancy of the particles provide benefits specific to the bladder. For example, in certain embodiments, the size and/or shape of the particles allows for passage through the urethra, when needed, to prevent blockage. In addition to the mucoadhesive carrier, the buoyancy of the particles also aids in retention of the particles in the bladder regardless of any forces acting on the particle by the bladder walls during voiding of the bladder. The retention of the particles also benefits patients since the particles fill any post-void residual space of the contracted bladder, which displaces any urine that would otherwise remain within the bladder and possibly exacerbate an infection, e.g., by facilitating bacterial or fungal growth. If the contracted bladder contains excess particles, the size of the particles allows them to pass through the urethra to avoid any increase in the frequency of urination. Finally, as explained earlier herein, the contact between the particles and the interior walls of the bladder or urethra, as well as particle movement within the bladder causing turbulence in urine, substantially prevents biofilm formation and facilitates mechanical disruption of any biofilm already present, in turn reducing the likelihood of adherence of bacterial cells to a biofilm matrix.

The particles in the formulation may also be designed to coalesce within the bladder. For instance, in one representative example of such a formulation, the particles may be hemispherical, coalescing plane to plane to provide particle spheres within the bladder.

The formulation may comprise a first subset of particles and a second subset of particles, wherein the particles in the two subsets differ in some way, e.g., in mean diameter, shape, buoyancy, deformability, or in two or more of such properties. Accordingly, in one embodiment, the first and second particle subsets differ in size, such that the particles in one subset are relatively large particles of a non-tiling shape (i.e., a shape that prevents formation of a voidless layer), e.g., with diameter or longest dimension in the range of 2.0 mm to about 8.0 mm, e.g., 2.0 mm to about 6.0 mm or 2.0 mm to 4.5 mm and the smaller particles in the range of about 0.5 mm to 2.0 mm the second subset can pass through the voids left by the non-tiled larger particles. In another embodiment, the formulation includes particles having different levels of buoyancy, such that one subset of particles is quite buoyant and the second subset is less buoyant, particles within the formulation may also differ in both size and buoyancy.

According to another embodiment, the first subset of particles, the second subset of particles, or both the first and second subsets of particles are reversibly deformable. For instance, the first subset of particles, the second subset of particles, or both the first and second subsets of particles are deformable to an extent that allows flow within a chute having an inner diameter in the range of 6 mm to 8 mm.

Particles within the formulation may, in addition, or in the alternative, include particles having different pharmacologically active agents, different amounts of the same or a different pharmacologically active agent, different controlled release carriers, different amounts of controlled release carrier, have a different controlled release profile, or any combination of the foregoing.

### D. Vehicle/Excipients

The formulation administered to a subject to treat a urinary system disorder includes the population of controlled release particles described in the preceding section, and generally includes at least one pharmaceutically acceptable excipient, i.e., a formulation component without pharmacological activity that imparts a desired physical or chemical property to the formulation or to a component thereof.

The population of particles may be incorporated into a sterile liquid vehicle, which may be aqueous or nonaqueous, such that the formulation comprises an aqueous or nonaqueous dispersion, suspension, or emulsion of the particles in the liquid vehicle. Examples of nonaqueous liquid vehicles include fatty oils, which, it will be appreciated, comprise mixtures of fatty acids, fatty acid diglycerides, and/or fatty acid triglycerides, such as castor oil, cottonseed oil, corn oil, linseed oil, mineral oil, olive oil, sesame oil, soybean oil, and the like; fatty acids that are liquid at room temperature, i.e., lower molecular weight and/or unsaturated fatty acids, e.g., oleic acid, linoleic acid, and linolenic acid; alcohols such as such as propylene glycol, glycerol, and lower molecular weight polyethylene glycol (molecular weight less than about 750 g/mol). Ideally, the liquid vehicle should have a viscosity in the range of 2 cP to 400 cP.

The formulation may also contain, in addition to the particles and any liquid vehicle containing the particles, excipients such as buffers and other pH-adjusting agents, viscosity adjusting agents, dispersants, tonicity adjusting agents, enzyme inhibitors, preservatives and stabilizers, solubilizers, and emulsifiers.

A pH adjusting agent should maintain the pH of the formulation in the range of 5.8 to 7.4 so as not to affect the local pH of urine. Representative pH adjusting agents that serve this purpose include phosphate buffered saline (PBS) and other phosphate buffers, such as monobasic potassium phosphate, dibasic potassium phosphate, and pyrophosphate buffers; bicarbonates such as sodium bicarbonate; histidine/histidine hydrochloride; citrates such as disodium citrate and trisodium citrate; acetates such as ammonium acetate; tris(hydroxymethyl) aminomethane (Tris) buffers, arginine; and meglumine. The pH adjusting agent additionally serves to prevent crystallization of any solute on the particles.

Viscosity adjusting agents are thinners or thickeners, although in the present formulation any viscosity-adjusting agent is typically a thickener. Viscosity-adjusting agents herein facilitate transport of the formulation through the selected intravesicular delivery system (e.g., a catheter, intraurethral syringe, etc.) and are selected to maintain a formulation viscosity in the range of 2 cP to 400 cP. Suitable viscosity adjusting agents include sodium carboxymethyl cellulose (NaCMC), sorbitol, dextran, acacia, gelatin, methylcellulose, and poly(vinylpyrrolidone). As an example, for NaCMC to maintain formulation viscosity in the aforementioned range, this generally means using a CMC with a molecular weight in the range of 90 kDa to 700 kDa, at a concentration in the range of 5 to 30 mg/mL, e.g., 5 mg/mL, 10 mg/mL, 20 mg/mL, or 30 mg/mL. Molecular weight and concentration are, of course, to be taken into account with any viscosity adjusting agent, and these parameters can be selected (molecular weight) or varied (concentration) as necessary to achieve the target viscosity. A higher molecular weight viscosity adjusting agent will result in a more viscous solution, as will a higher concentration (e.g., 5 mg/mL of 250 kDa CMC provides a viscosity similar to that obtained with 20 mg/ml of 90 kDa CMC).

### Dispersants

The purpose of the dispersant is to prevent clumping and sticking of the particles and facilitate dispersion of the particles in the formulation. Dispersants, as is well known in the art, are typically, although not necessarily, surfactants; exemplary surfactants herein include the poloxamers identified above, and particularly Tween 20 and Tween 80, incorporated into the formulation in an amount ranging from 0.1 mg/ml to 5 mg/ml, e.g., 0.1 mg/mL, 0.5 mg/mL, 1.0 mg/mL, and 5 mg/mL (0.01%, 0.5%, 0.1%, and 0.5%, respectively). Another dispersant of interest herein is poly(vinylpyrrolidone) (PVP), e.g., Povidone K12 and Povidone K17.

Tonicity adjusting agents are incorporated to render the formulation isotonic in urine. Commonly used tonicity adjusting agents may be used herein, and include dextrose, glycerol, D-mannitol, and sodium chloride. Exemplary tonicity adjusting agents that can be incorporated into the present formulation are D-mannitol, sodium chloride, and combinations thereof; an aqueous solution with 5% D-mannitol and 0.9% sodium chloride renders the solution isotonic in urine.

Another excipient group includes enzyme inhibitors, including inhibitors of enzymes that might degrade the pharmacologically active agent, inhibitors of enzymes present within the bladder or other region of the urinary system at a pathological level; and urease inhibitors. Insofar as urease is a known virulence factor for some urinary tract pathogens (including S. saprophyticus and P. mirabilis), incorporation of a urease inhibitor into the particle formulation can limit bacterial growth. Furthermore, bacterial urease alkalizes urine, causing supersaturation of calcium phosphate and struvite, consequent crystal formation, and potentially generation of a urinary stone.

Representative preservatives include antioxidants, antimicrobial agents, and chelating agents. An antioxidant preservative is useful in minimizing oxidation of the pharmacologically active agent or any excipient over the shelf life of the formulation. Commonly used antioxidant preservatives that may be advantageously used herein include ascorbic acid, ascorbyl palmitate, sodium ascorbate, acetylcysteine, monothioglycerol, and sulfurous acid salts (bisulfites, metabisulfites, and the like). Antimicrobial preservatives for preventing growth of micro-organisms in the formulation during storage and prior to use include agents such as benzalkonium chloride, benzyl alcohol, methyl paraben, propyl paraben, and thimerosal. Chelating agents such as ethylenediaminetetraacetic acid (EDTA) and salts thereof (disodium EDTA, tetrasodium EDTA, sodium calcium edetate, etc.) are useful to sequester metal ions in the formulation that could otherwise facilitate unwanted enzymatic or other reactions.

If the formulation is an emulsion, as may be the case with hydrophobic particles in a hydrophilic vehicle, hydrophilic particles in a hydrophobic vehicle, a mixture of hydrophilic and hydrophobic particles, or a mixture of hydrophilic and hydrophobic excipients, use of an emulsifier is recommended to facilitate homogeneous dispersion of particles and/or excipients in a liquid vehicle. Emulsifiers, as known in the art, are surfactants, comprised of a polar or charged hydrophilic moiety and a non-polar lipophilic (hydrophobic) moiety. Emulsifiers herein can also serve as a dispersant, stabilizer, and/or the liquid vehicle. Suitable emulsifiers for incorporation into the present formulation include any emulsifiers that are typically used in non-solid pharmaceutical preparations, such as poloxamers (including those identified above), polyoxyethylene ethers, polyethoxylated castor oil, polyoxyethylene fatty acid esters (polysorbates), polyoxyethylene stearates, propylene glycol alginate, sodium citrate, sorbitan fatty acid esters, lecithin, and diethanolamine. See, e.g., Handbook of Pharmaceutical Excipients, 3rd Edition, Kibbe, ed. (American Pharmaceutical Association, 2000).

### E. Particle Combinations

The intravesicular formulation can include two or more populations of particles, with each population differing from each other population in at least one respect. In another embodiment, then, the formulation additionally comprises a secondary population of particles that differs from the primary population of particles, for example, comprising a different pharmacologically active agent, a different amount of a pharmacologically active agent, a different controlled release carrier, or a different amount of controlled release carrier, or having a different controlled release profile or specific gravity.

For example, a first, or primary, population of particles may provide a first drug release profile (e.g., substantially immediate release of a bolus dose) and second, or secondary, population of particles may provide a second drug release profile different from the first (e.g., a sustained release profile). As another example, one population of particles can contain a first pharmacologically active agent and a second population of particles can contain a second pharmacologically active agent, and the two populations may or may not be formulated so as to have different drug release profiles. In some cases, it may be desirable that some fraction of the particles administered to the bladder sink to the trigone and exhibit the intended pharmacological activity there—for example to disrupt a biofilm at the trigone, to deliver an anesthetic agent to the trigone, or to administer an anti-infective agent to the trigone—an additional population of particles can be fabricated so as to be less buoyant than the first population. As alluded to earlier herein, this can be achieved by selecting a different controlled release carrier, a different carrier-to-active agent ratio, or the like. Inclusion of less buoyant particles in the formulation can assist in delivery of an active agent to regions of the bladder that retain urine after contraction, for instance a cystocele (also known as a prolapsed, herniated, dropped, or fallen bladder). With a cystocele, the ligaments that hold the bladder and surrounding muscles stretch or weaken, allowing the bladder to sag, and it is the sagging region that can be treated by deposition of the less buoyant or nonbuoyant particles in that area.

### F. Representative Formulations and Dosage Considerations:

Representative particle formulations according to the invention are as follows:
5 wt. % SSD, 95 wt. % candelilla wax;
10 wt. % SSD, 90 wt. % candelilla wax;
15 wt. % SSD, 85 wt. % candelilla wax;
25 wt. % SSD, 75 wt. % candelilla wax;
40 wt. % SSD, 60 wt. % candelilla wax;
5 wt. % SSD, 95 wt. % paraffin wax;
10 wt. % SSD, 90 wt. % paraffin wax;
15 wt. % SSD, 85 wt. % paraffin wax;
25 wt. % SSD, 75 wt. % paraffin wax;
15 wt. % SSD, 85 wt. % paraffin wax;
25 wt. % SSD, 75 wt. % paraffin wax;
40 wt. % SSD, 60 wt. % paraffin wax;
5 wt. % SSD, 95 wt. % glyceryl tripalmitate;
10 wt. % SSD, 90 wt. % glyceryl tripalmitate;
15 wt. % SSD, 85 wt. % glyceryl tripalmitate;
25 wt. % SSD, 75 wt. % glyceryl tripalmitate;
40 wt. % SSD, 60 wt. % glyceryl tripalmitate;
5 wt. % SSD, 95 wt. % sodium carboxymethylcellulose;
10 wt. % SSD, 90 wt. % sodium carboxymethylcellulose;
15 wt. % SSD, 85 wt. % sodium carboxymethylcellulose;
25 wt. % SSD, 75 wt. % sodium carboxymethylcellulose;
40 wt. % SSD, 60 wt. % sodium carboxymethylcellulose;
5 wt. % SSD, 95 wt. % stearyl alcohol;
10 wt. % SSD, 90 wt. % stearyl alcohol;
15 wt. % SSD, 85 wt. % stearyl alcohol;
25 wt. % SSD, 75 wt. % stearyl alcohol;
40 wt. % SSD, 60 wt. % stearyl alcohol;
5 wt. % SSD, 95 wt. % cetyl alcohol;
10 wt. % SSD, 90 wt. % cetyl alcohol;
15 wt. % SSD, 85 wt. % cetyl alcohol;
25 wt. % SSD, 75 wt. % cetyl alcohol;
40 wt. % SSD. 60 wt. % cetyl alcohol;
5 wt. % SSD, 95 wt. % polyvinyl alcohol;
10 wt. % SSD, 90 wt. % polyvinyl alcohol;
15 wt. % SSD. 85 wt. % polyvinyl alcohol;
25 wt. % SSD. 75 wt. % polyvinyl alcohol;
40 wt. % SSD. 60 wt. % polyvinyl alcohol;
5 wt. % SSD, 95 wt. % 1,2-octanediol;
10 wt. % SSD, 90 wt. % 1,2-octanediol;
15 wt. % SSD, 85 wt. % 1,2-octanediol;
25 wt. % SSD. 75 wt. % 1,2-octanediol;
40 wt. % SSD, 60 wt. % 1,2-octanediol;
10 wt. % SSD. 45 wt. % glyceryl tristearate, 45 wt. % hydroxypropyl methylcelulose (HPMC);
20 wt. % SSD. 40 wt. % glyceryl tristearate, 40 wt. % HPMC;
40 wt. % SSD, 30 wt. % glyceryl tristearate, 30 wt. % HPMC;
15 wt. % SSD. 65 wt. % glyceryl tristearate, 10 wt. % stearyl alcohol;
20 wt. % SSD. 70 wt. % glyceryl tristearate, 10 wt. % stearyl alcohol;
15 wt. % SSD. 65 wt. % glyceryl tripalmitate, 10 wt. % stearyl alcohol;
20 wt. % SSD. 70 wt. % glyceryl tripalmitate, 10 wt. % stearyl alcohol;
15 wt. % SSD. 5 wt. % lidocaine, 80 wt. % candelilla wax;
20 wt. % SSD. 5 wt. % lidocaine, 75 wt. % candelilla wax;
15 wt. % SSD. 5 wt. % lidocaine, 80 wt. % paraffin wax;
20 wt. % SSD. 5 wt. % lidocaine, 75 wt. % paraffin wax;
15 wt. % SSD. 5 wt. % lidocaine, 80 wt. % glyceryl tripalmitate;
20 wt. % SSD. 5 wt. % lidocaine, 75 wt. % glyceryl tripalmitate;
15 wt. % SSD. 5 wt. % lidocaine, 70 wt. % glyceryl tristearate, 10 wt. % stearyl alcohol;
15 wt. % SSD. 5 wt. % lidocaine, 70 wt. % glyceryl tripalmitate, 10 wt. % stearyl alcohol;
15 wt. % SSD. 85 wt. % Softisan® 378 triglyceride blend;
25 wt. % SSD. 75 wt. % Softisan® 378 triglyceride blend;
40 wt. % SSD, 60 wt. % Softisan® 378 triglyceride blend;
10 wt. % SSD, 90 wt. % Witepsol H15;
20 wt. % SSD, 80 wt. % Witepsol H15;
40 wt. % SSD, 60 wt. % Witepsol H15;
5 wt. % silver nitrate, 95 wt. % candelilla wax;
10 wt. % silver nitrate, 90 wt. % candelilla wax;
15 wt. % silver nitrate, 85 wt. % candelilla wax;
25 wt. % silver nitrate, 75 wt. % candelilla wax;
40 wt. % silver nitrate, 60 wt. % candelilla wax;
5 wt. % silver nitrate, 95 wt. % paraffin wax;
10 wt. % silver nitrate, 90 wt. % paraffin wax;
15 wt. % silver nitrate, 85 wt. % paraffin wax;
25 wt. % silver nitrate, 75 wt. % paraffin wax;
40 wt. % silver nitrate, 60 wt. % paraffin wax;
5 wt. % silver nitrate, 95 wt. % glyceryl tripalmitate;
10 wt. % silver nitrate, 90 wt. % glyceryl tripalmitate;
15 wt. % silver nitrate, 85 wt. % glyceryl tripalmitate;
25 wt. % silver nitrate, 75 wt. % glyceryl tripalmitate;
40 wt. % silver nitrate, 60 wt. % glyceryl tripalmitate;
5 wt. % silver nitrate, 95 wt. % sodium carboxymethylcellulose;
10 wt. % silver nitrate, 90 wt. % sodium carboxymethylcellulose;
15 wt. % silver nitrate, 85 wt. % sodium carboxymethylcellulose;
25 wt. % silver nitrate, 75 wt. % sodium carboxymethylcellulose;
40 wt. % silver nitrate, 60 wt. % sodium carboxymethylcellulose;
5 wt. % silver nitrate, 95 wt. % stearyl alcohol;
10 wt. % silver nitrate, 90 wt. % stearyl alcohol;
15 wt. % silver nitrate, 85 wt. % stearyl alcohol;
25 wt. % silver nitrate, 75 wt. % stearyl alcohol;
40 wt. % silver nitrate, 60 wt. % stearyl alcohol;
5 wt. % silver nitrate, 95 wt. % cetyl alcohol;
10 wt. % silver nitrate, 90 wt. % cetyl alcohol;
15 wt. % silver nitrate, 85 wt. % cetyl alcohol;
25 wt. % silver nitrate, 75 wt. % cetyl alcohol;
40 wt. % silver nitrate, 60 wt. % cetyl alcohol;
5 wt. % silver nitrate, 95 wt. % polyvinyl alcohol;
10 wt. % silver nitrate, 90 wt. % polyvinyl alcohol;
15 wt. % silver nitrate, 85 wt. % polyvinyl alcohol;
25 wt. % silver nitrate, 75 wt. % polyvinyl alcohol;
40 wt. % silver nitrate, 60 wt. % polyvinyl alcohol;
5 wt. % silver nitrate, 95 wt. % 1,2-octanediol;
10 wt. % silver nitrate, 90 wt. % 1,2-octanediol;
15 wt. % silver nitrate, 85 wt. % 1,2-octanediol;
25 wt. % silver nitrate, 75 wt. % 1,2-octanediol;
40 wt. % silver nitrate, 60 wt. % 1,2-octanediol;
10 wt. % SSD, 45 wt. % glyceryl tristearate, 45 wt. % HPMC;
20 wt. % SSD. 40 wt. % glyceryl tristearate, 40 wt. % HPMC;
40 wt. % SSD. 30 wt. % glyceryl tristearate, 30 wt. % HPMC;
15 wt. % silver nitrate, 65 wt. % glyceryl tristearate, 10 wt. % stearyl alcohol;
20 wt. % silver nitrate, 70 wt. % glyceryl tristearate, 10 wt. % stearyl alcohol;
15 wt. % silver nitrate, 65 wt. % glyceryl tripalmitate, 10 wt. % stearyl alcohol;
20 wt. % silver nitrate, 70 wt. % glyceryl tripalmitate, 10 wt. % stearyl alcohol;
15 wt. % silver nitrate, 5 wt. % lidocaine, 80 wt. % candelilla wax;
20 wt. % silver nitrate, 5 wt. % lidocaine, 75 wt. % candelilla wax;
15 wt. % silver nitrate, 5 wt. % lidocaine, 80 wt. % paraffin wax;
20 wt. % silver nitrate, 5 wt. % lidocaine, 75 wt. % paraffin wax;
15 wt. % silver nitrate, 5 wt. % lidocaine, 80 wt. % glyceryl tripalmitate;
20 wt. % silver nitrate, 5 wt. % lidocaine, 75 wt. % glyceryl tripalmitate;
15 wt. % silver nitrate, 5 wt. % lidocaine, 70 wt. % glyceryl tristearate, 10 wt. % stearyl alcohol;
15 wt. % silver nitrate, 5 wt. % lidocaine, 70 wt. % glyceryl tripalmitate, 10 wt. % stearyl alcohol;
15 wt. % silver nitrate, 85 wt. % Softisan® 378 triglyceride blend;
25 wt. % silver nitrate, 75 wt. % Softisan® 378 triglyceride blend;
40 wt. % silver nitrate, 60 wt. % Softisan® 378 triglyceride blend;
10 wt. % silver nitrate, 90 wt. % Witepsol H15;
20 wt. % silver nitrate, 80 wt. % Witepsol H15;
40 wt. % silver nitrate, 60 wt. % Witepsol H15;
15 wt. % fosfomycin, 85 wt. % candelilla wax;
25 wt. % fosfomycin, 75 wt. % candelilla wax;
40 wt. % fosfomycin, 60 wt. % candelilla wax;
15 wt. % fosfomycin, 85 wt. % paraffin wax;
25 wt. % fosfomycin, 75 wt. % paraffin wax;
40 wt. % fosfomycin, 60 wt. % paraffin wax;
15 wt. % fosfomycin, 85 wt. % glyceryl tripalmitate;
25 wt. % fosfomycin, 75 wt. % glyceryl tripalmitate;
40 wt. % fosfomycin, 60 wt. % glyceryl tripalmitate;
15 wt. % fosfomycin, 85 wt. % sodium carboxymethylcellulose;
25 wt. % fosfomycin, 75 wt. % sodium carboxymethylcellulose;
40 wt. % fosfomycin, 60 wt. % sodium carboxymethylcellulose;
15 wt. % fosfomycin, 85 wt. % stearyl alcohol;
25 wt. % fosfomycin, 75 wt. % stearyl alcohol;
40 wt. % fosfomycin, 60 wt. % stearyl alcohol;
15 wt. % fosfomycin, 85 wt. % cetyl alcohol;
25 wt. % fosfomycin, 75 wt. % cetyl alcohol;
40 wt. % fosfomycin, 60 wt. % cetyl alcohol;
15 wt. % fosfomycin, 85 wt. % polyvinyl alcohol;
25 wt. % fosfomycin, 75 wt. % polyvinyl alcohol;
40 wt. % fosfomycin, 60 wt. % polyvinyl alcohol;
15 wt. % fosfomycin, 85 wt. % 1,2-octanediol;
25 wt. % fosfomycin, 75 wt. % 1,2-octanediol;
40 wt. % fosfomycin, 60 wt. % 1,2-octanediol;
15 wt. % fosfomycin, 65 wt. % glyceryl tristearate, 10 wt. % stearyl alcohol;
20 wt. % fosfomycin, 70 wt. % glyceryl tristearate, 10 wt. % stearyl alcohol;
15 wt. % fosfomycin, 65 wt. % glyceryl tripalmitate, 10 wt. % stearyl alcohol;
20 wt. % fosfomycin, 70 wt. % glyceryl tripalmitate, 10 wt. % stearyl alcohol;
15 wt. % fosfomycin, 5 wt. % lidocaine, 80 wt. % candelilla wax;
20 wt. % fosfomycin, 5 wt. % lidocaine, 75 wt. % candelilla wax;
15 wt. % fosfomycin, 5 wt. % lidocaine, 80 wt. % paraffin wax;
20 wt. % fosfomycin, 5 wt. % lidocaine, 75 wt. % paraffin wax;
15 wt. % fosfomycin, 5 wt. % lidocaine, 80 wt. % glyceryl tripalmitate;
20 wt. % fosfomycin, 5 wt. % lidocaine, 75 wt. % glyceryl tripalmitate;
15 wt. % fosfomycin, 5 wt. % lidocaine, 70 wt. % glyceryl tristearate, 10 wt. % stearyl alcohol;
15 wt. % fosfomycin, 5 wt. % lidocaine, 70 wt. % glyceryl tripalmitate, 10 wt. % stearyl alcohol;
15 wt. % fosfomycin, 85 wt. % Softisan® 378 triglyceride blend;
25 wt. % fosfomycin, 75 wt. % Softisan® 378 triglyceride blend;
40 wt. % fosfomycin, 60 wt. % Softisan® 378 triglyceride blend;
10 wt. % fosfomycin, 90 wt. % Witepsol H15;
20 wt. % fosfomycin, 80 wt. % Witepsol H15;
40 wt. % fosfomycin, 60 wt. % Witepsol H15;
15 wt. % lidocaine, 85 wt. % candelilla wax;
25 wt. % lidocaine, 75 wt. % candelilla wax;
40 wt. % lidocaine, 60 wt. % candelilla wax;
15 wt. % lidocaine, 85 wt. % paraffin wax;
25 wt. % lidocaine, 75 wt. % paraffin wax;
40 wt. % lidocaine, 60 wt. % paraffin wax;
15 wt. % lidocaine, 85 wt. % glyceryl tripalmitate;
25 wt. % lidocaine, 75 wt. % glyceryl tripalmitate;
40 wt. % lidocaine, 60 wt. % glyceryl tripalmitate;
15 wt. % lidocaine, 85 wt. % sodium carboxymethylcellulose;
25 wt. % lidocaine, 75 wt. % sodium carboxymethylcellulose;
40 wt. % lidocaine, 60 wt. % sodium carboxymethylcellulose;
15 wt. % lidocaine, 85 wt. % stearyl alcohol;
25 wt. % lidocaine, 75 wt. % stearyl alcohol;
40 wt. % lidocaine, 60 wt. % stearyl alcohol;
15 wt. % lidocaine, 85 wt. % cetyl alcohol;
25 wt. % lidocaine, 75 wt. % cetyl alcohol;
40 wt. % lidocaine, 60 wt. % cetyl alcohol;
15 wt. % lidocaine, 85 wt. % polyvinyl alcohol;
25 wt. % lidocaine, 75 wt. % polyvinyl alcohol;
40 wt. % lidocaine, 60 wt. % polyvinyl alcohol;
15 wt. % lidocaine, 85 wt. % 1,2-octanediol;
25 wt. % lidocaine, 75 wt. % 1,2-octanediol;
40 wt. % lidocaine, 60 wt. % 1,2-octanediol;
15 wt. % lidocaine, 65 wt. % glyceryl tristearate, 10 wt. % stearyl alcohol;
20 wt. % lidocaine, 70 wt. % glyceryl tristearate, 10 wt. % stearyl alcohol;
15 wt. % lidocaine, 65 wt. % glyceryl tripalmitate, 10 wt. % stearyl alcohol;
20 wt. % lidocaine, 70 wt. % glyceryl tripalmitate, 10 wt. % stearyl alcohol;
15 wt. % lidocaine, 85 wt. % Softisan® 378 triglyceride blend;
25 wt. % lidocaine, 75 wt. % Softisan® 378 triglyceride blend;
40 wt. % lidocaine, 60 wt. % Softisan® 378 triglyceride blend;
10 wt. % lidocaine, 90 wt. % Witepsol H15;
20 wt. % lidocaine, 80 wt. % Witepsol H15;
40 wt. % lidocaine, 60 wt. % Witepsol H15;
15 wt. % mitomycin, 85 wt. % candelilla wax;
25 wt. % mitomycin, 75 wt. % candelilla wax;
40 wt. % mitomycin, 60 wt. % candelilla wax;
15 wt. % mitomycin, 85 wt. % paraffin wax;
25 wt. % mitomycin, 75 wt. % paraffin wax;
40 wt. % mitomycin, 60 wt. % paraffin wax;
15 wt. % mitomycin, 85 wt. % glyceryl tripalmitate;
25 wt. % mitomycin, 75 wt. % glyceryl tripalmitate;
40 wt. % mitomycin, 60 wt. % glyceryl tripalmitate;
15 wt. % mitomycin, 85 wt. % sodium carboxymethylcellulose;
25 wt. % mitomycin, 75 wt. % sodium carboxymethylcellulose;
40 wt. % mitomycin, 60 wt. % sodium carboxymethylcellulose;
15 wt. % mitomycin, 85 wt. % stearyl alcohol;
25 wt. % mitomycin, 75 wt. % stearyl alcohol;
40 wt. % mitomycin, 60 wt. % stearyl alcohol;
15 wt. % mitomycin, 85 wt. % cetyl alcohol;
25 wt. % mitomycin, 75 wt. % cetyl alcohol;
40 wt. % mitomycin, 60 wt. % cetyl alcohol;
15 wt. % mitomycin, 85 wt. % polyvinyl alcohol;
25 wt. % mitomycin, 75 wt. % polyvinyl alcohol;
40 wt. % mitomycin, 60 wt. % polyvinyl alcohol;
15 wt. % mitomycin, 85 wt. % 1,2-octanediol;
25 wt. % mitomycin, 75 wt. % 1,2-octanediol;
40 wt. % mitomycin, 60 wt. % 1,2-octanediol;
15 wt. % mitomycin, 65 wt. % glyceryl tristearate, 10 wt. % stearyl alcohol;
20 wt. % mitomycin, 70 wt. % glyceryl tristearate, 10 wt. % stearyl alcohol;
15 wt. % mitomycin, 65 wt. % glyceryl tripalmitate, 10 wt. % stearyl alcohol;
20 wt. % mitomycin, 70 wt. % glyceryl tripalmitate, 10 wt. % stearyl alcohol;
15 wt. % mitomycin, 5 wt. % lidocaine, 80 wt. % candelilla wax;
20 wt. % mitomycin, 5 wt. % lidocaine, 75 wt. % candelilla wax;
15 wt. % mitomycin, 5 wt. % lidocaine, 80 wt. % paraffin wax;
20 wt. % mitomycin, 5 wt. % lidocaine, 75 wt. % paraffin wax;
15 wt. % mitomycin, 5 wt. % lidocaine, 80 wt. % glyceryl tripalmitate;
20 wt. % mitomycin, 5 wt. % lidocaine, 75 wt. % glyceryl tripalmitate;
15 wt. % mitomycin, 5 wt. % lidocaine, 70 wt. % glyceryl tristearate, 10 wt. % stearyl alcohol;
15 wt. % mitomycin, 5 wt. % lidocaine, 70 wt. % glyceryl tripalmitate, 10 wt. % stearyl alcohol;
15 wt. % mitomycin, 85 wt. % Softisan® 378 triglyceride blend;
25 wt. % mitomycin, 75 wt. % Softisan® 378 triglyceride blend;
40 wt. % mitomycin, 60 wt. % Softisan® 378 triglyceride blend;
10 wt. % mitomycin, 90 wt. % Witepsol H15;
20 wt. % mitomycin, 80 wt. % Witepsol H15;
40 wt. % mitomycin, 60 wt. % Witepsol H15;
15 wt. % gemcitabine, 85 wt. % candelilla wax;
25 wt. % gemcitabine, 75 wt. % candelilla wax;
40 wt. % gemcitabine, 60 wt. % candelilla wax;
15 wt. % gemcitabine, 85 wt. % paraffin wax;
25 wt. % gemcitabine, 75 wt. % paraffin wax;
40 wt. % gemcitabine, 60 wt. % paraffin wax;
15 wt. % gemcitabine, 85 wt. % glyceryl tripalmitate;
25 wt. % gemcitabine, 75 wt. % glyceryl tripalmitate;
40 wt. % gemcitabine, 60 wt. % glyceryl tripalmitate;
15 wt. % gemcitabine, 85 wt. % sodium carboxymethylcellulose;
25 wt. % gemcitabine, 75 wt. % sodium carboxymethylcellulose;
40 wt. % gemcitabine, 60 wt. % sodium carboxymethylcellulose;
15 wt. % gemcitabine, 85 wt. % stearyl alcohol;
25 wt. % gemcitabine, 75 wt. % stearyl alcohol;
40 wt. % gemcitabine, 60 wt. % stearyl alcohol;
15 wt. % gemcitabine, 85 wt. % cetyl alcohol;
25 wt. % gemcitabine, 75 wt. % cetyl alcohol;
40 wt. % gemcitabine, 60 wt. % cetyl alcohol;
15 wt. % gemcitabine, 85 wt. % polyvinyl alcohol;
25 wt. % gemcitabine, 75 wt. % polyvinyl alcohol;
40 wt. % gemcitabine, 60 wt. % polyvinyl alcohol;
15 wt. % gemcitabine, 85 wt. % 1,2-octanediol;
25 wt. % gemcitabine, 75 wt. % 1,2-octanediol;
40 wt. % gemcitabine, 60 wt. % 1,2-octanediol;
15 wt. % gemcitabine, 65 wt. % glyceryl tristearate, 10 wt. % stearyl alcohol;
20 wt. % gemcitabine, 70 wt. % glyceryl tristearate, 10 wt. % stearyl alcohol;
15 wt. % gemcitabine, 65 wt. % glyceryl tripalmitate, 10 wt. % stearyl alcohol;
20 wt. % gemcitabine, 70 wt. % glyceryl tripalmitate, 10 wt. % stearyl alcohol;
15 wt. % gemcitabine, 5 wt. % lidocaine, 80 wt. % candelilla wax;
20 wt. % gemcitabine, 5 wt. % lidocaine, 75 wt. % candelilla wax;
15 wt. % gemcitabine, 5 wt. % lidocaine, 80 wt. % paraffin wax;
20 wt. % gemcitabine, 5 wt. % lidocaine, 75 wt. % paraffin wax;
15 wt. % gemcitabine, 5 wt. % lidocaine, 80 wt. % glyceryl tripalmitate;
20 wt. % gemcitabine, 5 wt. % lidocaine, 75 wt. % glyceryl tripalmitate;
15 wt. % gemcitabine, 5 wt. % lidocaine, 70 wt. % glyceryl tristearate, 10 wt. % stearyl alcohol;
15 wt. % gemcitabine, 5 wt. % lidocaine, 70 wt. % glyceryl tripalmitate, 10 wt. % stearyl alcohol;
15 wt. % gemcitabine, 85 wt. % Softisan® 378 triglyceride blend;
25 wt. % gemcitabine, 75 wt. % Softisan® 378 triglyceride blend;
40 wt. % gemcitabine, 60 wt. % Softisan® 378 triglyceride blend;
10 wt. % gemcitabine, 90 wt. % Witepsol H15;
20 wt. % gemcitabine, 80 wt. % Witepsol H15; and
40 wt. % gemcitabine, 60 wt. % Witepsol H15.

It will be appreciated from above and elsewhere in this application that the intravesicular formulation can include additional components, including additional particle types (e.g., with different active agents, loading %, and/or different controlled release carriers), a liquid carrier in which the particles are dispersed, and excipients such as pH-adjusting agents, viscosity adjusting agents, dispersants, tonicity adjusting agents, preservatives and stabilizers, solubilizers, and emulsifiers.

The target concentration of pharmacologically active agent in the bladder is generally in the range of 10 ppm to 150 ppm, i.e., 10 mg/L to 150 mg/L, in urine, e.g., 10 ppm, 16 ppm, 20 ppm, 35 ppm, 45 ppm, 50 ppm, 75 ppm. 100 ppm, 115 ppm, 125 ppm, and 128 ppm (corresponding to 10 mg/L, 16 mg/L, 20 mg/L, 35 mg/L, 45 mg/L. 50 mg/L, 75 mg/L, 100 mg/L, 115 mg/L, 125 mg/L, and 128 mg/L, respectively), etc., but will, of course, depend on multiple factors, including the active agent administered, the indication, the age, weight, and condition of the subject, and the like. A preferred sub-range is 15 ppm to 130 ppm. Dosage may be calculated by extrapolating from the following example. For a drug delivery period of 90 days, about 100 L will pass through the bladder. Assuming that 100% of the active agent in the particles will be released into the bladder during the 90-day period, achieving a 20 mg/L concentration requires that 2000 mg (2 g) active agent, or 2 g, be delivered to treat the 100 L of urine. For a population of particles that comprises 25% active agent and 75% controlled release carrier, 8 g of particles would have to be administered in order to deliver 2 g of the active agent into the bladder. As another example, during a drug delivery period of 180 days, about 200 L of urine will pass through the bladder. Again, assuming that 100% of the active agent in the particles is released into the bladder during the 180-day period, achieving a 50 mg/L concentration requires that 10,000 mg (10 g) be delivered to treat the 200 L of urine. For a population of particles that, for purposes of illustration, again comprises 25 wt. % active agent and 75 wt. % controlled release carrier, 40 g of particles would have to be administered to deliver 10 g of the active agent to treat the 200 L of urine. The calculation can be readily adjusted for different active agent-to-carrier ratios, drug delivery time periods, and target concentration of active agent in the bladder. It should also be noted that as urine production varies throughout the day, the concentration of active agent within the bladder varies throughout the day as well (and may peak during a time of low urine production).

The extended drug delivery time period during which the active agent is released from the particles into the bladder and, preferably, during which therapeutically effective concentrations of the active agent or a metabolite thereof are provided in the bladder, is in the range of about one month to at least about three months, e.g., about two months to about four months, about three months, at least about three months, four months, five months, six months, etc.

### G. Indications and Administration:

The particle formulations of the invention can be administered to a subject to treat a disorder of the urinary system. The disorder may be a disease or other adverse condition of the bladder, kidneys, ureters, and/or urethra, and is not limited in any respect except that the disorder is responsive or predicted to be responsive to the intravesicular administration of a particular pharmacologically active agent or type of pharmacologically active agent. Examples of urinary system disorders that can be treated according to the invention include urinary system infections (commonly referred to as “UTIs” or urinary tract infections”) such as bacterial, fungal, and viral infections; cancers or benign tumors of the urinary system; urinary incontinence (including urge urinary incontinence, or overactive bladder, “OAB”) and urinary retention; urinary system inflammation, injury, or scarring; and kidney stones, diabetic nephropathy, or kidney failure.

Examples of specific indications and representative active agents to treat the indications are as follows:
Cystitis (an infection of the lower urinary tract), including acute cystitis, chronic cystitis, hemorrhagic cystitis, bacterial cystitis, and emphysematous cystitis, any of which can be caused by a bacterial, fungal, or viral infection and are treated accordingly, with an appropriate anti-infective agent (see Section 2.A for representative antibacterial, anti-fungal, and antiviral agents);
Interstitial cystitis (also known as bladder pain syndrome. BPS), commonly treated with a serotonin reuptake inhibitor (SRI) such as a tricyclic antidepressant (amitriptyline, imipramine, desipramine, etc.), a serotonin-norepinephrine reuptake inhibitor (such as duloxetine or venlafaxine), an anti-inflammatory agent (as described in Section 2.A; ibuprofen is typical), or pentosan polysulfate;
Neurogenic bladder dysfunction, commonly treated with an anti-cholinergic agent (e.g., oxybutynin or tolterodine) to reduce bladder contractions, or with a muscarinic agent (e.g., bethanechol or an a-blocker such as phentolamine) to treat overflow incontinence;
Overactive bladder (OAB; also referred to as “urge urinary incontinence”) syndrome, commonly treated with an antimuscarinic drug (e.g., darifenacin, hyoscyamine, oxybutynin, tolterodine, solifenacin, trospium, or fesoterodine), or with a b3-adrenergic receptor agonist (such as mirabegron or vibegron);
Incontinence caused by OAB, typically treated with an anesthetic agent or an analgesic agent such as those identified in Section 2.A;
Stress incontinence, also known as stress urinary incontinence (SUI), resulting from inadequate closure of the bladder outlet by the urethral sphincter,
Chlamydia (Chlamydia trachomatis) and gonorrhea (Neisseria gonorrhoeae), commonly treated with an antibiotic such as azithromycin, doxycycline, erythromycin, levofloxacin, ofloxacin, or tetracycline (see Section 2.A for additional representative active agents for treating chlamydia);
Gonococcal urethritis, caused by an N. gonorrhoeae infection, typically treated with ceftriaxone or ceftriaxone, in combination with azithromycin;
Non-gonococcal urethritis, usually caused by Chlamydia trachomatis, Trichomonas vaginalis, adenoviridae, uropathogenic E. coli, HSV1 or HSV-2, cytomegalovirus, Ureaplasma urealyticum, methicillin-resistant S. aureas, or Group B streptococcus, most commonly caused by chlamydia, is typically treated with an antibiotic such as azithromycin, doxycycline, erythromycin, levofloxacin, or ofloxacin;
Syphilis, commonly treated with benzathine benzylpenicillin or ceftriaxone;
Pyelonephritis, commonly treated with a fluoroquinolone antibiotic (e.g., ciprofloxacin or levofloxacin), a cephalosporin antibiotic (e.g., ceftriaxone), an aminoglycoside antibiotic (e.g., gentamycin), or fosfomycin;
Kidney stones, which can be treated with, for instance, allopurinol, alkalizing agents, or thiazide diuretics, as explained in Section 2.A; Pain associated with kidney stones, typically treated with an NSAID anti-inflammatory/analgesic or with an opioid analgesic;
Diabetic nephropathy (diabetic kidney disease), treated with an inhibitor of the renin-angiotensin-aldosterone system (RAAS) such as an angiotensin converting enzyme (ACE) inhibitor, an angiotensin receptor blocker, a direct renin inhibitor, or a mineralocorticoid antagonist, or with an anti-diabetic agent (see Section 2.A);
Vesicoureteral reflux (VUR), a disorder that increases the likelihood of a urinary system infection, particularly in children; and
Cancers or benign tumors of the urinary system, treated with a chemotherapeutic agent as set forth in Section 2.A, particularly apaziquone, fosfomycin, gemcitabine, and BCG immunotherapy.

### Administration

The intravesicular formulation is administered using any known or hereinafter developed device suitable for introducing a pharmaceutical formulation into bladder. For instance, the formulation may be administered through a transurethral syringe, a Toomey syringe, or via a catheter.

Intravesicular administration of a formulation of the invention is particularly useful in the treatment of individuals for whom a therapeutically effective dose of an oral medication is likely to be problematic, including the elderly, who make up the majority of individuals afflicted with recurring and/or serious urinary system disorders, and children. The invention additionally finds utility in the treatment of individuals afflicted with any of a wide range of conditions and diseases that increase the likelihood of a urinary system disorder, particularly complicated urinary tract infections. Such individuals include subjects with lupus and other systemic autoimmune diseases; subjects on immunosuppressive therapy; subjects with a neurological disorder; subjects with a sexually transmitted disease; diabetic patients; cancer patients; patients with a functional or anatomic abnormality of the urinary tract; patients who experienced a UTI as a child; patients who have had extensive antimicrobial therapy; patients who have contracted a nosocomial infection; and numerous others.

### Other Uses

While the formulation/particles are described for use in treating ailments of the urinary tract (e.g., UTI), the skilled artisan will appreciate other uses of the invention. For example, the particles can be coated with an agent such as tissue plasminogen activator or streptokinase. They can be instilled into an anatomic space such as the pleural cavity of the chest for the purpose of lysing adhesions and loculations from sterile or infected hydrothorax. The particles can be inserted surgically or through a chest thoracostomy tube. The particles will float to the top of the cavity, selectively delivering a therapy to break the abscess/loculation and connect it to an adjacent space, therapy more effectively draining the thoracic cavity and allowing better expansion of the lung into the thorax.

The platform can be used with a drug whose target is preferentially in the apical portion of an anatomic space, potential space, surgical space, or pathological space would benefit from a buoyant drug deliver particle. For example, in apical blebs causing pneumothorax, there is a desire to have targeted irritant delivered to the apex of the bladder to cause scarring of the apex to the pleural wall. Loaded particles can preferentially be delivered to this target in the following manner:
a) Dosed rods are inserted into the chest cavity;
b) The chest cavity receives an instillation of fluid, allowing the particles to make use of buoyancy to travel to apex of the space;
c) The fluid is then rapidly drained from the space, leaving the buoyant particles at their target to deliver a high and focused drug concentration or mechanical irritation.

In a similar manner, the particles can be injected through a peritoneal dialysis catheter to break up adhesions and loculations which make the catheter nonfunctional, saving the patient a repeat procedure.

The particles can be inserted through a catheter into an abscess cavity that is in a sensitive area making surgical removal non-ideal (such as face, breast, genital tissue, CNS). The particles can be used in other fluid-filled areas:
a) CSF (spine or skull)
b) Venous sinuses of skull (valveless)
c) Pleural space
d) Peritoneal space
e) Pericardial space

Buoyancy of particles can be used for preferential targeting of therapy into the vascular tree. Specifically, if a main artery has multiple terminal vessels there is no way to deliver targeted therapy but for blanket covering of entire downstream area (shotgun approach) or selective catheter delivery (technically difficult). Buoyancy allows the patient to be positioned such that the particles will preferentially enter the most non-dependent vascular branch (or specifically avoid the most dependent branch).

### EXAMPLES

The following non-limiting examples are provided for illustrative purposes only in order to facilitate a more complete understanding of representative embodiments now contemplated. These examples are intended to be a mere subset of all possible contexts in which the components of the formulation may be combined. Thus, these examples should not be construed to limit any of the embodiments described in the present specification, including those pertaining to the type and amounts of components of the formulation and/or methods and uses thereof.

### Example 1

### Antimicrobial Efficacy of SSD in Artificial Urine

The antimicrobial efficacy of SSD was evaluated in artificial urine against various test microbes by determining the planktonic log reductions after specific contact times. The testing was designed to measure % kill and log reduction of planktonic cultures of several microbes after six different exposure times. The SSD was tested at four different dilutions to demonstrate the kill rate of the silver ions.

Challenge time, dilution range for silver ions, and dilution range for SSD are provided in Table 1:

TABLE 1

| | Challenge time | Dilutions | Dilutions |
|---|---|---|---|
| Sample | (hr) | (Ag⁺), mg/L | (SSD), mg/L |
| SSD | 0, 1, 2, 4, 6 | 3, 5, 10, 20 | 9.93, 16.55, 33.11, 66.22 |
| Negative | 0, 1, 2, 4, 6 | — | — |
| control | | | |

Artificial urine was prepared as described by Brooks et al. (1997), “A simple artificial urine for growth of urinary pathogens,” Lett Appl Microbiol 24 (3): 203-6, sterilized by filtration through 0.2 mm filter units, and stored at 4° C. Microorganisms, growth media, and conditions are set forth in Table 2:

TABLE 2

| Organism | Growth Media and Conditions* |
|---|---|
| Escherichia coli | Tryptic soy agar (TSA), humidified incubator at |
| ATCC 700928 | 37 ± 2° C. for 24 hr |
| Klebsiella | TSA, humidified incubator at 37 ± 2° C. for 24 hr |
| pneumonia | |
| ATCC BAA-1705 | |
| Staphylococcus | TSA, humidified incubator at 37 ± 2° C. for 24 hr |
| saprophyticus | |
| ATCC 15305 | |
| Proteus mirabilis | TSA, humidified incubator at 37 ± 2° C. for 24 hr |
| ATCC 29906 | |
| Enterococcus | TSA, humidified incubator at 37 ± 2° C. for 24 hr |
| faecalis | |
| ATCC 29212 | |
| Staphylococcus | TSA, humidified incubator at 37 ± 2° C. for 24 hr |
| aureas | |
| ATCC 6538 | |
| Candida albicans | TSA, Sabouraud dextrose broth and agar (SDB/SDA), |
| ATCC 18804 | humidified incubator at 37 ± 2° C. for 24 hr |
| Streptococcus | TSA, humidified incubator at 37 ± 2° C. for 24 hr |
| agalactiae | |
| GP #7 | |

The organisms in Table 2 were tested using exposure time points of 1, 2, 4, and 6 (as indicated in Table 1) from the same plates over time. Three plates were needed per strain, for a total of 27 challenge plates. Three replicate samples were used, with sterility controls and growth controls used for each experiment. Working solutions were made up of either the test or control compositions according to Table 1, and 4.8 mL of the working solutions were added to the appropriate wells of the challenge plates.

### Inoculum Preparation

Using a cryogenic stock (at −70° C.), the first sub-culture of the microorganisms was struck out on appropriate media (TSA plates for bacterial isolates or SDA plates for C. albicans, as indicated in Table 2).

Plates were incubated at 37±2° C. for 24 hours and stored at 4±1° C. until needed. From the first sub-cultures, second sub-cultures were struck out on appropriate media (again, TSA for the bacterial isolates or SDB/SDA for C. albicans). From the first sub-cultures, second sub-cultures were struck out on appropriate media (TSA for the bacterial isolates or SDB/SDA for C. albicans). Approximately 4-5 large, or 5-10 small, well-isolated colonies from the second subculture plate were emulsified in distilled, deionized water and adjusted to achieve a turbidity equivalent to a 0.5 McFarland standard.

Bacteria were emulsified in 6 mL water, while C. albicans was emulsified in 12 ml distilled water. Cell suspensions were centrifuged (3000×g for 10 min), and the pellets were washed 2× by decanting the supernatant and resuspending the cell pellet in equal volumes of distilled deionized water. Following the washing, the cell pellets were resuspended in distilled deionized water Bacteria were resuspended in 20 ml water, while C. albicans was resuspended in 4 ml distilled water.

The cell density of the inoculum was confirmed by serially diluting and spot plating. A purity check was performed by checking the spot plates for the presence of abnormal looking colonies after 16-20 hours of incubation. All strains were determined to be pure cultures.

### Inoculation of Challenge Plates

200 μL of the inoculum was added to each well of the challenge plates, except the sterility control (SC) wells. 200 μL of sterile water was added to the SC wells. Inoculated plates were incubated at 37±2° C. in a non-CO₂ incubator and samples were taken at 0, 1, 2, 4 and 6 hour timepoints.

### Recovery

Following each of the challenge time points, 100 μL was mixed with 100 μL modified D/E neutralizer composition (D/E neutralizing broth, 5 g/L L-cysteine, and 5 g/L glutathione, 2× strength) in the most concentrated of the serial dilution plates. 180 μL of sterile 0.9% saline was placed in the remaining dilutions. Serial dilutions of 1 to 107 were prepared. 10 μL from each well was then removed and spot plated onto prepared TSA or SDA plates. Plates were incubated at 37±2° C. and the number of resulting colonies were counted after approximately 16-24 hours of incubation. Data was evaluated as log 10 CFU/mL.

### Neutralization Validation

200 μL of each inoculum was placed into triplicate wells, per strain, containing 2 mL of the modified D/E neutralizer composition, and 1.8 mL of the 20 ppm Ag+ test solution. After 5 min, 100 μL from each well of the neutralization efficacy plates was pipetted into the first row of a 96-well microtiter plate. 180 μL of sterile 0.9% saline was placed in the remaining rows. Serial dilutions were prepared, 10 μL from each well was removed and spot plated, plates were incubated, and data evaluated as described under “Recovery.”

### Calculations and Statistics

$$Spot C F U / mL = ( C F U / 10 µL ) / 0.01 \\ Log 10 ( C F U / mL ) = Log 10 ( C F U / mL + 1 ) \\ Log 10 reduction = Log 10 ( C F U / mL ) ( growth control ) - Log 10 ( C F U / mL ) ( test conditions ) \\ \% Kill = ( ( antilog avg ( growth control ) - antilog avg ( test conditions ) ) / antilog avg ( growth control ) ) \times 100$$

P-values compared to the growth control were a one-way ANOVA with a Tukey multiple comparisons Post Test.

### Results

A Log₁₀ reduction>4 was observed for E. coli, K. pneumoniae, P. mirabilis and C. albicans at all four concentrations of SSD tested, after 4 hours. A Log₁₀ reduction>3 was observed for S. saprophyticus, after 6 hours, at all four concentrations of SSD tested. There was no detectable difference in the amount, or rate, of killing between the four different concentrations of SSD tested.

### Example 2

### Antimicrobial Efficacy of SSD and Cefpodoxime Against UTI Bacteria Isolates

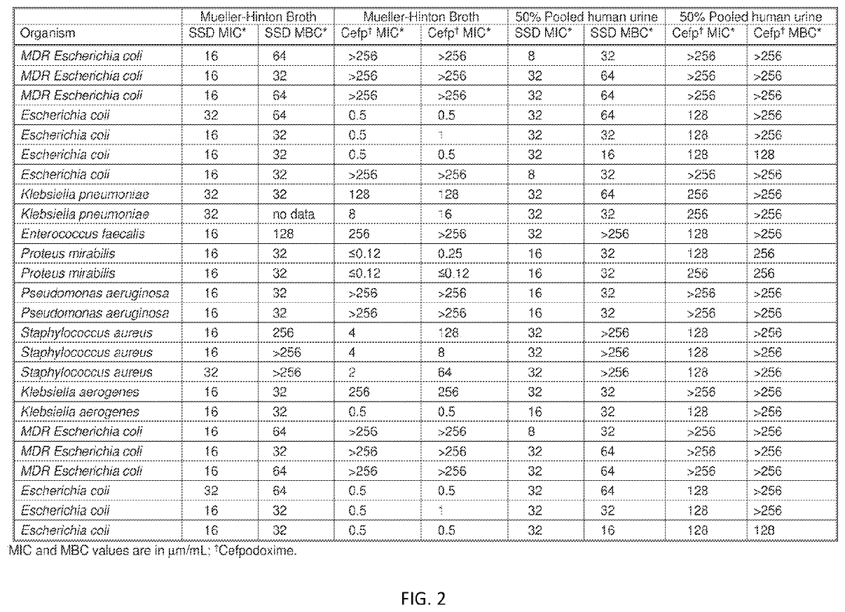

The antimicrobial efficacy of SSD and cefpodoxime was evaluated with respect to the bactericidal effect of SSD against the organisms listed in Table 2 (detailed in FIG. 2) in addition to two E. coli multi-drug resistant (MDR) clinical isolates. Cefpodoxime, a commonly prescribed antibiotic, was used as a comparator agent. The minimum inhibitory concentration (MIC) and minimum bactericidal concentration (MBC) were identified. These define a drug's potency in terms of the concentration at which it will inhibit growth of (MIC) or completely kill (MBC) 1×10⁶ challenge microorganisms during an 18 hr to 20 hr period of incubated (35±2° C.) exposure. MIC values for SSD and cefpodoxime against each of the 10 isolates were generated using reference frozen-form broth microdilution susceptibility panels and 50% pooled human urine in accordance with Clinical and Laboratory Standards Institute (CLSI) guidelines. Both SSD and cefpodoxime were tested over 15 doubling dilutions (1:2 serial dilutions) and bacterial colony counts were done on each isolate tested to determine the starting inoculum concentrations (target 5×105 CFU/mL). The results are shown in FIG. 2 (Table 3).

### Example 3

### Particle Fabrication:

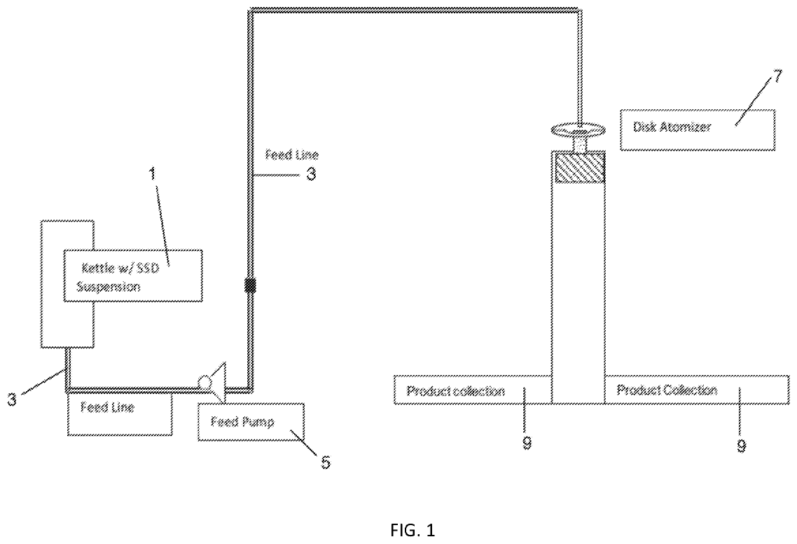

A Melt Spray Congealing (MSC) process was used to prepared particles with glyceryl tristearate (Dynasan® 118, from IOI Oleochemicals GmbH, hereinafter “Dynasan”) as the controlled release carrier and silver sulfadiazine (SSD), an antibiotic, as the pharmacologically active agent. The process involves dispersing SSD in a Dynasan melt and then atomizing the composition so provided using a rotary atomizer. The atomized droplets are allowed to congeal resulting in SSD being entrapped in the Dynasan matrix. The MSC fabrication process is illustrated schematically in FIG. 1. The particle size of the MSC particles can be adjusted by controlling the feed material viscosity, rotational speed, and feed rate, as well as disk design and disk size. Decreasing feed material viscosity, rotational speed, and/or feed rate will provide larger particles.

MSC protocol used to prepare particles with 25% silver sulfadiazine and 75% Dynasan: Referring to FIG. 1, the Dynasan (40 g) was melted in a kettle 1 at 90° C. After melting was complete, SSD (13.3 g) was added to the kettle containing the melted Dynasan and dispersed therein using homogenization up to 20,000 rpm for approximately 3 minutes. The SSD suspension so prepared was then fed at a rate of 75 g/min to 100 g/min) into feed line 3 and through feed pump 5 onto a 4″-disk rotary atomizer 7 heated to approximately 90° C. to 100° C. and rotating at approximately 2400 rpm. The SSD suspension formed a thin film across the disk surface and was atomized into distinct droplets at its periphery. The atomized droplets congealed in flight and were collected (40 g, 75% yield) on a tray 9, but can be collected in a plastic enclosure, or by use of a cyclone separator, or using other suitable means. To ensure a narrow particle size distribution, a sieve can be used to sift out particles outside of the desired size range.

### Example 4

### Particle Density Evaluations:

Controlled release particles were prepared with SSD and a controlled release carrier (CRC) at different ratios, and particle density was evaluated. Density of various materials are provided in Table 4, while CRCs, SSD:CRC weight ratios in the particles, CRC wt. %, CCR density, and (theoretical) particle density are set forth in Table 5:

TABLE 4

| Material | Density (g/cm³) |
|---|---|
| SSD | 1.496 |
| Glyceryl trimyristate | 0.885 |
| Glyceryl tripalmitate | 0.8752 |
| Glyceryl tristearate | 0.862 |
| Stearyl alcohol | 0.812 |
| Glyceryl behenate | 0.942 |
| Poloxamer P407 | 1.05 |
| Urine | 1.027 |
| 0.9% saline | 1.0046 |

TABLE 5

| | | | | CCR | particle |
|---|---|---|---|---|---|
| | | SSD:CCR | CCR | Density | density |
| # | CCR | (wt/wt) | wt. % | (g/cm³) | (g/cm³) |
| 1 | Glyceryl trimyristate | 10:90 | 90 | 0.885 | 21.0965 |
| 2 | Glyceryl trimyristate | 25:75 | 75 | 0.885 | 51.41375 |
| 3 | Glyceryl trimyristate | 40:60 | 60 | 0.885 | 81.731 |
| 4 | Glyceryl tripalmitate | 10:90 | 90 | 0.8752 | 21.08768 |
| 5 | Glyceryl tripalmitate | 25:75 | 75 | 0.8752 | 51.4064 |
| 6 | Glyceryl tripalmitate | 40:60 | 60 | 0.8752 | 81.72512 |
| 7 | Glyceryl tristearate | 10:90 | 90 | 0.862 | 21.0758 |
| 8 | Glyceryl tristearate | 25:75 | 75 | 0.862 | 51.3965 |
| 9 | Glyceryl tristearate | 40:60 | 60 | 0.862 | 81.7172 |
| 10 | Stearyl alcohol | 10:90 | 90 | 0.812 | 21.0308 |
| 11 | Stearyl alcohol | 25:75 | 75 | 0.812 | 51.359 |
| 12 | Stearyl alcohol | 40:60 | 60 | 0.812 | 81.6872 |
| 13 | Glyceryl behenate | 10:90 | 90 | 0.942 | 21.1478 |
| 14 | Glyceryl behenate | 25:75 | 75 | 0.942 | 51.4565 |
| 15 | Glyceryl behenate | 40:60 | 60 | 0.942 | 81.7652 |
| 16 | Poloxamer P407 | 10:90 | 90 | 1.05 | 21.245 |
| 17 | Poloxamer P407 | 25:75 | 75 | 1.05 | 51.5375 |
| 18 | Poloxamer P407 | 40:60 | 60 | 1.05 | 81.83 |

### Example 5

### SSD Particle Release Profile:

particles containing 25 wt. % SSD and 75 wt. % Dynasan® 118 were prepared as described in Example 3, and dispersed in PBS buffered to a pH of 7.4. The concentration of particles in PBS was 10 mg/mL, with a corresponding concentration of 2.5 mg/mL for active agent in the formulation. The mean particle diameter (i.e., d(0.5)) was found to be 174 mm.

The amount of drug released over time was evaluated using a dissolution test conducted at 37° C. with constant stirring. The results are presented in Table 6, which shows the amount of SSD released, the percentage of total SSD released, and the rate of SSD release (% SSD released per hr) at different time points during the evaluation:

TABLE 6

| Time (hr) | SSD mg released | % SSD released | % SSD released/hr |
|---|---|---|---|
| 3 | 0.027 | 1.175 | 0.392 |
| 5 | 0.038 | 1.654 | 0.631 |
| 23 | 0.051 | 2.202 | 0.662 |
| 96 | 0.092 | 3.962 | 0.686 |
| 120 | 0.117 | 5.059 | 0.732 |
| 143 | 0.139 | 6.010 | 0.773 |

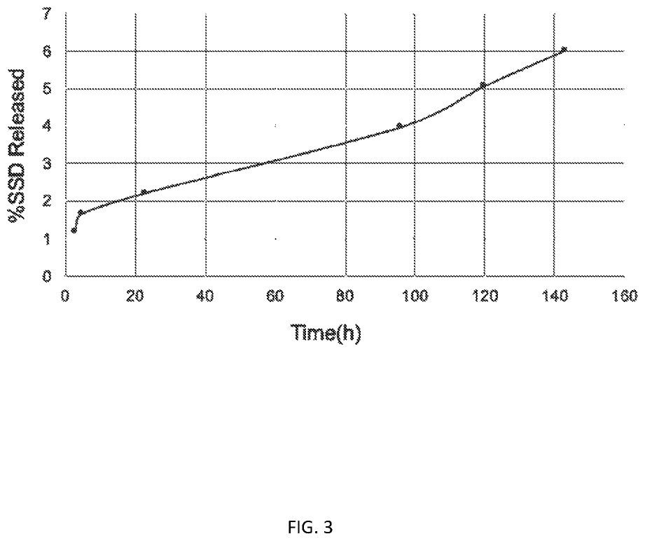

The percentage of SSD released over time was plotted. The results, shown in FIG. 3, illustrate that zero order release kinetics were achieved, i.e., the SSD was released from the particles at an approximately constant rate. At the release rate calculated, the particle formulation would provide controlled release drug delivery in the bladder for up to three months.

### Example 6

### SSD Particle Release Profile:

Two SSD particle formulations were prepared, both with 20% SSD loading and 75% GTS, with one formulation prepared in phosphate-buffered saline (PBS) and the other in artificial urine media (AUM). Particle size was 545 μm, the pH of the formulations was 7.5, and the concentration of particles in the liquid carrier, in both formulations, was 50 mg/mL (with a corresponding SSD concentration of 10 mg/mL).

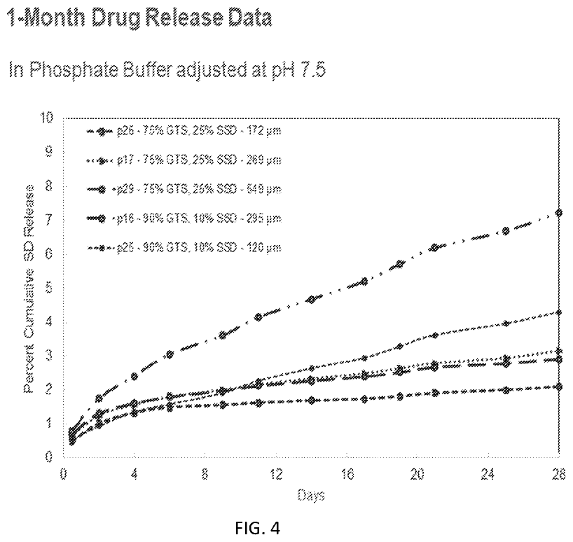

The amount of drug released over time was evaluated using a USP Dissolution Apparatus I conducted at 37° C. with constant stirring. 90% of diluent was removed and replaced at every sampling interval in order to mimic bladder function. The results are presented in in FIGS. 4 and 5.

### Example 7

### Evaluating Particle Degradation:

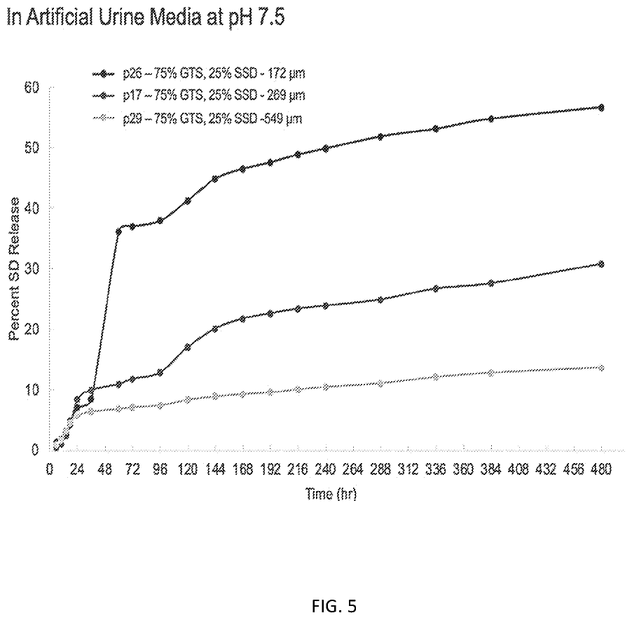

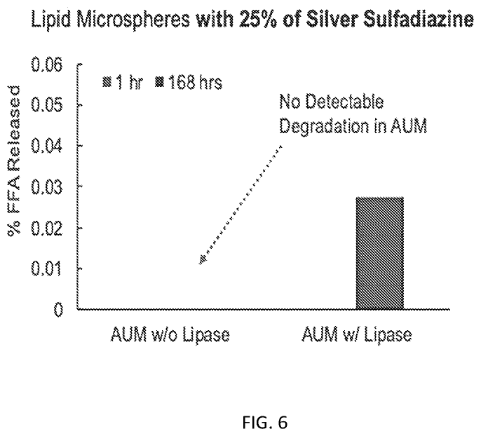

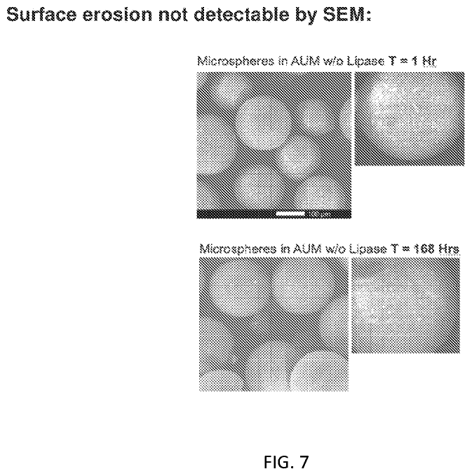

FIG. 6 and FIG. 7 show results from a degradation assay to evaluate degradation of glyceryl tristearate (GTS)/silver sulfadiazine (SSD) particles in artificial urine medium (AUM). The assay detects the presence or absence of stearic acid resulting from hydrolytic cleavage of the GTS carrier over time. GTS/SSD microspheres were submerged in AUM with and without lipase for 7 days (168 hrs), incubated at 37° C. and mixed at 100 rpm. As lipase is an esterase, catalyzing hydrolytic cleavage of esters, glyceryl tristearate is hydrolyzed by lipase to yield glycerol and stearic acid; lipase is not present in urine or in the AUM. Accordingly, the group of microspheres that was immersed in AUM with lipase acted as the positive control group. After 7 days, media was collected from both groups and an ELISA assay that detects long-chain free fatty acid (FFA) groups was run (abcam, “Free Fatty Acid Assay Kit—Quantification,” ab65341). In the FFA assay protocol, fatty acids are converted to their CoA derivatives (coenzyme A), which are subsequently oxidized, leading to formation of color/fluorescence. Fatty acids were then quantified by either colorimetric (λ=570 nm) or fluorometric (Ex/Em=535/587 nm) methods. Palmitic acid was used to generate a standard curve.

Free fatty acid assay (FFA) protocol:
Add edmedia sample from both groups (AUM with and without lipase) and standards to 96 well plate;
Mixed and incubated for 30 min at 37° C.; and
Quantified and analyzed FFA using a spectrophotometry microplate reader.

The results, illustrated in FIG. 6, show that after 7 days, there was no detectable degradation of microspheres in the AUM without lipase. This behaviour is what is expected to occur in the human bladder environment since healthy human urine does not contain lipase. This is an indication that the microspheres will last and hold their shape and form in the bladder for long periods of time.

### Particle Surface Erosion:

Scanning electron microscope images (FIG. 7) confirmed that there was no surface erosion occurring in the group of microspheres that was exposed to AUM, pH 7.5, without lipase for 7 days. That is, the images of the microspheres after 7 days are not noticeably different from the images taken after 1 hour.

### Example 8

### Carrier Evaluation:

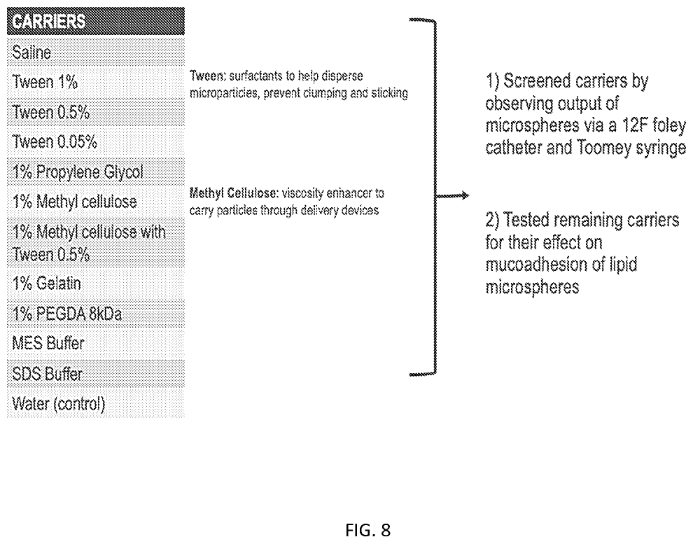

FIG. 8 outlines the list of carriers and vehicles that were tested to improve the deliverability of microspheres through a foley catheter. Briefly, 30 mL of each of the carrier solutions listed in FIG. 8 were mixed with 5 grams of microspheres and loaded into a Toomey syringe. The Toomey syringe with the drug product and carrier was then used to inject through a 12Fr foley catheter. The output of product through the foley catheter was rinsed with DI water, dried, and weighed to determine the amount that can be delivered and the product that is lost in the Toomey syringe and catheter.

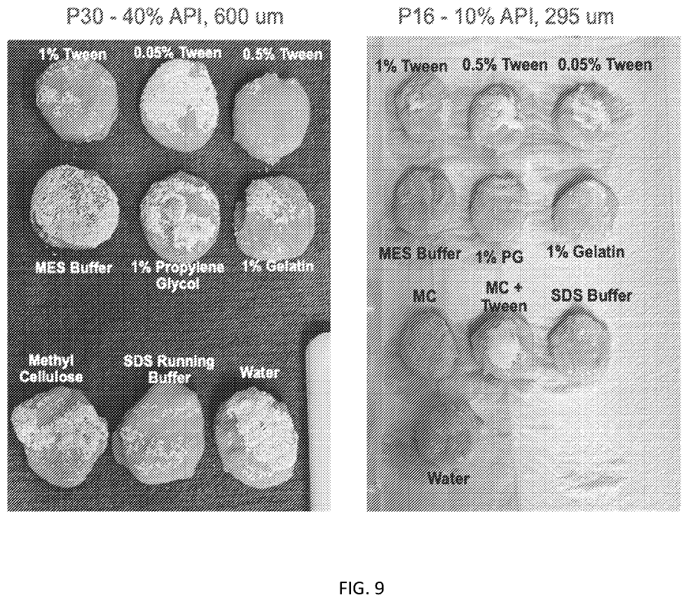

FIG. 9 provides the results of our investigation into whether any of the carriers or vehicles that the microspheres came into contact affected their mucoadhesion properties. To test this, the microspheres were immersed in the different carrier solutions of FIG. 8 and then placed onto the mucosal surface of a round tissue section from the bladder of a cow. The tissue sections, after placing the microspheres on it, were lifted and dipped in DI water. FIG. 9 provides the images obtained. With some carriers, such as 0.05% Tween (Tween 20 or Tween 80) and methyl cellulose, the microspheres continued to adhere to the tissue.

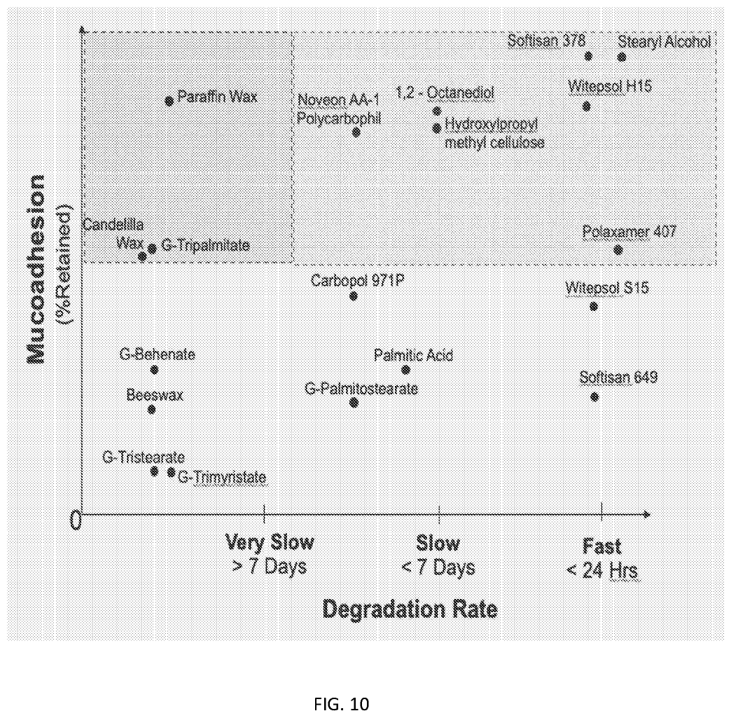

Ideal buoyant carriers are those that exhibit good mucoadhesion and a slow degradation rate, which in turn provides for long-term particle retention in the bladder and sustained drug release. FIG. 10 illustrates the results of particles prepared with the carriers indicated in the figure, with mucoadhesion shown as the percent retained over time and degradation rate shown over a period of less than 24 hours to over 7 days. Materials in the upper left quadrant are both mucoadhesive and degrade slowly, while materials in the upper right quadrant are excipients that degrade at a faster rate and can be utilized to speed up drug release. Placebo microspheres (not having any API) were developed using a combination of materials shown in FIG. 10.

Then, using the results, particles were prepared using the following as controlled release carriers:
1) Glyceryl Tristearate only (control)
2) 90% Glyceryl Tristearate: 10% Stearyl Alcohol
3) 90% Glyceryl Tristearate: 10% Hydroxypropyl methyl cellulose
4) 90% Paraffin wax: 10% Stearyl Alcohol
5) 90% Paraffin wax: 10% Hydroxypropyl methyl cellulose (HPMC)
6) 90% Paraffin wax: 10% Poloxamer 407
7) 90% Paraffin wax: 10% Softisan 378
8) 90% Paraffin wax: 10% Witespol S58
9) 90% Glyceryl Tripalmitate: 10% Stearyl Alcohol
10) 90% Glyceryl Tripalmitate: 10% NoveonAA (poly acrylic acid polymer)
11) 90% Glyceryl Tripalmitate: 10% Witepsol S58
12) 90% Glyceryl Tripalmitate: 10% HPMC
13) 90% Glyceryl Tripalmitate: 10% Poloxamer 407

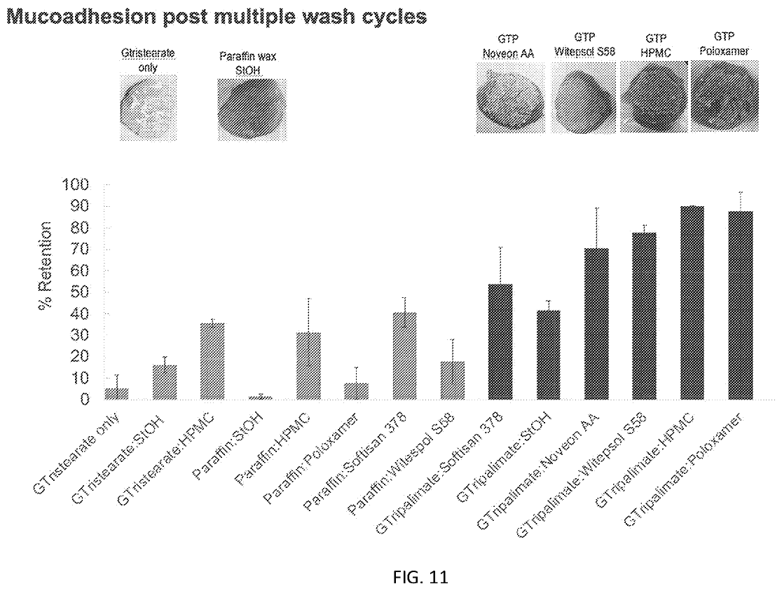

Particles prepared with the above controlled release carriers were tested for their mucoahesive ability by slathering 2 grams of microspheres on the mucosal surface of round tissue sections from cow bladder and then dipping the tissue sections in DI water, and measuring the microspheres that remained adherent on the tissue section. Percent retention of microspheres was calculated by the microspheres that remained adherent on the tissue section dividing by original amount that was slathered on the tissue section. The results of this experiment are shown in FIG. 11.

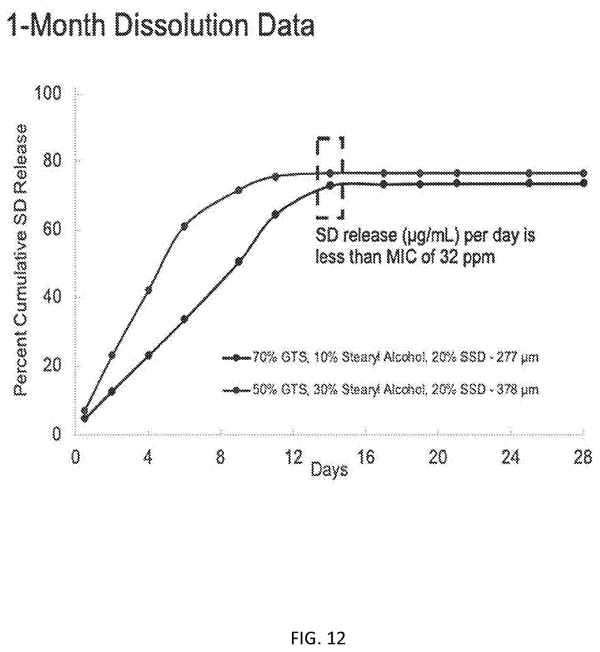

FIG. 12 demonstrates that drug release can be sped up by incorporating one of the materials set out in FIG. 10 In FIG. 12, drug (SSD) release from two batches of particles with different amounts of stearyl alcohol as carrier (10% and 30% by weight). Stearly alcohol (StOH) is an excipient that degrades faster than glyceryl tristearate alone. As shown in FIG. 12, formulations with higher amounts of StOH (30 wt. %) release SSD at a faster rate than formulations with 10 wt. % StOH. Drug release was evaluated in artificial urine media at pH 7.5 where microspheres were immersed in the media. 90 mL of diluent was removed at every sampling interval. Samples were evaluated using LC-MS for detection of SSD release over time.

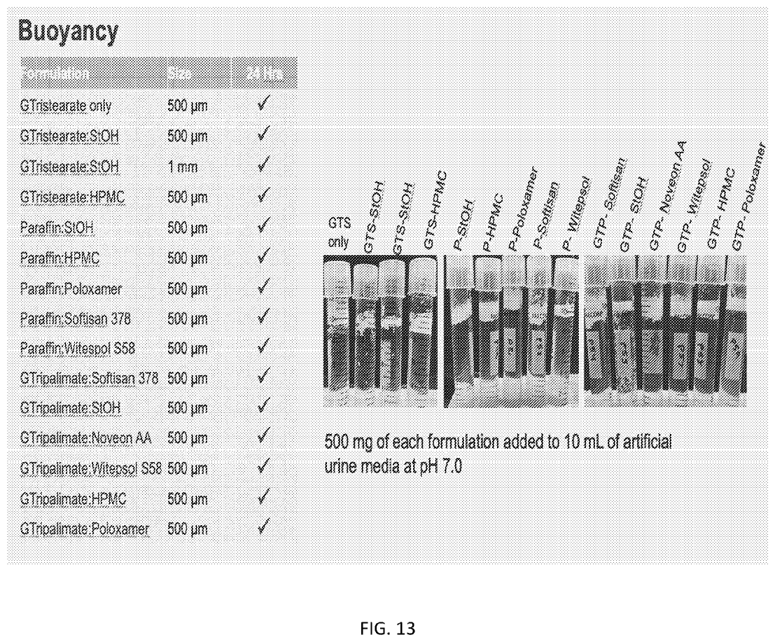

Then, particles formulated with the controlled release carriers enumerated above were evaluated for their ability to exhibit buoyancy in artificial urine media for 24 hours. 500 mg of each of the formulations above were placed in a tube with 10 ml of AUM. The tubes were then placed in an incubator at 37° C. and mixed at 100 rpm for 24 hours. Then, images were taken of each of the samples to evaluate whether particles formulated with the different combinations of controlled release carriers were buoyant after 24 hrs. The results are shown in FIG. 13.

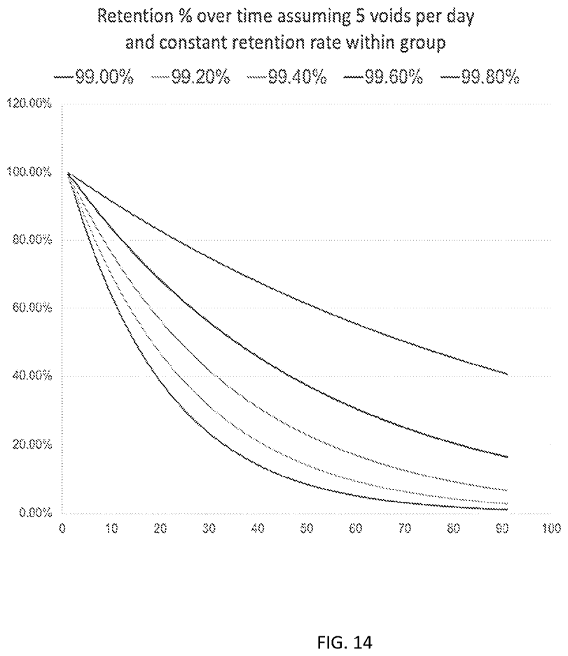

FIG. 14 models rate of retention in a single void. For example, assuming an individual voids 5 times per day and the rate of retention is 99%, then the amount of product remaining after one day is 1*0.99\hat{ }(5 voids×1 day)=95%. After 30 days, the amount of product left would be 1*0.99 (5 voids×30 days)=22%. This is charted for different retention rates—99%-99.8%.

### Example 9

### Effect of Shape on Drug Release

In this example various shapes of carrier particles were compared. The molecules were prepared by various methods including direct compression, solid lipid extrusion, and melting techniques. To formulate different shapes and geometries techniques such as injection molding, melt-spray congeal, compression, solid lipid extrusion via hot melt extrusion, and casting technique using 3D printed molds were used.

The tested particles included cylindrical rods with diameters ranging from 2-5 mm and heights of 5-15 mm that were formulated with melting polymer with drug and injecting in a silicone tube mold of desirable size. Spheres varying in diameter size from 100 μm-1 mm were formulated using melt spray congeal with atomizer. Spheres above 1 mm in diameter were formulated by casting hot melt mix of polymer and drug in 3D printed molds.

Product retention and tolerability in the bladder is dependent on buoyancy. To remain buoyant, drug product density must be within the specific gravity range of water or urine with density of water at 0.997 g/mL and density of urine at a range of 1.005-1.030 g/mL.

Buoyancy and retention in the bladder are affected by several factors:
1) Density—function of dosage form buoyancy that is dependent on the density of the surrounding media,
2) Size—Dosage form units with a diameter of more than 2 mm are reported to have 90%-95% retention in the bladder.
3) Shape of dosage form—single geometry (cylinder, sphere, hemisphere, ellipsoid, torus (ring shaped, cub, hexagonal prism, truncated cone, dodecahedron, etc. with flexural modulus), multiple unit (mixed geometries, same geometry of different sizes), expandable forms (change in shape or expand in size upon contact with surrounding environment triggered by pH, temperature, wettability like with swellable materials, or manual mechanical expansion as in inflating a balloon) are favorable towards retention in the bladder. Multiple unit formulations allow coadministration of units with different release profiles or containing incompatible substances and permit a larger margin of safety against dosage form failure compared with single unit dosage forms.
4) Coagulation of multiple forms—formulation of a single geometry or multiple different geometries adhered and coagulate in the presence of water/urine to form a single, bulk formulation of a size larger than the diameter of the urethra therefore enhancing its retention. This can occur if the formulation is coated by adhesive or swellable polymer chains that have affinity for each other when they are wet.

The formulation can be engineered to be buoyant by several methods:
1) Microporous compartmental systems—The drug is encapsulated inside a micro porous polymer matrix to form drug delivery vehicle or formulation. The peripheral walls of the formulation are completely sealed by dip coating in a polymer melt to prevent any direct contact with the surrounding media.
2) Hollow core or apertures in which the hollow core consists of drug reservoir protected by polymer matrix or the hollow core is filled with air surrounded by drug embedded in polymer matrix. Variations of this include more than one hollow cores at different locations, directions, and sizes. The core may be filled with vacuum, air, or inert gas.

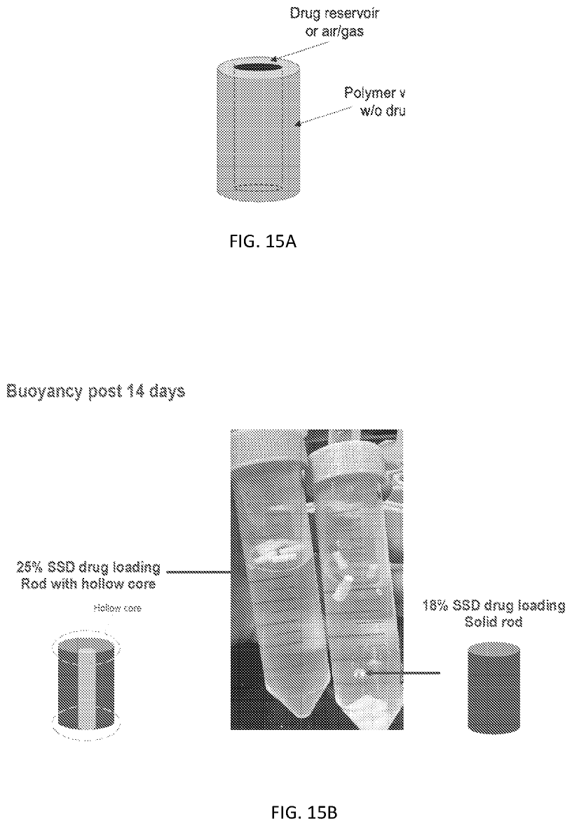

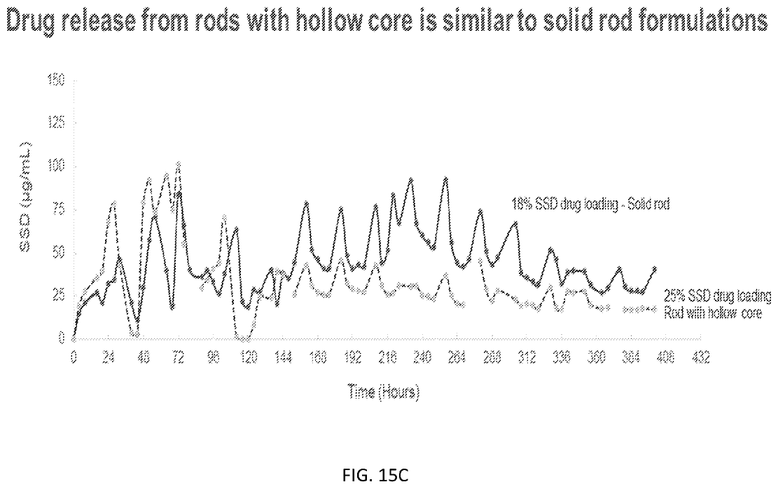

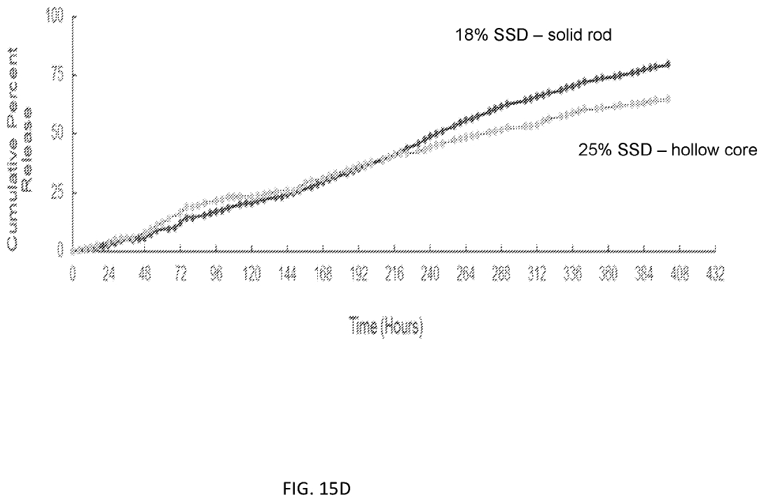

Four carrier particles were tested, as shown in FIG. 15 B:
Small sphere (2 mm)
Large sphere (4 mm)
Small cylinder (2 mm×4 mm)
Large cylinder (2 mm×10 mm)
   As described below, silver sulfadiazine (SSD) was used for further studies of cylindrical carrier particles.

### Solid Versus Hollow Core

The next study involved comparing the release of SSD over time in cylindrically shaped particles (solid and hollow). In brief, SSD was conjugated to the particles and incubated in solution. The concentration of SSD in the solution was measured at regular intervals from t₀ to 430 hours.

FIG. 15B shows the concentration of SSD over time. Rods were loaded with 18% SSD (top) or 25% SSD (hollow). For both types of particles, the concentration of SSD remained relatively constant for the entire duration of testing (i.e., to 420 hours). Similarly, FIG. 15C shows the concentration percent release over time. A straight line indicates that the active agent (i.e., SSD) is released steady.

### Example 10

### Amikacin Solubility Effects Release Kinetics

Several antimicrobial agents were considered for use as active agents with carrier particles.

TABLE 7

| | | | Max Drug | |
|---|---|---|---|---|
| | MIC₉₀ | Density | Loading | Indicated |
| Agent | (ppm) | (g/ml) | (%) | for UTI |
| Silver sulfadiazine* | 32 | 1.45 | 20 | X |
| Amikacin* | 4 | 1.6 | 18 | Yes |
| Tobramycin | 8 | 1.5 | 20 | Yes |
| Cefoperazone-sulbactam | 4 | 1.8 | 16 | Yes |
| Cefoperazone-avibactam* | 0.25 | 1.5 | 20 | Yes |
| Cefoperazone-tazobacatam* | 0.5 | 1.5 | 20 | Yes |
| Piperacillin-tazobactam | 4 | 1.5 | 20 | Yes |
| Chlorohexidine | 3 | 1.06 | 28 | X |
| Doripenem | ≤0.12 | — | — | Yes |
| Ertapenem* | 0.03 | 1.6 | 18 | Yes |
| Imipenem* | ≤0.12 | 1.6 | 18 | Yes |
| Meropenem | ≤0.06 | 1.4 | 21 | X |
| Cefepime | 8 | 1.6 | 18 | Yes |
| Cefoxitin | 16 | 1.6 | 18 | X |
| Ceftazidime | 8 | 1.6 | 18 | Yes |
| Tigecycline* | 0.25 | 1.5 | 20 | X |
| Colistin* | ≤0.5 | — | — | X |
| Polymxin B* | 1 | — | — | X |
| Aztreoman | 16 | 1.83 | 16 | Yes |

Tested against both MDR Ecoli and non-MDR Ecoli isolates (Others tested against non-MDR Ecoli isolates only)

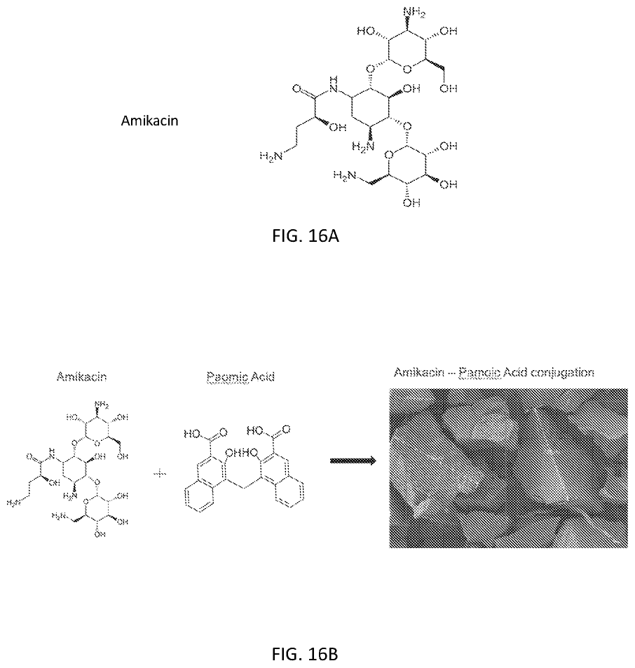

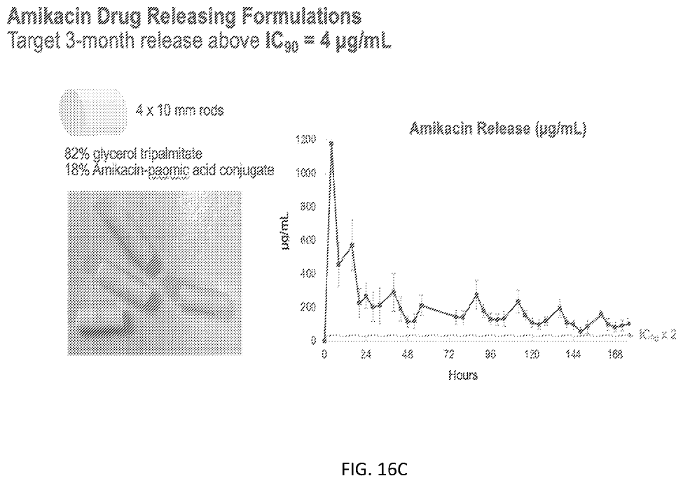

Amikacin is an aminoglycoside used to treat infections caused by more resistant strains of Gram-negative bacteria and some Gram-positive bacteria. It is highly soluble in water at 50 mg/ml. Formulations with free Amikacin caused 100% release in less than four hours. In this example, Amikacin was conjugated to Paomic Acid via the following steps.
1) A solution of Amikacin was prepared in water in the presence of acid;
2) A solution of pamoic acid was prepared in water in the presence of base;
3) The solutions were mix until solids precipitated;
4) The final step entailed filtering and drying the solid via lyophilization or vacuum oven drying.
   The structure of Amikacin is shown in FIG. 16A. Amikacin conjugated to Pamoic Acid (FIG. 16B) slowed its release by reducing reaction time in urine.

Next, the conjugate was attached to carrier particles (4×10 mm rods). The rate of release of Amikacin in solution (i.e., free Amikacin) was measured over time. The results are graphically depicted in FIG. 16B.

### Example 11

### Effect of Shape on Drug Release

In this example the release characteristics of different shapes were studied.

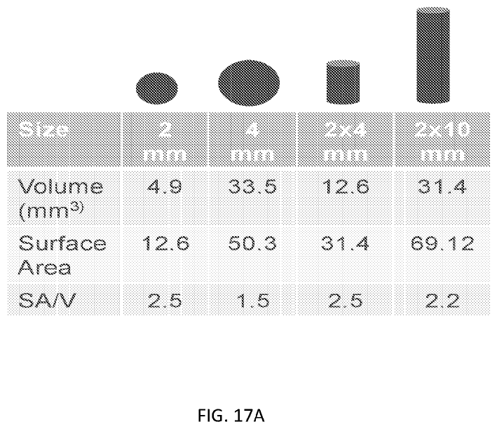

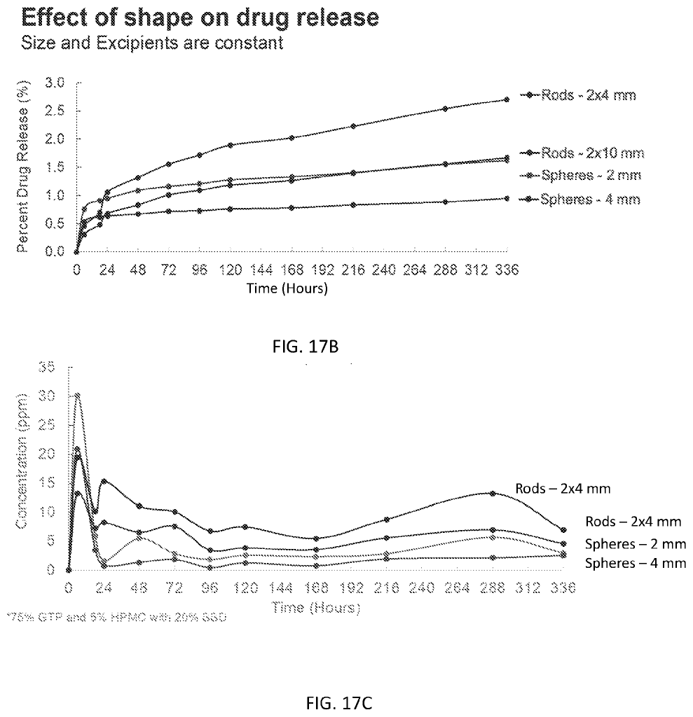

FIG. 17A shows the four shapes used in this example:
Small spheres (2 mm)
Large spheres (4 mm)
Small rods (2×4 mm)
Large rods (2×10 mm)

An ideal controlled-release particle maintains medication levels at a relatively constant (and pharmaceutically effective) level for a specific time period. FIG. 17B shows the percent of drug released over time for the four carriers tested. The large rods demonstrated the most preferable rate of drug release. The percent of drug released gradually increased to 336 hours. Small rods and small spheres had lower rates of drug release. The large spheres appeared least effective.

Similarly, FIG. 17C shows the concentration drug (ppm) over time for the four carriers tested. As above, the large rods demonstrated the most preferable rate of drug release. The concentration of drug (ppm) remained relatively constant from t₀ to 336 hours.

### Example 12

### Effect of Different Excipients on Drug Release

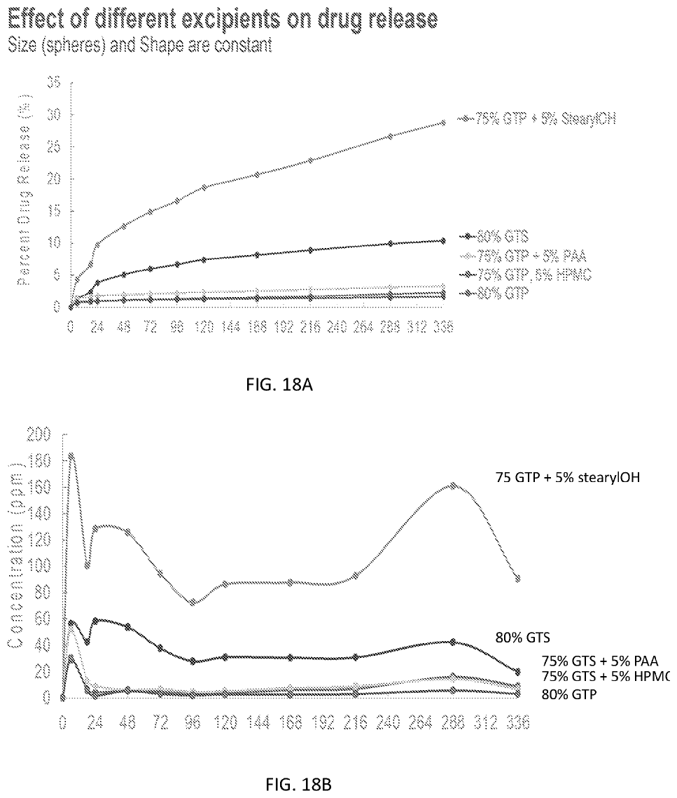

In this example, the effects of different excipients were compared. Materials that degrade faster and/or are more hydrophilic will exhibit faster drug release. The same shape (spheres) was used to compare five carrier particles:
75% GTP+5% stearyl alcohol
80% GTS
75% GTP, 5% HPMC
80% GTP
   FIG. 18A shows the percent of drug released over time for the four carrier particles tested. The first carrier (75% GTP+5% stearyl alcohol) degraded at the highest rate and demonstrated the most preferable rate of drug release.

Similarly, FIG. 18B shows the concentration drug (ppm) over time for the four carriers tested. As above, the first carrier (75% GTP+5% stearyl alcohol) demonstrated the most preferable rate of drug release. The concentration of drug (ppm) remained relatively constant from t₀ to 336 hours.

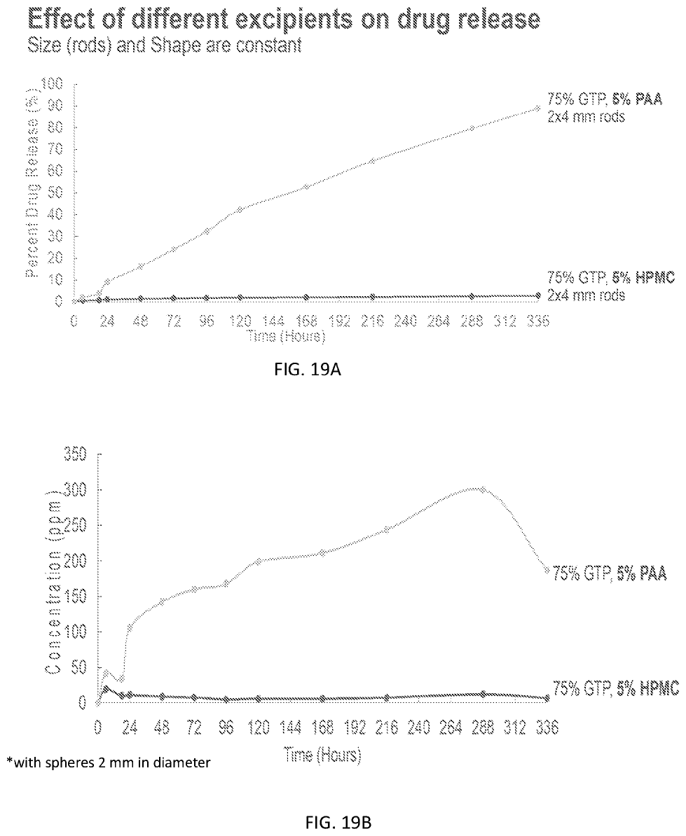

The study was repeated using rod-shaped particles (2×4 mm). The following carrier particles were studied:
75% GTP+5% PAA
75% GTP+5% HPMC
   The results are shown in FIG. 19A and FIG. 19B. The carrier with PAA degraded at the highest rate and demonstrated the most preferable rate of drug release. In contrast, the carrier with HPMC remained relatively stable with minimal drug release.

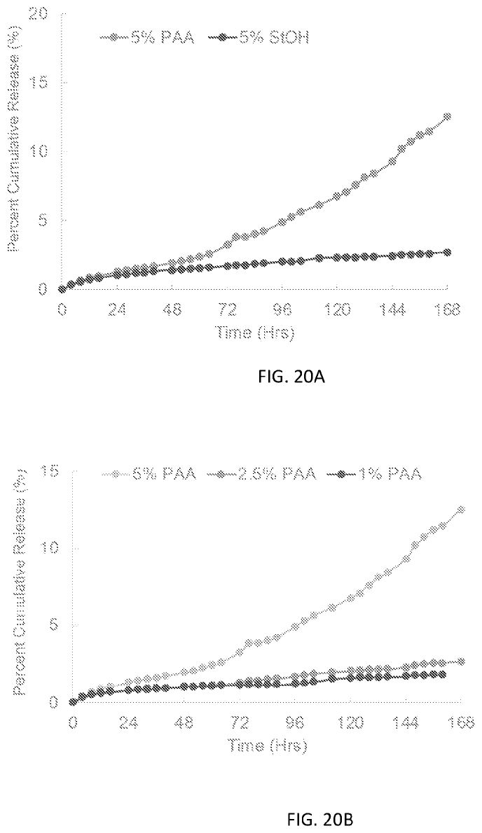

The following formulations were further studied:
75% GTP+5% PAA
75% GTP+5% StOH
   The results are shown in FIG. 20A. The carrier with PAA demonstrated the most preferable rate of drug release. In contrast, the carrier with StOH demonstrated minimal drug release.

Based on the above results, rods of various amounts of PAA were compared. Specifically, the following formulations were further studied:
75% GTP+5% PAA
75% GTP+2.5% PAA
75% GTP+1% PAA
   The results are shown in FIG. 20B. The carrier with 5% PAA demonstrated the most preferable rate of drug release. In contrast, the carriers with lower amounts of PAA demonstrated lower levels of drug release.

### Example 13

### Silver Sulfadiazine Drug Releasing Formulation—Varying Amount of PAA

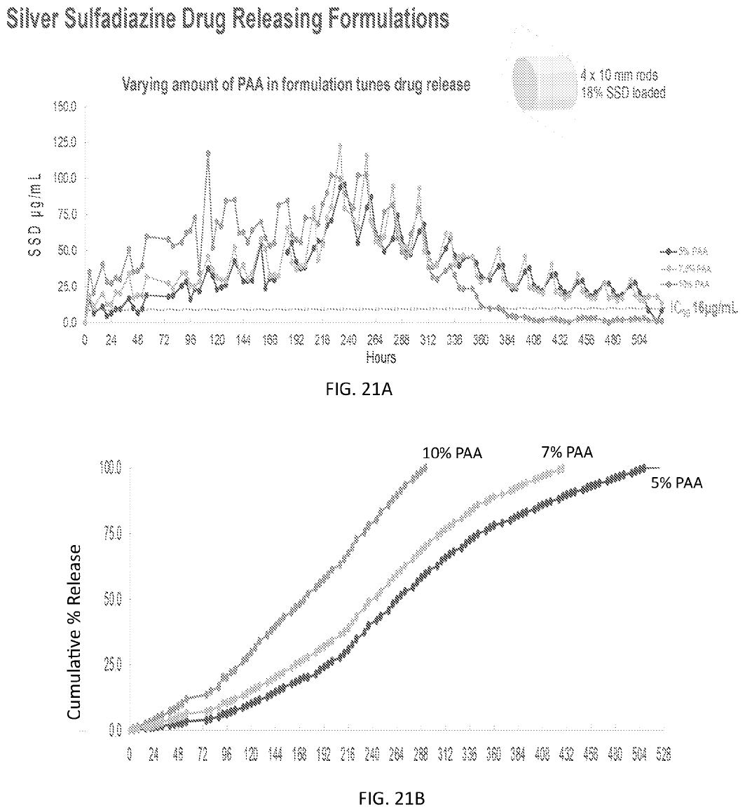

To identify a preferred concentration of PAA, rods with higher amounts of PAA were compared. Specifically, the following formulations were further studied on rods (4×10 mm) loaded with 18% SSD:
75% GTP+5% PAA
75% GTP+7.5% PAA
75% GTP+10% PAA
   The results are shown in FIG. 21A and FIG. 21B. The carriers with 5% PAA and 7.5% demonstrated similar concentrations of SSD. The higher PAA (10%) demonstrated the most rapid rate of drug release.

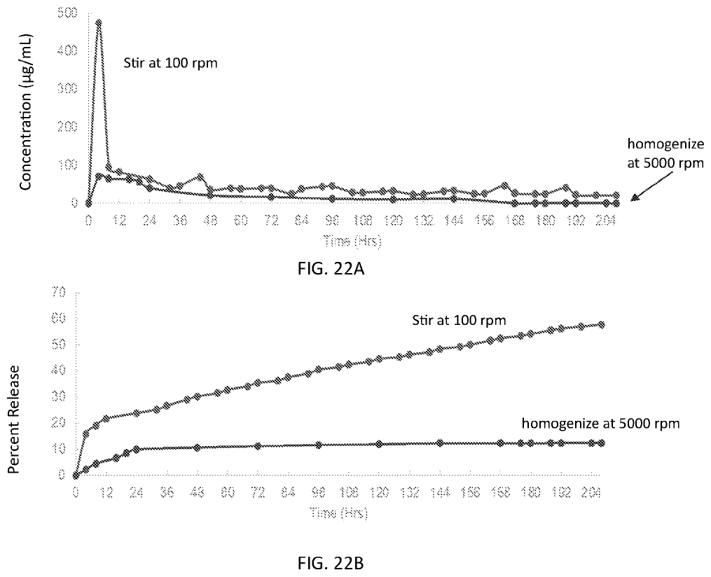

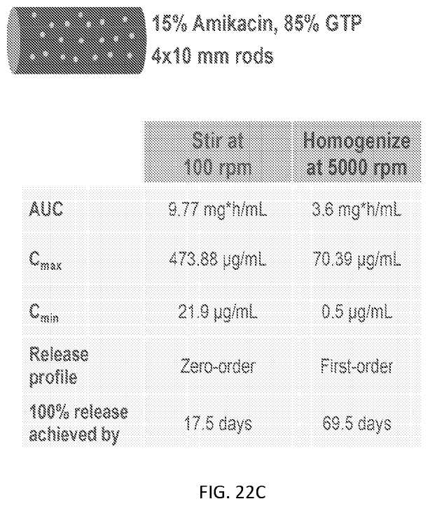

Based on the above results, rods prepared by different methods were studied. The rods (4×10 mm) contained 85% GTP and 15% Amikacin. The following preparations of rods were compared:
Stirred (Stirred at 100 rpm)
Homogenized (Homogenized at 5,000 rpm)
   The results are shown in FIGS. 22A and 22B. The stirred carrier demonstrated a rapid rate of drug release. In contrast, the homogenized carrier demonstrated a slower level of drug release.

Characteristics of each type of rod are listed in FIG. 22C. The stirred carrier demonstrated zero-order release profile with 100% released in 17.5 days. In contrast, the homogenized carrier demonstrated a first-order release profile with 100% released in 69.5 days.

### Example 14

### Outer Coating to Tune Drug Release

Dip or spray coating the final form of the formulation in solvents like acetone and ethanol, or polymer/excipients can slow the drug release by providing an extra barrier on the perimeter and exterior of the formulation for drug release to occur. The viscosity of the polymer melt, polymer/excipient mixture, duration of the dip, percent of solvent (v/v) in solution all affect the thickness of the coating and how tightly packed the barrier is to the final form (e.g., like a woven sweater).

Dip coating can happen in partial where some portion of the formulation is coated and the remaining is not or the entire rod/formulation could be dipped and therefore coated.

Materials with Tunable Degradation can be Utilized to Control Drug Release

Materials in the bladder that have been tested by the inventors for drug delivery in the bladder include the following classes proprietarily known as Dynasan®, Softisan®, Witepsol®. These materials differ by their melting point and hydroxyl value. Materials with lower melting point closer to body temperature (e.g., 30-39° C.) will degrade faster than materials with higher melting points. The hydroxyl value is defined as the number of milligrams of potassium hydroxide (KOH) required to neutralize the acetic acid taken up on acetylation of one gram of a chemical substance that contains free hydroxyl groups. Hydroxyl is a common hydrophilic group and can be used to modify the surface and change the wettability of the excipient which is owed its ability to form hydrogen bonds with surrounding water. The density of the hydrogen bond formed between hydroxyls and water molecules in the adsorption layer contributes to the overall wettability of the excipient. Increase in wettability can increase degradation of excipients via hydrolysis and thereby enable drug release. Both properties of materials are taken into account to select the base, predominant excipient for formulation or combination of several excipients to achieve the ideal degradation and drug release profile and rate. Base polymer material can include a hydrophobic material with high melting point such as glyceryl tripalmitate (also known as Dynasan® 116) or glyceryl tristearate (e.g., Dynasan® 118) or glyceryl monostearate (e.g., Dynasan® 114) at range of 50%-95% (w/w) mixed with an excipient at relative range of 1%-50% (w/w) to enhance degradation and drug release. The polymer mixed can be melted and then mixed with drug (or API) at range of 1%-25% (w/v).

Several fast-degrading materials were tested as rapid releasing formulations as summarized in Table 8 below.

TABLE 8

| | Witepsol ®E 76 | Witepsol ®H 15 | Witepsol ®H 19 | Witepsol ®S 55 | Witepsol ®S58 |
|---|---|---|---|---|---|
| Melting point | 37-39 | 31-38 | 30-39 | 30-39 | 32-32 |
| Hydroxyl value | 30-40 | 5-15 | 20-30 | 50-65 | 60-70 |
| Buoyancy after | Not byouant | byouant | byouant | byouant | byouant |
| 24 hours | | | | | |
| Degradation | Not fully | 100% degraded | 100% degraded | degraded into | 100% degraded |
| | degraded | at 6 h | at 6 h | small pieces | at 1 h |
| Notes | Did not | Forms a soft | Small particles | Small fragments | Fully degraded - |
| | degrade- | wax at room | form at room | ovserved at | no fragments |
| | formed a blob | temp | temp | room temp | observed |

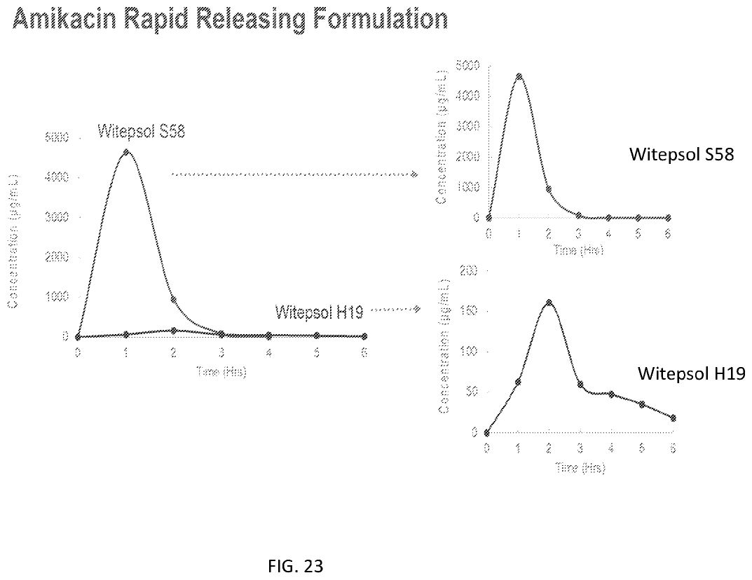

Based on the above results, rods prepared by using Witepsol S58 and H19 as rapid releasing formulations. The results are shown in FIG. 23. The particles with Witepsol S58 released amikacin rapidly (i.e., peak concentration of 5000 μg/ml was reached in about one hour). In contrast, the particles with Witepsol H19 released amikacin slowly (peak concentration of 150 μg/ml was reached at about two hours).

### Example 15

### Adjusting Crystallinity

Polymorphs occur when a polymer solidifies in different chemically identical lattice structures that therefore have different tertiary and quaternary structure. Adjusting crystallinity of product or manufacturing different polymorphs enables tuning drug release, buoyancy, density, material properties such as hardness, stability, packing, and appearance. In this example alpha (α) and beta (β) crystalline structures were produced and compared.
Alpha (α): based on theoretical polymer lattice, this would look “glossy” because the lattice is highly ordered, and carbon chains are laid parallel so there is a smooth surface in the quaternary structure. To create alpha, the system's energy must be lowered to reduce the movement along the monomers degrees of freedom and lower the intramolecular hindrance.
Beta (β): Would look more randomly packed and “chalky” because the monomers have bent glycerol carbon chains. If beta is more randomly packed, then energy must be stored between the polymer units during the polymerization process.

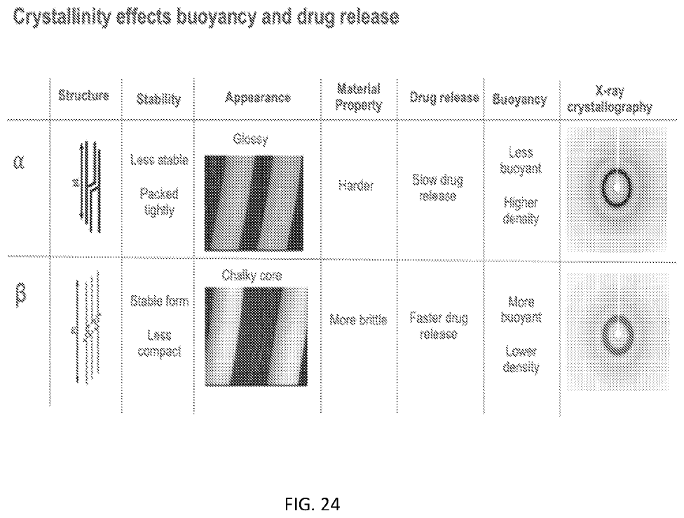

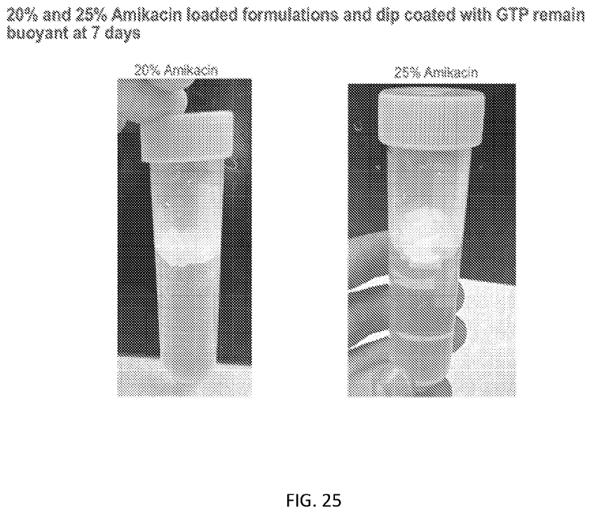

Characteristics of each type of crystalline structure are compared in FIG. 24. Factors that can be tuned to achieve beta or alpha structure include:
Rate of cooling and temperatures: Cooling down slowly at room temp (e.g., 25° C.) creates β crystalline structure. Rapidly cooled at 4° C. creates α crystalline structure.
Curing rate: Rods cured at 115° F./55° C. in the oven for 14 hours creates β crystalline structure.
Number of melting cycles: Melting the polymer, resolidify it, and melting again creates β crystalline structure.
Surface area and size of the rods: Larger rods take longer to cool down and the probability of β crystalline structure forming with 5×15 mm rods is more likely than with 4×5 mm rods.
Effects of the drug or excipients on polymorphism: The addition of polar drug or excipient would favor β crystalline structure. Different carbon end groups such tristearate vs tripalmitate effect crystallinity.

### Example 16

### Coatings

A prolonged circulation is a requisite for effective drug delivery and therapeutic efficacy. Coatings around the rod of different polymers and excipients can enhance buoyancy of the rods by covering pockets of air/pores. Without the coatings, pores or air pockets are replaced by the surrounding media/urine and thereby cause the rods to sink. In aspects, a coating helps exclude water, keeps particle buoyant and/or extends/control drug delivery rate. In aspects, a coating alters the rate of particle degradation.

Various factors can be fine-tuned to achieve ideal coating thickness-exposure time, curing temperature, solvent type, solvent concentration, solvent-polymer (v/v) ratio, synthetic and natural polymers (i.e. Poly(ethylene Glycol) PEG, glycerol tripalmitate, glycerol tristearate, poly(lactic-co-glycolic acid), Poly(caprolactone), and etc.) such that its addition to the rod as a coating does not increase the overall density above the density of urine.

The above discovery also enables increased drug loading while not compromising buoyancy. Coatings can be manufactured through various processes-dip coating rods directly into hot melt of polymer and solvent mixture or spray coating. Coatings can slow the drug release and burst release by acting as a barrier for the drug to diffuse out. This can be adjusted by thickness of the coating or by coating sections of the rod (half of the rod vs full rod) or different polymers/excipients that vary in hydrophobicity.

For example, increasing drug loading from 15% to 20% and 25% did not compromise buoyancy and thereby allows for increase drug loading per rod. See FIG. 25.

### Example 17

### Treatment of Chronic UTI

In this example, an elderly female patient visits a healthcare provider with signs/symptoms of a UTI. The provider notes that the patient has had three UTI's within a six-month period. Previously, she has been prescribed oral antibiotics (i.e., amoxicillin or nitrofurantoin). Although prophylactic antibiotics are an option, the patient wishes to avoid them. Further, the healthcare provider acknowledges the risk of antibiotic resistance.

The provider presents the option of using intravesicular controlled release particles. Conventional drug delivery systems (e.g., tablets, capsules, etc.) can suffer from poor bioavailability and fluctuations in plasma drug levels. The use of intravesicular particles allows drug delivery at a specified controlled rate and at a target site for greater efficacy and safety.

Particles were prepared as described herein. Specifically, rod-shaped particles contained 85% GTP and 15% Amikacin. Relatively large particles are used (i.e., rods of 2.0 to 2.5 mm in length). The provider administers the particles using a 5 mm syringe (i.e., transurethral). Thereafter, the presence/location is observed via ultrasound.

The provider notes that most of the particles remain in the bladder because (a) they tend to stay away from the urethral opening during the first half of the voiding cycle when the urethral opening is at its largest; (b) as the bladder empties and the urethral opening gradually narrows, the particle diameter is wider than the narrowed urethral opening in the second half of the voiding cycle and as voiding approaches completion; (c) at the end of the voiding cycle when the urethral opening is narrowed and the buoyant particles are brought to the level of the urethral opening, the particles tend to aggregate within the bladder and prevent outflow of the particulate formulation before substantially complete release of the active agent.

The particles deliver medication to specifically increases the drug concentration in the bladder/urethra. Benefits include:
No requirement to regularly consume antibiotics (tablets, capsules, etc.)
Low doses of antibiotic are effective (lower amounts are necessary for local rather than systemic use)
Lower likelihood of adverse/unwanted side effects
Low likelihood of antibiotic resistance
Avoids first-pass metabolism
Improved therapeutic window

The patient reports alleviation of symptoms within six hours of the procedure. The patient has no discomfort nor any feeling of the presence of the particles. The patient is evaluated one week later and has no signs/symptoms of infection. Further, the provider notes that there is no detectable circulating amikacin though it is present in the bladder at an effective concentration. An ultrasound is performed and about 90% of the particles remain present in the bladder. A second ultrasound is performed four weeks later and about 10% of the particles are present. Thereafter, the patient is advised to maintain routine visits to her healthcare provider and to report any signs/symptoms of possible UTI.

### Example 18

### Treatment of Over-Active Bladder

Overactive bladder can result when muscles of the bladder start to contract on their own even when the volume of urine in your bladder is low. These “involuntary contractions” create an urgent need to urinate. Several conditions may contribute to signs and symptoms of overactive bladder, including: neurological disorders (e.g., stroke and multiple sclerosis), diabetes, UTIs that can cause symptoms similar to those of an overactive bladder, hormonal changes during menopause, tumors, bladder stones, enlarged prostate, constipation or previous surgery to treat incontinence.

In this example, an elderly male patient visits a healthcare provider with signs/symptoms of overactive bladder. He complains of a frequent and sudden urge to urinate that is difficult to control. He describes the feeling of a need to pass urine many times during the day and night. He also has frequence unintentional loss of urine (i.e., urgency incontinence). Behavioral therapies (i.e., pelvic floor muscle exercises, exercise and dietary changes) have not been effective.

Oxybutynin is an antispasmodic that can help decrease muscle spasms of the bladder and the frequent urge to urinate caused by these spasms. Oxybutynin is indicated in patients with overactive bladder or symptoms of detrusor overactivity, including urinary frequency and urgency. The provider presents the option of using intravesicular controlled release particles with oxybutynin to help relax the muscles of the bladder. Particles were prepared as described herein. Specifically, rod-shaped particles rods (4×10 mm) contained 85% GTP and 15% Oxybutynin. The particles are administered to the patient using a 5 mm syringe.

The patient reports alleviation of symptoms within two hours of the procedure. The patient has no discomfort nor any feeling of the presence of the particles. The patient is evaluated one week later and reports 80-90% reduction in signs/symptoms of over-active bladder. Further, the provider notes that there is no detectable circulating (i.e., systemic) oxybutynin. The patient is advised to maintain routine visits to his healthcare provider and to report any symptoms of over-active bladder. If the symptoms return, the healthcare provider will discuss the possibility of repeating the treatment.

### Example 19

### Treatment of Interstitial Cystitis

Interstitial cystitis is a chronic condition causing bladder pressure, bladder pain and sometimes pelvic pain. The pain ranges from mild discomfort to severe pain. The condition is a part of a spectrum of diseases known as painful bladder syndrome. Interstitial cystitis most often affects women and can have a long-lasting impact on quality of life.

In this example, a 65-year-old female patient visits a healthcare provider with signs/symptoms of interstitial cystitis. Previously, she has used antihistamines along with nonsteroidal anti-inflammatory drugs with minimal or no improvement of symptoms.

Pentosan polysulfate sodium (Elmiron®) is a weak blood thinner used to treat discomfort or bladder pain associated with interstitial cystitis. The most common side effects of Elmiron are hair loss, diarrhea and nausea.

The provider presents the option of using intravesicular controlled release particles with pentosan polysulfate sodium. Conventional drug delivery systems (e.g., tablets, capsules, etc.) can suffer from poor bioavailability and fluctuations in plasma drug along with side-effects.

Particles were prepared as described herein. Specifically, rod-shaped particles rods (4×10 mm) contained 85% GTP and 15% pentosan polysulfate sodium. The particles are administered to the patient using a 5 mm syringe.

The patient reports alleviation of symptoms within 24 hours of the procedure. The patient has no discomfort nor any feeling of the presence of the particles. The patient is evaluated one week later and reports approximately 80% reduction in signs/symptoms of interstitial cystitis. The patient is advised to maintain routine visits to his healthcare provider and to report any discomfort. If the symptoms return, the healthcare provider will discuss the possibility of periodically repeating the treatment.

### Example 20

### Treatment of Bladder Cancer

Types of bladder cancer include urothelial carcinoma, squamous cell carcinoma and adenocarcinoma. In this example, a 75-year-old male patient visits a healthcare provider complaining of frequent and painful urination. He also reports the presence of blood in his urine. The healthcare provider assesses the patient and ultimately sends a tissue biopsy for diagnosis.

The patient is diagnosed with urothelial carcinoma. This type of cancer occurs in the cells that line the inside of the bladder. Urothelial cells expand when the bladder is full and contract when the bladder is empty. These same cells line the inside of the ureters and the urethra, and cancers can form in those places as well. Urothelial carcinoma is the most common type of bladder cancer in the United States.

A specialist recommends surgery followed by chemotherapy. Chemotherapy after surgery (i.e., adjuvant chemotherapy) can kill remaining cancer cells and reduce the chances that these cancer cells form new tumors. For the adjuvant chemotherapy, the specialist recommends intravesical chemotherapy to target cancers that are confined to the lining of the bladder but have a high risk of recurrence or progression to a higher stage. The patient agrees to the recommended course of treatment. The adjuvant therapy will be intravesical chemotherapy using gemcitabine.

The patient undergoes surgery and is allowed time to recover. Thereafter, the patient receives the adjuvant therapy using particles as described herein. Specifically, rod-shaped particles rods (4×10 mm) contain 85% GTP and 15% gemcitabine (Gemzar®). The particles are administered through the urethra directly into the bladder of the patient using a 5 mm syringe.

An ultrasound is performed one week after the procedure and about 90% of the particles remain present in the bladder. The approach allows an effective level of the agent to remain in the bladder for an extended period of time (i.e., greater than one month. Thereafter, the patient is advised to maintain routine visits to her healthcare provider and to report any discomfort.

Certain embodiments of the present invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the present invention to be practiced otherwise than specifically described herein. Accordingly, this invention includes all modifications and equivalents of the subject matter recited in the claims appended hereto as permitted by applicable law. Moreover, any combination of the above-described embodiments in all possible variations thereof is encompassed by the invention unless otherwise indicated herein or otherwise clearly contradicted by context.

In one embodiment, then, a controlled release pharmaceutical formulation is provided for intravesicular administration to a subject, the formulation can include a population of particles having a mean diameter greater than 2.0 mm and can include about 2.5 wt. % to about 95 wt. % of a pharmacologically active agent and about 5 wt. % to about 97.5 wt. % of a controlled release carrier effective to render the particles buoyant in urine and provide for sustained release of the active agent in the bladder throughout an extended drug delivery period generally in the range of about one month to at least about three months, e.g., about one month to about four months. According to some embodiments, the drug delivery period is in the range of about two to about four months, such as about three months. In some embodiments, the mean diameter of the particles is in the range of 2.0 mm to about 12.0 mm, 2.0 mm to about 6.5 mm, 2.0 mm to about 6.0 mm, about 2.5 mm to about 5.5 mm, about 2.5 mm to about 5.0 mm, about 2.5 mm to about 4.5 mm, about 2.5 mm to about 3.5 mm, or 2.0 mm to about 3.0 mm.

In aspects, the controlled release particles described herein are cylindrically-shaped (i.e., barrel-shaped or cylindrical) or substantially cylindrically-shaped. In aspects, they have a volume of about 10 to 50 mm³. In aspects, the particles have a volume of about 1 mm³, about 2 mm³, about 3 mm³, about 5 mm³, about 7 mm³, about 10 mm³, about 15 mm³, about 20 mm³, about 25 mm³, about 30 mm³, about 35 mm³, about 40 mm³, about 45 mm³, about 50 mm³, about 60 mm³, about 70 mm³, about 80 mm³, about 90 mm³, about 100 mm³ or larger. In aspects, the particles have a hollow core.

In aspects, the particles have a surface area of about 2 mm², about 3 mm², about 5 mm², about 7 mm², about 10 mm², about 15 mm², about 20 mm², about 25 mm², about 30 mm², about 35 mm², about 40 mm², about 45 mm², about 50 mm², about 60 mm², about 70 mm², about 80 mm², about 90 mm², about 100 mm² or larger.

In aspects, the controlled release particles described herein are spherically-shaped (i.e., round) or substantially spherically-shaped. In aspects, they have a volume of about 10 to 50 mm³. In aspects, the particles have a volume of about 1 mm³, about 2 mm³, about 3 mm³, about 5 mm³, about 7 mm³, about 10 mm³, about 15 mm³, about 20 mm³, about 25 mm³, about 30 mm³, about 35 mm³, about 40 mm³, about 45 mm³, about 50 mm³, about 60 mm³, about 70 mm³, about 80 mm³, about 90 mm³, about 100 mm³ or larger. In aspects, the particles have a hollow core.

In aspects, the particles have a surface area of about 2 mm², about 3 mm², about 5 mm², about 7 mm², about 10 mm², about 15 mm², about 20 mm², about 25 mm², about 30 mm², about 35 mm², about 40 mm², about 45 mm², about 50 mm², about 60 mm², about 70 mm², about 80 mm², about 90 mm², about 100 mm² or larger.

In aspects, the particles have a ratio of surface area to volume (SA/V) of about 1.2, about 1.5, about 1.6, about 1.7, about 1.8, about 1.9, about 2.0, about 2.1, about 2.2, about 2.4, about 2.6, about 2.8, about 3.0 or higher.

In aspects, an intravesical drug delivery formulation disclosed herein is capable of reducing the signs/symptoms associated with an ailment (e.g., urinary tract disorder) in an individual suffering from the ailment by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment.

In other aspects, an intravesical drug delivery formulation is capable of reducing the number of bacteria in the bladder/urethra in an individual suffering from an infection therein by, e.g., about 10% to about 100%, about 20% to about 100%, about 30% to about 100%, about 40% to about 100%, about 50% to about 100%, about 60% to about 100%, about 70% to about 100%, about 80% to about 100%, about 90% to about 100%, about 10% to about 90%, about 20% to about 90%, about 30% to about 90%, about 40% to about 90%, about 50% to about 90%, about 60% to about 90%, about 70% to about 90%, about 80% to about 90%, about 10% to about 80%, about 20% to about 80%, about 30% to about 80%, about 40% to about 80%, about 50% to about 80%, or about 60% to about 80%, about 70% to about 80%, about 10% to about 70%, about 20% to about 70%, about 30% to about 70%, about 40% to about 70%, about 50% to about 70% or about 60% to about 70% as compared to a patient not receiving the same treatment.

In other aspects, an intravesical drug delivery formulation disclosed herein is capable of reducing the recovery time associated with an ailment (e.g., urinary tract disorder) in an individual suffering from the ailment by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to a patient not receiving the same treatment.

In other aspects, an intravesical drug delivery formulation disclosed herein is capable of reducing the effective amount of a pharmaceutically active agent needed to treat an ailment (e.g., urinary tract disorder) in an individual suffering from the ailment by, e.g., at least 10%, at least 15%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90% or at least 95% as compared to conventional oral administration/systemic use of the active agent.

In aspects, the particles of the intravesical drug delivery formulation have a density of less than 1.030 g/ml. In aspects, they have a density of about 0.9 g/ml, about 1.000 g/ml, about 1.010 g/ml, about 1.020 g/ml, about 1.030 g/ml, about 1.040 g/ml, about 1.050 g/ml, about 1.060 g/ml, about 1.070 g/ml or greater.

All patents, patent publications, and other publications referenced and identified in the present specification are individually and expressly incorporated herein by reference in their entirety for the purpose of describing and disclosing, for example, the compositions and methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

1. An intravesical drug delivery system, the system comprised of a plurality of particles, each particle having an excipient portion and an active agent,
wherein the particles are buoyant in urine and the active agent is released upon exposure to liquid at a substantially constant release rate.

2. The intravesical drug delivery system of claim 1, wherein the particles are substantially cylindrically shaped or substantially spherically shaped.

3.-5. (canceled)

6. The intravesical drug delivery system of claim 1, wherein the excipient portion is comprised of a degradable material.

7. The intravesical drug delivery system of claim 1, wherein the active agent is a drug for the treatment of a disorder of the urinary system.

8. The intravesical drug delivery system of claim 1, wherein the active agent is one or more of an anti-infective agent, an anesthetic agent, an analgesic agent, a diuretic, an anti-inflammatory agent, a coagulant or an anti-coagulant, a chemotherapeutic agent, an agent for the treatment of incontinence, a renin-angiotensin-aldosterone system (RAAS) inhibitor, an agent for treating kidney stones or a contrast agent for diagnostics and monitoring.

9. The intravesical drug delivery system of claim 8, wherein the anti-infective agent is an antibacterial agent, an anti-fungal agent or an antiviral agent.

10.-13. (canceled)

14. The intravesical drug delivery system of claim 1, wherein the particles have a mean diameter of about 2.0 mm to about 8.0 mm, about 2.0 mm to about 6.0 mm or about 2.0 mm to about 4.5 mm.

15.-16. (canceled)

17. The intravesical drug delivery system of claim 1, wherein the particles are comprised of about 2.5 wt. % to about 25 wt. % of the active agent and about 5 wt. % to about 97.5 wt. % of the excipient.

18.-27. (canceled)

28. The intravesical drug delivery system of claim 1, wherein the particles are comprised of a matrix and the pharmacologically active agent is dispersed therein.

29. The intravesical drug delivery system of claim 1, wherein the particles are comprised of a coating on a core that comprises the pharmacologically active agent dispersed therein.

30.-45. (canceled)

46. A controlled release pharmaceutical formulation for intravesicular administration to a subject to treat a bacterial infection of the bladder or urinary tract, the formulation comprising:
a population of particles having a mean diameter in the range of 2.5 mm to about 4.0 mm and a mean specific gravity of less than 1.005, wherein the particles comprise:
50 wt. % to 95 wt. % of a controlled release carrier effective to render the particles buoyant in urine and provide for sustained release of the active agent in the bladder throughout a drug delivery period of at least about one month, wherein the controlled release carrier comprises a mucoadhesive material to facilitate particle retention by the bladder wall; and
5 wt. % to 50 wt. % of silver sulfadiazine dispersed within the carrier.

47. (canceled)

48. A method of treating an ailment in a subject, the method comprising intraurethral administration of the plurality of particles of claim 1 to a subject, wherein the ailment is a disorder of the urinary system.

49. A method of treating and/or preventing an ailment in a subject, the method comprising:
a) providing a plurality of particles, each particle having a buoyancy resulting in flotation of the plurality of particles in urine contained in a urinary bladder,
b) delivering the plurality of particles into the urinary bladder of the subject,
c) allowing excretion and/or degradation of the plurality of particles over a period of time,
wherein each of the plurality of particles is comprised of an excipient portion and an active agent, and wherein the active agent is released into the urinary bladder.

50. The method of claim 49, wherein the ailment is a urinary tract infection, bladder cancer, kidney cancer, ureter cancer, urethra cancer, anticoagulant disease, overactive bladder, underactive bladder, retained urine, diabetes, heart failure, kidney failure or cystitis.

51. The method of claim 49, where the active agent is one or more of a urease inhibitor, a chelating agent, an antibacterial agent and an enzyme.

52. The method of claim 49, wherein the excipient portion is comprised of a degradable material.

53. The method of claim of claim 49, wherein the active agent is a drug for the treatment of a disorder of the urinary system.

54. The method of claim 49, wherein the active agent is one or more of an anti-infective agent, an anesthetic agent, an analgesic agent, a diuretic, an anti-inflammatory agent, a coagulant or an anti-coagulant, a chemotherapeutic agent, an agent for the treatment of incontinence, a renin-angiotensin-aldosterone system (RAAS) inhibitor, an agent for treating kidney stones or a contrast agent for diagnostics and monitoring.

55. The method of claim 49, wherein the step of delivering the plurality of particles into the urinary bladder of the subject comprises intraurethral delivery.

56. The method of claim 49, wherein the period of time is more than one month.

57. The method of claim 49, wherein the particles are comprised of a matrix and the pharmacologically active agent is dispersed therein.

58. The method of claim 49, wherein the particles are comprised of a coating on a core that comprises the pharmacologically active agent dispersed therein.

59. The method of claim 49, wherein the particles are dispersed in a liquid vehicle for delivery into the urinary bladder of the subject,
wherein the liquid vehicle comprises at least one of a viscosity adjusting agent, a tonicity adjusting agent, a buffer and a dispersant

60. The method of claim 49, wherein the active agent is released into the urine over a period of about one month.

61. The method of claim 49, wherein the active agent is released into the urine at a substantially steady state.
